SEQUENCE LISTING (25 sequences)

SEQ ID NO: 1 - protein, 12 aa, synthetic construct
  source 1..12: note = PeptideX2
KSIAYLQMNSLK

SEQ ID NO: 2 - protein, 14 aa, synthetic construct
  source 1..14: note = G2PeptideX2
GKSIAYLQMNSLKG

SEQ ID NO: 3 - protein, 13 aa, synthetic construct
  source 1..13: note = Peptide X2-13
GKAAYLQMNAAKG

SEQ ID NO: 4 - protein, 13 aa, synthetic construct
  VARIANT 2: note = X = K or R
  VARIANT 12: note = X = K or R
GXAAYLQMNAAXG

SEQ ID NO: 5 - protein, 5 aa, synthetic construct
  source 1..5: note = pentide or peptideX2 core
YLQMN

SEQ ID NO: 6 - protein, 14 aa, synthetic construct
  source 1..14: note = spacer sequence
EGKSSGSGSESKVD

SEQ ID NO: 7 - protein, 18 aa, synthetic construct
  source 1..18: note = spacer sequence
KESGSVSSEQLAQFRSLD

SEQ ID NO: 8 - protein, 5 aa, synthetic construct
  source 1..5: note = spacer sequence
GGGGS

SEQ ID NO: 9 - protein, 8 aa, synthetic construct
  source 1..8: note = FLAG peptide epitope tag sequence
DYKDDDDK

SEQ ID NO: 10 - protein, 13 aa, synthetic construct
  source 1..13: note = K2R/K12R-substituted PeptideX2-13
GRAAYLQMNAARG

SEQ ID NO: 11 - protein, 13 aa, synthetic construct
  VARIANT 1: note = X = glycine (G) or proline (P)
  VARIANT 2: note = X = lysine (K) or arginine (R)
  VARIANT 3: note = X = alanine (A) or nothing
  VARIANT 4: note = X = alanine (A) or nothing
  VARIANT 10: note = X = alanine (A) or nothing
  VARIANT 11: note = X = alanine (A) or nothing
  VARIANT 12: note = X = lysine (K) or arginine (R)
  VARIANT 13: note = X = glycine (G) or proline (P)
XXXXYLQMNXXXX

SEQ ID NO: 12 - protein, 13 aa, synthetic construct
PKAAYLQMNAAKP

SEQ ID NO: 13 - protein, 11 aa, synthetic construct
GKAYLQMNAKG

SEQ ID NO: 14 - protein, 13 aa, synthetic construct
GKAAYLQMNAAKP

SEQ ID NO: 15 - protein, 13 aa, synthetic construct
PKAAYLQMNAAKG

SEQ ID NO: 16 - protein, 5 aa, synthetic construct
  source 1..5: note = reverse core or reverse modified peptideX2
NMQLY

SEQ ID NO: 17 - protein, 13 aa, synthetic construct
GKAANMQLYAAKG

SEQ ID NO: 18 - protein, 4 aa, synthetic construct
  source 1..4: note = modified peptideX2(4)
LQMN

SEQ ID NO: 19 - skipped

SEQ ID NO: 20 - protein, 13 aa, synthetic construct
GKVVYLQMNVVKG

SEQ ID NO: 21 - protein, 13 aa, synthetic construct
GKLLYLQMNLLKG

SEQ ID NO: 22 - protein, 9 aa, synthetic construct
GKYLQMNKG

SEQ ID NO: 23 - protein, 7 aa, synthetic construct
GYLQMNG

SEQ ID NO: 24 - protein, 15 aa, synthetic construct
GRSSAYLQMNASSRG

SEQ ID NO: 25 - protein, 15 aa, synthetic construct
GRSSANMQLYASSRG

(19) United States
(12) Patent Application Publication
(10) Pub. No.: US 2025/0382330 A1
(43) Pub. Date: Dec. 18, 2025

(54) IMMUNOLOGICALLY ACTIVE POLYPEPTIDE

(71) Applicant: CDRES PHARMA LLC, Idaho Falls, ID (US)

(72) Inventor: Craig D. Scoville, Idaho Falls, ID (US)

(21) Appl. No.: 18/878,452

(22) PCT Filed: Jun. 27, 2023
(86) PCT No.: PCT/US2023/069152

§ 371 Date: Dec. 23, 2024

Related U.S. Application Data
(60) Provisional application No. 63/356,867, filed on Jun. 29, 2022.

(51) Int. Cl. C07K 7/08; A61K 38/00; A61P 19/02; A61P 37/06

(52) U.S. Cl. CPC C07K 7/08; A61P 19/02; A61P 37/06; A61K 38/00

(57) ABSTRACT

Disclosed are immunomodulatory polypeptides that behave as weak human TLR2 agonists and as potent competitive antagonists of natural pathogenic ligands for TLR2. Also disclosed are compositions comprising such polypeptides, compositions comprising antibodies that specifically bind to such polypeptides, and methods of using the same, including for treating sepsis or reducing the severity or likelihood of occurrence of sepsis, in the treatment of inflammatory or autoimmune diseases including rheumatoid arthritis (RA), in the treatment of atherosclerosis, in the treatment of uveitis, in cancer treatment, in organ transplantation and for reducing graft rejection and promoting fertility. Pharmaceutical compositions and kits, and treatment methods are also disclosed.

### REFERENCE TO AN ELECTRONIC SEQUENCE LISTING

The contents of the electronic sequence listing (190141.405WO_SEQUENCE_LISTING.xml; Size: 27827 bytes; and Date of Creation: Jun. 25, 2023) is herein incorporated by reference in its entirety.

### BACKGROUND

### Technical Field

Embodiments of the present disclosure relate generally to immunomodulatory polypeptides, compositions comprising such polypeptides, and methods of using the same. More specifically, the present embodiments relate to short immunomodulatory polypeptides that specifically bind with low affinity to the human and murine toll-like receptors TLR2 and TLR4 such that they advantageously interfere with TLR2- and/or TLR4-mediated signaling.

### Description of the Related Art

### Sepsis, Inflammation and Immunity

The interaction between a host organism, such as a human, a mammal or another vertebrate animal, and a microbial pathogen such as a bacterium, virus, fungus or parasite, is complex and depends on genetic and environmental factors (e.g., nutrition, health, temperature); the interplay of these components may play a defining role in clinical outcome. One potential outcome of the host-pathogen interaction may be a subclinical host protective response by which the host eradicates the infection without any clinical evidence of infection, or the host and pathogen may engage in a battle that produces clinical symptoms but during which the host maintains control of the pathogen and eventually eradicates it. Alternatively, in sepsis a more intense battle may ensue that the pathogen and/or the host's own inflammatory response eventually overwhelms the host, resulting in death of the host.

Sepsis is a severe, systemic inflammatory condition that occurs in about 750,000 to 900,000 people each year in the United States, and approximately one-third of sepsis patients die (Kellum et al., 2007 Arch Intern Med 167 (15): 1655-1663). Published studies have concluded that severe sepsis is a common, expensive, and frequently fatal condition that deserves more universal attention (Decker, 2004 J. Clin Invest 113 (10): 1387-1389). Human sepsis may be identified in a patient when the patient meets at least two of the four criteria for Systemic Inflammatory Response Syndrome (SIRS) and demonstrates features of MODS (multiple organ dysfunction syndrome) (Remick, 2007 Am. J. Pathol. 170 (5): 1435-1444). The SIRS criteria include (1) body temperature>38° C. or <36° C., (2) heart rate>90 beats per minute (bpm), (3) respiratory rate>20 breaths per minute or arterial CO₂<32 mm Hg, and (4) circulating white blood cell (WBC) count>12,000/mm³ or <4000/mm³ or >10% immature forms (Remick 2007).

The innate immune system depends on the interplay between the pathogen associated molecular patterns (PAMPs) expressed by bacterial, viral, fungal and parasitic agents, and the pattern recognition receptors (PRRs) expressed by the cells of the host innate immune system, including macrophages, monocytes, neutrophils, and dendritic cells. Association of the immune cell surface receptor (PRR) and the pathogen cell surface ligand (PAMP) promotes relatively prompt phagocytosis of the pathogens by innate immune cells and triggers the expression by these host cells of the CD80 and CD86 surface proteins, which are important in recruiting the activation of the adaptive (acquired) immune system, over time, to generate an antigen-specific immune response.

Toll-like receptors (TLRs) are the PRRs of primary importance in higher mammals: in the human innate immune system there are at least ten different TLR polypeptides, while in mice there are at least 11 different TLRs (Rich, T., Toll and Toll-Like Receptors: An Immunologic Perspective. 2010 Kluwer Academic/Plenum Publishers, Dordrecht, NL; see also, e.g., Gorden et al., 2005 J. Immunol. 174:1259 and references cited therein). These type I integral membrane proteins associate with adaptor proteins and, when engaged by appropriate ligands, mediate signal transduction that results in activation of downstream transcription factors (e.g., NF-κB) to promote expression of cytokines, chemokines and other activation markers.

TLR2 is a TLR that binds primarily to potential pathogens such as Gram-positive bacteria, Mycobacteria, Borrelia (spirochetes), and yeast. TLR2 is present on the surfaces of certain host innate immune system cells, including myeloid lineage hematopoietic cells, as part of a heterodimer that may occur (in association with TLR1) as a TLR1/2 heterodimer, or (in association with TLR6) as a TLR2/6 heterodimer. Some innate immune system cells, including myeloid lineage hematopoietic cells, also express TLR4, which binds Gram negative bacteria and recognizes LPS and lipoteichoic acid. TLR4 occurs as a cell surface (TLR4/4) homodimer. The bacterial components recognized in binding interactions by TLR2 and TLR4 include peptidoglycan, lipoteichoic acid, and tripalmitoylated lipoproteins. Interference with TLR2/6 heterodimer assembly impaired TLR2-mediated sepsis without affecting TLR4-mediated sepsis (Fink et al., 2013 J. Immunol. 190:6410); hence, TLR2-mediated sepsis and TLR4-mediated sepsis can proceed via mechanisms that are independent of one another.

The pathophysiology of sepsis is complex but begins at a site of infection when a pathogen causes tissue injury, after first stimulating the innate immune system via TLRs and other PRRs, and subsequently provoking a response by the adaptive (acquired) immune system. Besides the pathogen-derived components (PAMPs) that are locally released early in this sequence of inflammatory events, accompanying tissue damage may also lead to the release of host cellular components that activate the innate immune response and are referred to as “danger associated molecular patterns,” or DAMPs. Examples of DAMPs include heat shock proteins (hsp) and alarmins, such as human mobility box group-1 protein. Coagulation factors, complement factors, mast cells, and platelets may all also play a role in this initial phase of localized tissue injury, which can lead to a local inflammatory response.

The hallmarks of inflammation include one or more of heat, redness, swelling and pain to which vasodilators are significant contributors, as mediated, for example, by bradykinins and histamine. The inflammatory response also involves the release of chemokines and activated complement factors that influence migration of neutrophils into the area of tissue injury and/or infection. Phagocytosis is enhanced by the interaction between PAMPs and PRRs on the surfaces of innate immune cells, with the subsequent release by these cells of pro-inflammatory cytokines such as tumor necrosis factor-alpha (TNFα), interleukin-1 (IL-1), interleukin-6 (IL-6), and interferon-gamma (IFNγ). The primary purpose of the localized inflammatory process is to contain and destroy the pathogen. However, if containment is not achieved and the pathogen is not eradicated, then the inflammatory process and the infection may spread. Once these events become systemic, the patient is at risk of developing sepsis (Hotchkiss et al., 2003 New Engl. J. Med. 348 (2): 138-150).

In systemic inflammation, the area of tissue injury that had previously been contained to a local milieu now instead sheds necrotic cellular components, pathogen and pathogen-derived debris, alarmins, PAMPs, and inflammatory cytokines into the circulation. The inflammatory process thus activates systemically many of the same innate immune system (e.g., PAMP-, DAMP- and PRR-driven) processes that were once localized. Global inflammatory phenomena characterize the resulting response, which includes systemic vasodilation, reduced vascular resistance, and increased cardiac output with tachycardia and tachypnea. The edema caused by vasodilation causes hypotension and hypoperfusion of critical organs. Ischemia in the organs promotes further tissue damage, which in turn further stimulates the inflammatory process. Concurrent with such widespread inflammatory events in sepsis, there is also evidence of immune suppression (manifest as lymphocyte anergy and the apoptosis-induced loss of CD4 T cells, B cells, and dendritic cells), along with reversible suspension of cellular functions in the organs (cell hibernation and cell stunning). These events contribute to secondary and nosocomial infections, complicating the host's status and further stimulating the inflammatory process (Fry, 2012, The American Surgeon 78:1-8).

Clinical trials of treating sepsis with various anti-inflammatory products such as corticosteroids, anti-endotoxin antibodies, anti-TNFα products, and IL-1 receptor antagonists, have been conducted without success (Hotchkiss et al., 2003 New Engl. J. Med. 348 (2): 138-150).

In a murine sepsis model, mice deficient for TLR2 or TLR2-derived signals were resistant to sepsis (Meng et al., 2004 J. Clin. Invest. 113 (10): 1473-1481). Meng et al. (2004) described a monoclonal antibody specific for the murine TLR2 extracellular domain that was able to block early events in a sepsis model system by interfering with the binding of a TLR2 agonist (P3CSK4, a mimetic of tripalmitoylated proteins found on bacterial surfaces) to TLR2.

Although the monoclonal antibody of Meng cross-reacts with human TLR2 (see also U.S. Pat. No. 8,623,353), this antibody is a murine immunoglobulin and requires specific binding interactions contributed by the immunoglobulin variable regions of two polypeptides (i.e., both the heavy and light chains). As such, problems associated with production of a protein macromolecule having six defined murine complementarity determining regions (CDRs) that originate in two distinct polypeptides (CDRs 1-3 from each of the immunoglobulin heavy and light chains) may hinder the development of a human therapeutic, such as proper folding and assembly of the variable regions, and removal of immunogenicity by engineering out murine determinants without compromising antigen binding specificity. Additionally, the rodent model may be limited in its predictive value for human sepsis, as noted by Decker (2004 J. Clin. Invest. 113:1387-1389).

A potential role for TLR2 in sepsis was also the subject of a report by Navarini et al. (2009 Proc. Nat. Acad. Sci. USA 106:7107-7112). These authors described experiments in which the TLR2 ligand Pam2Cys (5-(2,3-bis (palmitoyloxy) propyl) cysteine), a Mycoplasma-derived lipopeptide, elicited sepsis-like innate immune activation in a murine model of Listeria monocytogenes-driven neutrophil exhaustion. Co-administration to immunologically intact mice of an otherwise non-lethal (low) dose of Listeria along with Pam2Cys resulted in overwhelmingly lethal bacterial infections. Autopsy revealed depletion of neutrophils from bone marrow reservoirs (due to neutrophil migration) and the absence (due to neutrophil apoptosis) of live neutrophil infiltrates from tissue sites which, in experimental control animals that did not receive Pam2Cys, were abundant in neutrophils. Mice genetically deficient for TLR2 (tlr2−/−), by contrast, were free of disease when subject to the same inoculation regimen, a result the authors attributed to their lack of TLR2 receptors through which Pam2Cys could initiate the inflammatory cascade.

Use of a TLR2 ligand, Pam2Cys, thus detrimentally escalated innate immune activation that was instigated by a sub-lethal infection, causing it to progress to sepsis in much the same way as a lethal (high) Listeria dose: Neutrophils were driven to apoptosis without any apparent ability to forestall the immunosuppressive cytokine profile elaborated by phagocytes (macrophages and dendritic cells) involved in their clearance, thereby favoring an overwhelming bacterial infection (Roger et al., 2009 Proc. Nat. Acad. Sci. USA 106:6889-6890). Multiple additional reports describe significant neutrophil roles in the pathogenesis of sepsis, including exacerbation of disease by TLR2 activation (Navarini et al., 2009 Proc. Nat. Acad. Sci. USA 106:7107; Roger et al., 2009 Proc. Nat. Acad. Sci. USA 106:6889; Alves-Filho et al., 2009 Proc. Nat. Acad. Sci. USA 106:4018; Zou et al., 2011 Shock 36:370; Castoldi et al, 2012 PLOS ONE 7 (5): e37584; Pene et al., 2009 Infect. Immun. 77 (12): 5651).

Mice deficient for TLR4 or TLR4-derived signals were also sepsis-resistant, including experimental animals treated with an anti-TLR antibody (Roger et al., 2009 Proc. Nat. Acad. Sci. USA 106 (7): 2348) and animals that were genetically engineered to lack TLR2 and TLR4 (Castoldi et al, 2012 PLOS ONE 7 (5): e37584; Pene et al., 2009 Infect. Immun. 77 (12): 5651). The TLR4 antagonist Eritoran, however, failed to demonstrate efficacy for treating sepsis in a recent clinical study (Opal et al., 2013 J. Amer. Med. Assoc. 309 (11): 1154) despite previous encouraging reports for this agent (Tidswell et al., 2010 Crit. Care Med. 38:72; Barochia et al., 2011 Expert Opin. Drug Metab. Toxicol. 7 (4): 479), and the anti-TLR4 antibody described by Roger et al. (2009) had no effect on TLR2-mediated sepsis.

Previous work points to exacerbation of sepsis that results from activating TLR2 and/or TLR4 (Navarini et al., 2009 Proc. Nat. Acad. Sci. USA 106:7107; Roger et al., 2009 Proc. Nat. Acad. Sci. USA 106:6889; Alves-Filho et al., 2009 Proc. Nat. Acad. Sci. USA 106:4018; Zou et al., 2011 Shock 36:370; Castoldi et al, 2012 PLOS ONE 7 (5): e37584; Pene et al., 2009 Infect. Immun. 77 (12): 5651). Earlier efforts, however, have failed to arrive at an effective TLR2- and TLR4-directed immunomodulator for sepsis (Lorne et al., 2010 Intensive Care Med. 36:1826) that has a beneficial mechanism of action and favorable efficacy profile, and that further offers ease of manufacture and is nonimmunogenic in humans. More recently, an immunomodulatory polypeptide referred to as PeptideX2 has been shown to induce low-level elaboration of cytokines (including IL-6, IL-10, TNFα) by human peripheral white blood cells and thus to act as a weak TLR2/4 agonist in a sepsis-relevant model system by precluding the exuberant cytokine responses seen in sepsis (U.S. Pat. Nos. 10,365,275; 9,816,989; WO 2014/165282); this peptide has not, however, advanced to clinical development. In addition to TLR-mediated inflammatory mechanisms that may be induced by microbial pathogens, TLRs have also been implicated in other inflammatory contexts, including atherosclerosis and uveitis and also including autoimmune diseases such as rheumatoid arthritis and Crohn's disease. For instance, many autoimmune diseases are characterized by inappropriate and clinically deleterious inflammation, leading to harmful and in some cases irreversible tissue damage. Autoimmune arthritides represent an exemplary class of autoimmune inflammatory diseases, and may include but are not limited to rheumatoid arthritis, juvenile rheumatoid arthritis, ankylosing spondylitis, Reiter's syndrome, psoriatic arthritis, and lupus erythematosus (see, e.g., Gilliland et al., “Disorders of the joints and connective tissue,” Section 14, in Harrison's Principles of Internal Medicine, Eighth Ed., Thorn et al., eds. McGraw-Hill, New York, NY, 1977, pp. 2048-2080). Rheumatoid arthritis (RA) is one of the most prevalent arthritic disorders. RA, an autoimmune disorder, results in part from inflammation of the synovial membrane. In humans, peak onset of this disorder occurs in adults over 30 years of age (typically in their thirties and forties) and afflicts women three times more often than men. In extreme cases, chronic inflammation erodes and distorts the joint surfaces and connective tissue, resulting in severe articular deformity and constant pain. Moreover, RA often leads to osteoarthritis (OA), further compounding the destruction of the joint. The most common arthritic disorder, OA, is characterized by degenerative changes in the surface of articular cartilage. Alterations in the physicochemical structure of the cartilage make it less resistant to compressive and tensile forces. Finally, complete erosion occurs, leaving the subchondral bone exposed and susceptible to wear. Joints of the knees and hands are most often affected, as also may be one or more of the spine, hips, ankles and shoulders. In both RA and OA, degeneration of the weight bearing joints such as the hips and knees can be especially debilitating and often requires surgery to relieve pain and increase mobility. No means currently exist for halting or reversing the degenerative changes brought about by RA and related arthritic disorders. At the same time, over 40 million Americans seek symptomatic relief from arthritis in the form of prescription drugs. In such cases nonsteroidal, anti-inflammatory drugs (NSAIDS) are most often prescribed. While these compounds often alleviate or palliate the arthritic symptoms, they frequently have undesirable side effects, for example, nausea and gastrointestinal ulceration. Other compounds commonly prescribed for the treatment of arthritic disorders are the corticosteroids, such as triamcinolone, prednisolone and hydrocortisone. These drugs also have undesirable side effects, particularly where long term use may be required, and so may be contraindicated in many patients. In addition to difficulties in determining effective dosages, a number of adverse reactions have been reported during intra-articular treatment with these and other steroids. As a result, the use of corticosteroid treatments in the management of arthritic disorders is currently being reassessed.

TLR roles in the pathogenesis of RA, and TLRs as therapeutic targets in RA, have attracted attention in recent years. See, e.g., Lin et al., Clin Rev Allergy Immunol. 2014 October;47 (2): 136-47. doi: 10.1007/s12016-013-8402-y.PMID: 24352680; Luo et al., Endocr Metab Immune Disord Drug Targets. 2020; 20 (8): 1156-1165. doi: 10.2174/1871530320666200427115225. PMID: 32338225; Hennessy et al., Nat Rev Drug Discov. 2010 April;9 (4): 293-307. doi: 10.1038/nrd3203.PMID: 20380038; Sutmuller et al., Ann Rheum Dis. 2007 November;66 Suppl 3 (Suppl 3): iii91-5. doi: 10.1136/ard.2007.078535.PMID: 17934105; O'Neill et al., Pharmacol Rev. 2009 June;61 (2): 177-97. doi: 10.1124/pr.109.001073. Epub 2009 May 27.PMID: 19474110.

Clearly there remains a need for improved compositions and methods to treat, reduce the severity of, or reduce the likelihood of occurrence of TLR2- and/or TLR4-mediated sepsis and other inflammatory disorders, including atherosclerosis and uveitis and autoimmune diseases such as RA and Crohn's disease, as well as other inflammatory diseases, disorders, and conditions. Certain embodiments of the present disclosure address this need and provide other related advantages.

### Cancer

Cancer remains a devastating and largely intractable disease with significant unmet needs in the areas of patient treatment, clinical outcome and overall survival. The American Cancer Society (ACS) estimates that there will be over 1.6 million new cases of cancer diagnosed in the United States in 2012, not including non-invasive carcinoma in situ and also not including new cases of basal cell carcinoma and squamous cell skin cancer. The ACS also projects that there will be 577,190 cancer-related deaths in 2012, or an average of 1500 Americans dying each day from cancer, making cancer the second most common cause of death in the U.S., after heart disease. In 2007 the total medical cost of cancer in the U.S. was $226.8 billion, including direct medical costs for treatment of $103.8 billion and indirect costs due to lost productivity and premature death of $123 billion. The mean five-year survival rate overall for U.S. cancer patients has improved from 49% in 1975-1977 to 67% in 2001-2007. There clearly, however, still remains a pressing need for improved treatments and enhanced overall outcomes for cancer patients.

### Transplantation

Organ transplantation is often the best or only treatment option for end-stage organ failure, such as kidney disease, chronic conditions such as severe cirrhosis of the liver, and cancer, such as liver cancer, leukemias and lymphomas. Both solid organ and bone marrow transplants are performed to treat patients in need. It is estimated that about 100,000 solid organ transplants were performed worldwide in 2007, and of the roughly 30,000 bone marrow transplants performed annually, about 15,000 are allogenic transplants. Kidney, liver and heart transplants are the most common solid organ transplants.

The worldwide demand for donor tissues and organs far surpasses the supply, and there is a significant need in transplantation medicine to improve the availability of donor organs and minimize the long-term risk of rejection. For example, the World Health Organization (WHO) has estimated that only 10% of those in need of kidney transplants manage to get one. The National Kidney Foundation claims that about 18 patients die daily while waiting for a transplant of a vital organ such as a heart, liver, kidney, pancreas, lung, or bone marrow. Furthermore, the long-term graft survival rates for kidney and liver transplants are about 60-70% at 5-10 years post-transplant.

Rejection of the donor tissue by the host immune system is one of the largest problems faced in allogenic transplants. Despite donor-recipient human leukocyte antigen (HLA) histocompatibility matching and ABO blood group testing to provide matched donor tissue to the recipient, patients receiving transplants must undergo immunosuppressive treatment to prevent graft rejection or, in the case of bone marrow transplants, graft-versus-host disease (GVHD). Most immunosuppressors target T cells or cytokines secreted by T cells, and types of immunosuppressive agents currently used include monoclonal antibodies to lymphocytes and cytokine receptors (e.g., anti-IL-2Ra), calcineurin inhibitors (e.g., cyclosporine and tacrolimus), and cytokine receptor signal transduction inhibitors (e.g., sirolimus) (Chinen and Buckley, J Allergy Clin Immunol, 2010, 125 (2 Suppl 2): S324-S335). The downside to using immunosuppressive agents, sometimes over a course of several months, is that while protecting the graft from being rejected by the host immune system, or vice versa in the case of GVHD, they make the recipient especially vulnerable to infections and malignancies.

In addition, despite advances in immunosuppressive treatments which have significantly improved first year graft survival, long-term survival is still unsatisfactory. In a study of long-term kidney graft survival, Fernandex-Rodriguez et al (Transplantation Proceedings 2009 41 (6): 2357-2359) followed 1,029 first renal transplantations performed between November 1979 and December 2007, observed renal graft survival at 1, 5 and 10 years and correlated the results to the immunosuppressive therapy used, including azathioprine (AZA), cyclosporine (CsA), and tacrolimus (TAC). The findings indicated that graft survival rates at 5 and 10 years post-transplant were, respectively, 56% and 46% on AZA, 69% and 54% on CsA, and 77% and 60% on TAC. The study concluded that despite the decrease in acute rejection in kidney transplants, there was a significant decrease in renal graft survival after 12 months. Another study by Ruiz et al (Arch Surg 2006 141:735-742) reported liver graft survival at 1, 3 and 5 years post-transplant of 70%, 65% and 65%, respectively, and kidney graft survival at 1, 3 and 5 years post-transplant of 76%, 72% and 70%, respectively, thereby demonstrating that there is a significant decrease in graft survival following the first year.

As a semi-allograft, the maternal-host acceptance and tolerance of an embryo and placenta is similar in many ways to an allogeneic organ transplant. Implantation of the blastocyst into the uterine wall is a critical checkpoint for a successful pregnancy and results in the embryo adhering to the uterine lining and generating a vascular connection. Implantation failure can result in repeated miscarriages and failed in vitro fertilization (IVF) attempts. In particular, embryo implantation success rates in IVF patients vary widely, and success rates ranging from about 15% to about 30% have been reported (see, e.g., Croo et al. Human Reproduction, 15 (6): 1383-1388, 2000). Inflammation and the mother's immune response are believed to play a role in the successful implantation of an embryo.

Inflammation also plays a role later on in pregnancy, such as in preeclampsia and eclampsia, which affect an estimated 5-8% of all pregnancies and are the leading cause of maternal and fetal illness and mortality worldwide. Preeclampsia is characterized by high blood pressure and proteinuria, and if left unchecked, it can lead to the seizures of eclampsia. During pregnancy, the maternal adaptive immune response is down regulated, and the innate immune response is enhanced. However, the innate immunity also must be regulated, and it has been shown that neutrophils, non-antigen specific white blood cells of hematopoietic origin that are typically associated with inflammatory and anti-microbial responses, play a large role in preeclampsia (Cadden and Walsh, Hypertens Pregnancy (2008) 27 (4): 396-405).

Indeed, studies indicate that innate immune cells, such as neutrophils (often referred to as polymorphonuclear neutrophils, or PMN), are important in shaping, enhancing and regulating the adaptive immune response (see, e.g., Soehnlein, Trends in Immunology, 2009, 30 (11): 511-512 and Müller et al, Trends in Immunology, 2009, 30 (11): 522-530). Recent studies indicate that neutrophils may play a significant role in antitumor reactions (DiCarlo et al., 2001 Blood 97:339; Mumm et al., 2011 Canc. Cell 20:781) and in transplant rejection, in addition to the historical role of lymphocytes. In particular, Soo et al. (J. Heart and Lung Transplantation, 2009, 28 (11): 1198-1205) examined pre-operative neutrophil adhesion molecule expression after in vitro stimulation with LPS or PMA and then correlated these results with actual allograft success. Interestingly, pre-operative neutrophil surface CD11b expression after LPS stimulation correlated proportionally with the degree of rejection as detected in the first endomyocardial biopsy sample post heart transplantation, and the authors concluded “that neutrophils may contribute more to cardiac allograft rejection than previously thought.”

Another study identified increased IL-17 and neutrophilia in the broncho-alveolar lavage of patients undergoing acute lung transplant rejection (Chest 2007 131 (6): 1988-9). Furthermore, neutrophils have been shown to play a significant role in xenotransplantation rejection (Transplant Immunology 2009 21:70-74, Transplantation 2004 78:1721-1718), and means of attenuating neutrophil activity may play a significant role in improving the success of this type of transplantation approach. There is also increasing evidence that neutrophils can be activated to express MHC Class II molecules and develop antigen presenting cell (APC) characteristics and release cytokines such as IL-4, IL6, IL-10, IL-12, and TNFα and suppress T cell activity (e.g., Muller et al., 2009 Trends in Immunology 30 (11): 522-530, 2009; Vasconcelos et al., 2006 Blood 107:2192-2199; Rodriguez et al., 2009 Cancer Research 69:1553-1560, 2009. Therefore, the role of neutrophils may also be involved in allograft and xenograft acceptance.

Neutrophils are clearly of interest for research due to their role in inflammation and infection as well as other immune functions, including roles in pregnancy, transplantation and autoimmunity. A number of cell surface markers present on neutrophils have been utilized in an effort to characterize, identify and determine their activation state, such as CD64, CD11b, and CD83; however, few neutrophil markers exist that can readily be used to identify a distinct subset of neutrophils in the same way that other hematopoietic cell surface markers can be used to categorize adaptive immune cells, for instance, according to maturational state, differentiation lineage, and functional properties (e.g., CD4 versus CD8 T lymphocytes, surface immunoglobulin (sIg) changes in B lymphocyte differentiation and maturation, and other regulatory cell markers, e.g., CD45 isoforms, distinct integrin a and chain heterodimer expression, etc.). Accordingly, a need exists for more refined neutrophil markers in order to functionally characterize these cells. (See, e.g., Mason et al., (Eds.), Leukocyte Typing VII, 2002 Oxford Univ. Press, USA.)

An increased understanding of the mechanisms resulting in graft rejection have led to advancements in the availability and mechanistic understanding of immunosuppressants; however, there is still a significant medical need for immunosuppressive agents capable of improving the acceptance of donor organs and minimizing the risk of rejection while at the same time placing recipients at a lesser risk of infections and malignancies. In particular, there is an unmet need for treatments that improve long term graft survival and also improve the success of xenotransplantation. In addition, immunosuppressive agents that are useful for conception, providing improved rates of implantation and a reduced risk of multiples, as well as preventing conditions like preeclampsia, are needed.

The compositions and methods of the present disclosure address the needs described above and offer other related advantages.

### BRIEF SUMMARY

In one aspect of the present disclosure, there is provided an isolated immunomodulatory polypeptide that is:
(a) no more than 31, 30, 29, 28, 27, 26, 25, 24, 23, 22, 21, 20, 19, 18, 17, 16, 15, 14, 13, 12, 11, 10, or 9 amino acids in length, comprising or consisting of the amino acid sequence X₁X₂X₃X₄YLQMNX₅X₆X₇X₈ as set forth in SEQ ID NO: 11, wherein X₁ and X₈ are each independently glycine (G) or proline (P), X₂ and X₇ are each independently lysine (K) or arginine (R), and X₃, X₄, X₅ and X₆ are each independently alanine (A) or nothing,
(b) YLQMN as set forth in SEQ ID NO:5,
(c) NMQLY as set forth in SEQ ID NO:16, or
(d) no more than 31, 30, 29, 28, 27, 26, 25, 24, 23, 22, 21, 20, 19, 18, 17, 16, 15, 14, or 13 amino acids in length, comprising or consisting of the amino acid sequence GKAANMQLYAAKG as set forth in SEQ ID NO:17.

In certain embodiments, the immunomodulatory polypeptides are no more than 31, 30, 29, 28, 27, 26, 25, 24, 23, 22, 21, 20, 19, 18, 17, 16, 15, 14, or 13 amino acids in length, and comprise the amino acid sequence of general formula: G-X₂-AAYLQMNAA-X₇-G as set forth in SEQ ID NO: 4 wherein X₁ and X₂ are independently selected from K and R, for example, GRAAYLQMNAARG (SEQ ID NO: 10).

In certain embodiments of this aspect, the amino acid sequence as set forth in SEQ ID NO: 11 is selected from the group consisting of:
(a) GKAAYLQMNAAKG as set forth in SEQ ID NO: 3,
(b) GRAAYLQMNAARG as set forth in SEQ ID NO: 10,
(c) PKAAYLQMNAAKP as set forth in SEQ ID NO: 12,
(d) GKAYLQMNAKG as set forth in SEQ ID NO: 13,
(e) GKAAYLQMNAAKP as set forth in SEQ ID NO: 14,
(f) PKAAYLQMNAAKG as set forth in SEQ ID NO: 15, and
(g) GKYLQMNKG as set forth in SEQ ID NO: 22.

In certain embodiments, the immunomodulatory polypeptide is YLQMN as set forth in SEQ ID NO:5.

In certain other embodiments, the immunomodulatory polypeptide is NMQLY as set forth in SEQ ID NO:16.

In another aspect, there is provided a pharmaceutical composition, comprising (a) any of the above-described immunomodulatory polypeptides and (b) a physiologically acceptable carrier.

In a further aspect, there is provided a fusion protein comprising any of the above-described immunomodulatory polypeptides fused to a fusion polypeptide domain; in certain further embodiments there is provided a pharmaceutical composition comprising the fusion protein of; and a physiologically acceptable carrier.

In another aspect, there is provided an immunomodulatory polypeptide for use as a medicament, wherein the immunomodulatory polypeptide is
(a) no more than 31, 30, 29, 28, 27, 26, 25, 24, 23, 22, 21, 20, 19, 18, 17, 16, 15, 14, 13, 12, 11, 10, or 9 amino acids in length, comprising or consisting of the amino acid sequence X₁X₂X₃X₄YLQMNX₅X₆X₇X₈ as set forth in SEQ ID NO: 11, wherein X₁ and X₈ are each independently glycine (G) or proline (P), X₂ and X₇ are each independently lysine (K) or arginine (R), and X₃, X₄, X₅ and X₆ are each independently alanine (A) or nothing,
(b) YLQMN as set forth in SEQ ID NO:5,
(c) NMQLY as set forth in SEQ ID NO:16, or
(d) no more than 31, 30, 29, 28, 27, 26, 25, 24, 23, 22, 21, 20, 19, 18, 17, 16, 15, 14, or 13 amino acids in length, comprising or consisting of the amino acid sequence GKAANMQLYAAKG as set forth in SEQ ID NO:17.

In certain embodiments of this aspect, the amino acid sequence as set forth in SEQ ID NO: 11 is selected from the group consisting of:
(a) GKAAYLQMNAAKG as set forth in SEQ ID NO: 3,
(b) GRAAYLQMNAARG as set forth in SEQ ID NO: 10,
(c) PKAAYLQMNAAKP as set forth in SEQ ID NO: 12,
(d) GKAYLQMNAKG as set forth in SEQ ID NO: 13,
(e) GKAAYLQMNAAKP as set forth in SEQ ID NO: 14,
(f) PKAAYLQMNAAKG as set forth in SEQ ID NO: 15, and
(g) GKYLQMNKG as set forth in SEQ ID NO: 22.

In another aspect, there is provided an immunomodulatory polypeptide for use in the treatment of TLR2-mediated sepsis and/or TLR4-mediated sepsis, wherein the immunomodulatory polypeptide is
(a) no more than 31, 30, 29, 28, 27, 26, 25, 24, 23, 22, 21, 20, 19, 18, 17, 16, 15, 14, 13, 12, 11, 10, or 9 amino acids in length, comprising or consisting of the amino acid sequence X₁X₂X₃X₄YLQMNX₅X₆X₇X₈ as set forth in SEQ ID NO: 11, wherein X₁ and X₈ are each independently glycine (G) or proline (P), X₂ and X₇ are each independently lysine (K) or arginine (R), and X₃, X₄, X₅ and X₆ are each independently alanine (A) or nothing,
(b) YLQMN as set forth in SEQ ID NO:5,
(c) NMQLY as set forth in SEQ ID NO:16, or
(d) no more than 31, 30, 29, 28, 27, 26, 25, 24, 23, 22, 21, 20, 19, 18, 17, 16, 15, 14, or 13 amino acids in length, comprising or consisting of the amino acid sequence GKAANMQLYAAKG as set forth in SEQ ID NO:17.

In certain embodiments of this aspect, the amino acid sequence as set forth in SEQ ID NO: 11 is selected from the group consisting of:
(a) GKAAYLQMNAAKG as set forth in SEQ ID NO: 3,
(b) GRAAYLQMNAARG as set forth in SEQ ID NO: 10,
(c) PKAAYLQMNAAKP as set forth in SEQ ID NO: 12,
(d) GKAYLQMNAKG as set forth in SEQ ID NO: 13,
(e) GKAAYLQMNAAKP as set forth in SEQ ID NO: 14,
(f) PKAAYLQMNAAKG as set forth in SEQ ID NO: 15, and
(g) GKYLQMNKG as set forth in SEQ ID NO: 22.

In another aspect, there is provided an immunomodulatory polypeptide for use in the treatment of one or more of conditions (a)-(l), wherein the immunomodulatory polypeptide is (a) no more than 31, 30, 29, 28, 27, 26, 25, 24, 23, 22, 21, 20, 19, 18, 17, 16, 15, 14, 13, 12, 11, 10, or 9 amino acids in length, comprising or consisting of the amino acid sequence

X₁X₂X₃X₄YLQMNX₅X₆X₇X₈ as set forth in SEQ ID NO: 11, wherein X₁ and X₈ are each independently glycine (G) or proline (P), X₂ and X₇ are each independently lysine (K) or arginine (R), and X₃, X₄, X₅ and X₆ are each independently alanine (A) or nothing,
(b) YLQMN as set forth in SEQ ID NO:5,
(c) NMQLY as set forth in SEQ ID NO: 16, or
(d) no more than 31, 30, 29, 28, 27, 26, 25, 24, 23, 22, 21, 20, 19, 18, 17, 16, 15, 14, or 13 amino acids in length, comprising or consisting of the amino acid sequence GKAANMQLYAAKG as set forth in SEQ ID NO:17.

wherein the condition is selected from: (a) graft rejection to be decreased in a graft transplant recipient, (b) the graft rejection of (a) wherein the graft transplant is selected from kidney, heart, liver, pancreas and lung, (c) graft versus host disease in a bone marrow transplant recipient, (d) preeclampsia or hemolysis-elevated liver enzymes-low platelet count (HELLP) syndrome, (e) severe preeclampsia, (f) rheumatoid arthritis, (g) a malignant condition, (h) the malignant condition of (g) which is selected from breast cancer, ovarian cancer, adenoma, colorectal carcinoma, gastric carcinoma, lung carcinoma, prostate carcinoma, hepatocellular carcinoma, melanoma, leukemia and lymphoma, (i) an autoimmune disease, (j) the autoimmune disease of (i) which is selected from rheumatoid arthritis, psoriatic arthritis, ulcerative colitis, Crohn's disease, seronegative spondyloarthopathies, systemic lupus erythematosus, Behcet's disease and vasculitis, (k) atherosclerosis, and (1) uveitis.

In certain embodiments of this aspect, the amino acid sequence as set forth in SEQ ID NO: 11 is selected from the group consisting of:
(a) GKAAYLQMNAAKG as set forth in SEQ ID NO: 3,
(b) GRAAYLQMNAARG as set forth in SEQ ID NO: 10,
(c) PKAAYLQMNAAKP as set forth in SEQ ID NO: 12,
(d) GKAYLQMNAKG as set forth in SEQ ID NO: 13,
(e) GKAAYLQMNAAKP as set forth in SEQ ID NO: 14,
(f) PKAAYLQMNAAKG as set forth in SEQ ID NO: 15, and
(g) GKYLQMNKG as set forth in SEQ ID NO: 22.

In another embodiment, there is provided a method of inducing a peripheral blood white cell response that includes cellular release of at least one of IL-6, IL-10 and TNFα, comprising contacting one or a plurality of peripheral blood white cells in vitro or in vivo with the herein described immunomodulatory polypeptide, under conditions and for a time sufficient to induce detectable cellular release of at least one of IL-6, IL-10 and TNFα.

In another embodiment there is provided a method of treating an organ to be transplanted into an allogeneic recipient to reduce a likelihood or severity of allograft rejection by the recipient, comprising contacting the organ with the herein described immunomodulatory polypeptide, under conditions and for a time sufficient to reduce the likelihood or severity of allograft rejection.

In another embodiment there is provided a method of selectively labeling a mammalian peripheral blood white cell neutrophil subpopulation, comprising contacting a population of mammalian peripheral blood white cells which comprises neutrophils with the herein described immunomodulatory polypeptide, wherein either (i) the immunomodulatory polypeptide comprises a detectable label or (ii) the immunomodulatory polypeptide is indirectly detected. In certain further embodiments, the detectable label is selected from a fluorescent dye, a radioactive substance and a metal particle.

Turning to another aspect, there is provided a method for treating, reducing severity of, or reducing likelihood of occurrence of TLR2-mediated sepsis and/or TLR4-mediated sepsis in a subject, comprising administering to the subject a therapeutically effective amount of an immunomodulatory polypeptide that is
(a) no more than 31, 30, 29, 28, 27, 26, 25, 24, 23, 22, 21, 20, 19, 18, 17, 16, 15, 14, 13, 12, 11, 10, or 9 amino acids in length, comprising or consisting of the amino acid sequence X₁X₂X₃X₄YLQMNX₅X₆X₇X₈ as set forth in SEQ ID NO: 11, wherein X₁ and X₈ are each independently glycine (G) or proline (P), X₂ and X₇ are each independently lysine (K) or arginine (R), and X₃, X₄, X₅ and X₆ are each independently alanine (A) or nothing,
(b) YLQMN as set forth in SEQ ID NO:5,
(c) NMQLY as set forth in SEQ ID NO:16, or
(d) no more than 31, 30, 29, 28, 27, 26, 25, 24, 23, 22, 21, 20, 19, 18, 17, 16, 15, 14, or 13 amino acids in length, comprising or consisting of the amino acid sequence

GKAANMQLYAAKG as set forth in SEQ ID NO:17.

In certain embodiments of this aspect, the amino acid sequence as set forth in SEQ ID NO: 11 is selected from the group consisting of:
(a) GKAAYLQMNAAKG as set forth in SEQ ID NO: 3,
(b) GRAAYLQMNAARG as set forth in SEQ ID NO: 10,
(c) PKAAYLQMNAAKP as set forth in SEQ ID NO: 12,
(d) GKAYLQMNAKG as set forth in SEQ ID NO: 13,
(e) GKAAYLQMNAAKP as set forth in SEQ ID NO: 14,
(f) PKAAYLQMNAAKG as set forth in SEQ ID NO: 15, and
(g) GKYLQMNKG as set forth in SEQ ID NO: 22.

In another aspect, there is provided a method of treating a patient, comprising administering to the patient a therapeutically effective amount of an immunomodulatory polypeptide that is
(a) no more than 31, 30, 29, 28, 27, 26, 25, 24, 23, 22, 21, 20, 19, 18, 17, 16, 15, 14, 13, 12, 11, 10, or 9 amino acids in length, comprising or consisting of the amino acid sequence X₁X₂X₃X₄YLQMNX₅X₆X₇X₈ as set forth in SEQ ID NO: 11, wherein X₁ and X₈ are each independently glycine (G) or proline (P), X₂ and X₇ are each independently lysine (K) or arginine (R), and X₃, X₄, X₅ and X₆ are each independently alanine (A) or nothing,
(b) YLQMN as set forth in SEQ ID NO:5,
(c) NMQLY as set forth in SEQ ID NO:16, or
(d) no more than 31, 30, 29, 28, 27, 26, 25, 24, 23, 22, 21, 20, 19, 18, 17, 16, 15, 14, or 13 amino acids in length, comprising or consisting of the amino acid sequence

GKAANMQLYAAKG as set forth in SEQ ID NO:17, wherein the method is selected from: (a) a method for treating, reducing severity of, or reducing likelihood of occurrence of TLR2-mediated sepsis in the patient, (b) a method of decreasing graft rejection wherein the patient is a transplant patient, (c) the method of (b) wherein the transplant is selected from kidney, heart, liver, pancreas and lung, (d) a method of treating or decreasing graft versus host disease wherein the patient is a bone marrow transplant patient, (e) a method of treating preeclampsia or hemolysis-elevated liver enzymes-low platelet count (HELLP) syndrome in the patient, (f) the method of (e) wherein the patient has been diagnosed with severe preeclampsia, (g) a method of treating rheumatoid arthritis in the patient, (h) a method of treating a malignant condition in the patient, (i) the method of (h) wherein treating the malignant condition comprises at least one of killing a tumor cell and inhibiting metastasis, (j) the method of (h) wherein the malignant condition is selected from breast cancer, ovarian cancer, adenoma, colorectal carcinoma, gastric carcinoma, lung carcinoma, prostate carcinoma, hepatocellular carcinoma, melanoma, leukemia and lymphoma, (k) a method of treating an autoimmune disease in the patient, (1) the method of (k) wherein the autoimmune disease is selected from rheumatoid arthritis, psoriatic arthritis, ulcerative colitis, Crohn's disease, seronegative spondyloarthopathies, systemic lupus erythematosus, Behcet's disease and vasculitis, (m) a method of treating atherosclerosis, and (n) a method of treating uveitis.

In certain embodiments of this aspect, the amino acid sequence as set forth in SEQ ID NO: 11 is selected from the group consisting of:
(a) GKAAYLQMNAAKG as set forth in SEQ ID NO: 3,
(b) GRAAYLQMNAARG as set forth in SEQ ID NO: 10,
(c) PKAAYLQMNAAKP as set forth in SEQ ID NO: 12,
(d) GKAYLQMNAKG as set forth in SEQ ID NO: 13,
(e) GKAAYLQMNAAKP as set forth in SEQ ID NO: 14,
(f) PKAAYLQMNAAKG as set forth in SEQ ID NO: 15, and
(g) GKYLQMNKG as set forth in SEQ ID NO: 22.

In certain compositions and methods according to the herein described embodiments, the immunomodulatory polypeptide comprises or consists of a polypeptide of no more than 28, 27, 26, 25, 24, 23, 22, 21, 20, 19, 18, 17, 16, 15, 14, or 13 amino acids which comprises the amino acid sequence GKAAYLQMNAAKG as set forth in SEQ ID NO: 3. In certain compositions and methods according to the herein described embodiments, the immunomodulatory polypeptide comprises or consists of a polypeptide of no more than 28, 27, 26, 25, 24, 23, 22, 21, 20, 19, 18, 17, 16, 15, 14, or 13 amino acids which comprises the amino acid sequence as set forth in SEQ ID NO: 10, 12, 13, 14, 15 or 17. In certain compositions and methods according to the herein described embodiments, the immunomodulatory polypeptide consists of YLQMN as set forth in SEQ ID NO:5.

In certain compositions and methods according to the herein described embodiments, the immunomodulatory polypeptide consists of NMQLY as set forth in SEQ ID NO: 16.

In another aspect, there is provided an isolated polynucleotide comprising a nucleic acid sequence that encodes the immunomodulatory polypeptide described herein. In a related aspect, there is provided an expression vector comprising the polynucleotide. In a further aspect, there is provided a host cell transformed or transfected with the expression vector.

In another embodiment there is provided a method of producing an immunomodulatory polypeptide that is
(a) no more than 31, 30, 29, 28, 27, 26, 25, 24, 23, 22, 21, 20, 19, 18, 17, 16, 15, 14, 13, 12, 11, 10, or 9 amino acids in length, comprising or consisting of the amino acid sequence X₁X₂X₃X₄YLQMNX₅X₆X₇X₈ as set forth in SEQ ID NO: 11, wherein X₁ and X₈ are each independently glycine (G) or proline (P), X₂ and X₇ are each independently lysine (K) or arginine (R), and X₃, X₄, X₅ and X₆ are each independently alanine (A) or nothing,
(b) YLQMN as set forth in SEQ ID NO:5,
(c) NMQLY as set forth in SEQ ID NO:16, or
(d) no more than 31, 30, 29, 28, 27, 26, 25, 24, 23, 22, 21, 20, 19, 18, 17, 16, 15, 14, or 13 amino acids in length, comprising or consisting of the amino acid sequence

GKAANMQLYAAKG as set forth in SEQ ID NO:17, said method comprising the steps of: a) culturing the above described host cell under conditions and for a time sufficient to permit expression of the immunomodulatory polypeptide; and b) isolating the immunomodulatory polypeptide from the cultured host cell.

In another aspect, there is provided an isolated polynucleotide that is selected from: (a) an isolated antisense polynucleotide comprising a nucleic acid sequence that is complementary to the above described immunomodulatory polypeptide-encoding polynucleotide, (b) an isolated small interfering RNA (siRNA) polynucleotide that is capable of substantially silencing, and is complementary to a region of at least 18 and no more than 69 contiguous nucleotides in, a nucleic acid which encodes the above described immunomodulatory polypeptide, and (c) an isolated ribozyme that specifically binds to the above described immunomodulatory polypeptide-encoding polynucleotide.

In another aspect, there is provided an isolated antibody, or antigen-binding fragment thereof, that specifically binds to the above described and herein disclosed immunomodulatory polypeptide. In certain preferred embodiments the antibody is a polyclonal antibody. In certain embodiments, there is provided a pharmaceutical composition comprising the antibody that specifically binds to the above described and herein disclosed immunomodulatory polypeptide; and a physiologically acceptable carrier.

In another aspect, there is provided a method for detecting, in a biological sample, an immunomodulatory polypeptide that comprises the above described and herein disclosed immunomodulatory, said method comprising the steps of: (a) contacting the biological sample with an antibody that specifically binds said immunomodulatory polypeptide, or an antigen-binding fragment of said antibody, under conditions and for a time sufficient for specific antibody binding to the immunomodulatory polypeptide to take place; and (b) detecting specific binding of the antibody to the immunomodulatory peptide, and thereby detecting the immunomodulatory peptide in the sample. In certain further embodiments, at least one of: (i) the antibody is a polyclonal antibody, (ii) the antibody is linked to a support material, (iii) the antibody is linked to a detectable label, or (iv) the biological sample is obtained from a subject that is selected from a human, a non-human primate, a non-primate mammal, a non-mammalian vertebrate, an invertebrate eukaryote and a prokaryote.

In another aspect, there is provided a method of promoting implantation of an embryo in a pregnant or pseudopregnant mammal, comprising contacting at least one of the embryo and the pregnant or pseudopregnant mammal with an immunomodulatory polypeptide that is
(a) no more than 31, 30, 29, 28, 27, 26, 25, 24, 23, 22, 21, 20, 19, 18, 17, 16, 15, 14, 13, 12, 11, 10, or 9 amino acids in length, comprising or consisting of the amino acid sequence X₁X₂X₃X₄YLQMNX₅X₆X₇X₈ as set forth in SEQ ID NO: 11, wherein X₁ and X₈ are each independently glycine (G) or proline (P), X₂ and X₇ are each independently lysine (K) or arginine (R), and X₃, X₄, X₅ and X₆ are each independently alanine (A) or nothing,
(b) YLQMN as set forth in SEQ ID NO:5,
(c) NMQLY as set forth in SEQ ID NO:16, or
(d) no more than 31, 30, 29, 28, 27, 26, 25, 24, 23, 22, 21, 20, 19, 18, 17, 16, 15, 14, or 13 amino acids in length, comprising or consisting of the amino acid sequence GKAANMQLYAAKG as set forth in SEQ ID NO:17, under conditions and for a time sufficient to promote embryonic implantation. In certain further embodiments, the pregnant or pseudopregnant mammal is a human, and in certain still further embodiments the embryo is produced by in vitro fertilization.

In another aspect, there is provided a method for detecting, in a biological sample that comprises one or a plurality of nucleic acid molecules, expression of a polynucleotide that encodes an immunomodulatory polypeptide that is
(a) no more than 31, 30, 29, 28, 27, 26, 25, 24, 23, 22, 21, 20, 19, 18, 17, 16, 15, 14, 13, 12, 11, 10, or 9 amino acids in length, comprising or consisting of the amino acid sequence

X₁X₂X₃X₄YLQMNX₅X₆X₇X₈ as set forth in SEQ ID NO: 11, wherein X₁ and X₈ are each independently glycine (G) or proline (P), X₂ and X₇ are each independently lysine (K) or arginine (R), and X₃, X₄, X₅ and X₆ are each independently alanine (A) or nothing,
(b) YLQMN as set forth in SEQ ID NO:5,
(c) NMQLY as set forth in SEQ ID NO:16, or
(d) no more than 31, 30, 29, 28, 27, 26, 25, 24, 23, 22, 21, 20, 19, 18, 17, 16, 15, 14, or 13 amino acids in length, comprising or consisting of the amino acid sequence GKAANMQLYAAKG as set forth in SEQ ID NO:17, said method comprising the steps of: (a) contacting the sample with at least one of (i) the above described and herein disclosed antisense polynucleotide, and (ii) the above described and herein disclosed polynucleotide, under conditions and for a time sufficient for specific nucleic acid hybridization to occur; and (b) detecting specific hybridization of at least one nucleic acid of the sample to at least one of said antisense polynucleotide and said polynucleotide, and thereby detecting, in the sample, expression of the polynucleotide that encodes the immunomodulatory peptide. In certain further embodiments, the biological sample is obtained from a subject that is selected from the group consisting of a human, a non-human primate, a non-primate mammal, a non-mammalian vertebrate, an invertebrate eukaryote and a prokaryote.

In another aspect, there is provided an isolated immunomodulatory polypeptide that competes with PeptideX2-13 or a variant thereof for specific binding to a human neutrophil, wherein said PeptideX2-13 comprises the amino acid sequence GKAAYLQMNAAKG as set forth in SEQ ID NO: 3 and wherein said variant thereof comprises or consists of
(a) the amino acid sequence of general formula X₁X₂X₃X₄YLQMNX₅X₆X₇X₈ as set forth in SEQ ID NO: 11, wherein X₁ and X₈ are each independently glycine (G) or proline (P), X₂ and X₇ are each independently lysine (K) or arginine (R), and X₃, X₄, X₅ and X₆ are each independently alanine (A) or nothing,
(b) YLQMN as set forth in SEQ ID NO:5,
(c) NMQLY as set forth in SEQ ID NO: 16, or
(d) GKAANMQLYAAKG as set forth in SEQ ID NO:17.

In certain further aspect, the immunomodulatory polypeptide that competes with PeptideX2-13 or a variant thereof for specific binding to a human neutrophil interferes with PeptideX2-13 binding to either or both of human TLR2 and human TLR4.

These and other aspects and embodiments of the present disclosure will become apparent upon reference to the following detailed description and attached drawings. All references disclosed herein are hereby incorporated by reference in their entirety as if each was incorporated individually.

### BRIEF DESCRIPTION OF THE DRAWINGS

FIG. 1 shows results from an in vivo study of the effects of PeptideX2 (SEQ ID NO: 1) in collagen-induced arthritis in Lewis rats as an animal model of rheumatoid arthritis. Administration of PeptideX2 (SEQ ID NO: 1) at 200 μg/ml (open circles) decreased the arthritis index in the experimental animals while the vehicle control (solid squares) had no effect.

FIG. 2 shows the effects of non-self-aggregating G2-peptideX2 (GKSIAYLQMNSLKG, SEQ ID NO: 2) at 100 μg/ml, 250 μg/ml, and 500 μg/ml on TLR2-driven NF-κB activation in HEK293 cell lines transfected with human TLR2 (hTLR2, InvivoGen, San Diego, CA). HEK293 cells (50,000 to 75,000 cells/well) expressing TLR2 were plated in wells of a 96-well plate (200 μl total volume) containing either G2-PeptideX2 (in 20 μl buffer), or buffer only (20 μl) as a vehicle control. All cells contained a reporter construct having coding sequence for Secreted Embryonic Alkaline Phosphatase (SEAP) under the control of a promoter inducible by the transcription of NF-κB, and were incubated in media containing a detectable SEAP expression indicator (InvivoGen, San Diego, CA). After 16-20 hours incubation at 37° C., the optical density of culture supernatants was read at 650 nm on a Molecular Devices SpectraMax™ 340PC absorbance detector. Not shown: Similar levels of hTLR2-driven NF-κB activation were observed in response to peptideX2 (KSIAYLQMNSLK, SEQ ID NO: 1) and in response to the PeptideX2-derived pentameric peptide referred to herein as “pentide” (YLQMN, SEQ ID NO: 5).

FIG. 3 shows results from an in vivo study of the effects of non-self-aggregating PeptideX2-13 (SEQ ID NO: 3) in collagen-induced arthritis in Lewis rats as an animal model of rheumatoid arthritis. Eleven Lewis rats were prepared using the collagen induced arthritis protocol. At day 15 thru day 30, three rats were given daily dosing of PeptideX2-13 (SEQ ID NO: 3) at 100 μg/ml, three rats were given daily dosing of PeptideX2-13 (SEQ ID NO: 3) at 250 μg/ml, and three rats were given daily dosing of PeptideX2-13 (SEQ ID NO: 3) at 500 μg/ml; as a positive control group, two rats were given prednisolone at 10 mg/kg/day. No adverse effects were observed in any of the animals. Administration of PeptideX2-13 (SEQ ID NO: 3) at 250 μg/ml (solid diamonds) and 500 μg/ml (solid circles) decreased the arthritis index in the experimental animals while the lowest dosage of 100 μg/ml (open squares) had minimal effect. Average arthritic index was also decreased by the positive control anti-inflammatory corticosteroid compound, prednisolone (solid triangles).

FIG. 4 shows the effects of soluble non-self-aggregating peptideX2-13 (SEQ ID NO: 3, GKAAYLQMNAAKG, Lane 1), soluble non-self-aggregating K2R/K12R-substituted peptide X₂-13, SEQ ID NO: 10, GRAAYLQMNAARG, Lane 2), insoluble aggregates of modified peptidex2-13 in which amino acids at positions 3, 4, 10 and 11 were substituted with nonpolar amino acids (SEQ ID NOS: 20 and 21) (FIG. 4, lanes 3-4), and soluble variants of peptidex2-13 in which glycine at positions 1 and 13 was replaced with proline (SEQ ID NO: 12) (FIG. 4, lane 5) or in which the amino acids at positions 3, 4, 10, and 11 were deleted (SEQ ID NO: 22) (FIG. 4, lane 6) at 500 μg/ml on TLR2-driven NF-κB activation in HEK293 cell lines transfected with human TLR2 (hTLR2, InvivoGen, San Diego, CA). HEK293 cells (50,000 to 75,000 cells/well) expressing TLR2 were plated in wells of a 96-well plate (200 μl total volume) containing PeptideX2-13 or modified PeptideX2-13 (in 20 μl buffer). All cells contained a reporter construct having coding sequence for Secreted Embryonic Alkaline Phosphatase (SEAP) under the control of a promoter inducible by the transcription of NF-κB and were incubated in media containing a detectable SEAP expression indicator (InvivoGen, San Diego, CA). After 16-20 hours incubation at 37° C., the optical density of culture supernatants was read at 650 nm on a Molecular Devices SpectraMax™ 340PC absorbance detector.

FIG. 5 shows the effects of pentide (YLQMN, SEQ ID NO:5) at 200 μg/ml on NF-κB activation in HEK293 cell lines transfected with human TLR2, 3, 4, 5, 7, 8 or 9. Pentide (YLQMN, SEQ ID NO:5) at 200 μg/ml was tested against human TLR panel consisting of TLR2, 3, 4, 5, 7, 8, and 9 cell lines. Experiments were performed by InvivoGen, San Diego, CA, by assessing NF-κB activation in HEK293 cells expressing a given human TLR. Results shown reveal values minus controls and were done in triplicate. No evidence of any activity was observed except for slight stimulatory effect on human TLR2.

FIG. 6 shows the effects of PeptideX2 (KSIAYLQMNSLK, SEQ ID NO:1), pentide (YLQMN, SEQ ID NO:5, “m-peptideX2”), reverse modified peptideX2 (NMQLY, SEQ ID NO: 16, “r-peptideX2”), modified peptideX2 (4) (LQMN, SEQ ID NO:18, “m-peptideX2 (4)”), and modified peptideX2 (3) (QMN, SEQ ID NO:19, “m-peptideX2 (3)”) at 200 μg/ml on human NF-κB activation in HEK293 cell lines transfected with human TLR2, 3, 4, 5, 7, 8 or 9. PeptideX2 (KSIAYLQMNSLK, SEQ ID NO:1), pentide (YLQMN, SEQ ID NO:5), reverse modified peptideX2 (NMQLY, SEQ ID NO:16), modified peptideX2 (4) (LQMN, SEQ ID NO: 18), and modified peptideX2 (3) (QMN, SEQ ID NO:19) at 200 μg/ml were all tested at 200 μg/ml tested against human TLR panel consisting of TLR2, 3, 4, 5, 7, 8, and 9 cell lines. Experiments performed by InvivoGen, San Diego, CA, by assessing NF-κB activation in HEK293 cells expressing a given human TLR. Results shown reveal values minus controls and were done in triplicate. Results show that activity of peptideX2 was greater than reverse modified peptideX2 with TLR2 and negligible activity with other TLRs.

FIG. 7 shows the effects of PeptideX2 (KSIAYLQMNSLK, SEQ ID NO:1), pentide (YLQMN, SEQ ID NO:5, “mod-peptideX2”), and reverse modified peptideX2 (NMQLY, SEQ ID NO: 16, “rev-peptideX2”) at 200 μg/ml on human NF-κB activation in HEK293 cell lines transfected with human TLR2, 3, 4, 5, 7, 8 or 9. PeptideX2 (KSIAYLQMNSLK, SEQ ID NO: 1), pentide (YLQMN), and reverse modified peptideX2 (NMQLY) were all tested at 200 μg/ml against human TLR panel consisting of TLR2, 3, 4, 5, 7, 8, and 9 cell lines. Experiments performed by InvivoGen, San Diego, CA, by assessing NF-κB activation in HEK293 cells expressing a given human TLR. Results shown reveal values minus controls and were done in triplicate. Results show that rev-peptideX2 had activity greater than peptideX2, which was in turn greater than pentide, with TLR2, and that all of the peptides tested had negligible activity with other TLRs.

FIG. 8 shows results from an in vivo study of the effects of PeptideX2 (SEQ ID NO: 1) and pentide (YLQMN, SEQ ID NO:5, “modified peptide X₂”) in collagen-induced arthritis in Lewis rats as an animal model of rheumatoid arthritis. Five Lewis rats were prepared using collagen induced arthritis protocol. At day 15, each rat was given daily dosing for next 15 days consisting of: placebo (0 ug/ml), another rat 200 μg/ml peptideX2, another rat 300 μg/ml peptideX2, another rat 500 μg/ml of modified peptideX2 (YLQMN), and another rat given positive control consisting of prednisolone 10 mg/kg/dose. At day 15, the rat receiving single IV dose to achieve 300 μg/ml concentration went into immediate respiratory distress and convulsions. Although the animal regained responsiveness to toe pinch within six minutes, the animal remained prostrate. Thirty-eight minutes after injection, the rat went into convulsions again and died. Necropsy (SOP 1075) revealed a urinary bladder full of blood, dark kidneys and liver, darkly mottled lungs with lesions on the lower right lobe. No adverse effects were observed in any other animal.

### BRIEF DESCRIPTION OF THE SEQUENCES

SEQ ID NO: 1 is the amino acid sequence KSIAYLQMNSLK of PeptideX2.

SEQ ID NO: 2 is the amino acid sequence GKSIAYLQMNSLKG of G2PeptideX2.

SEQ ID NO: 3 is the amino acid sequence GKAAYLQMNAAKG of PeptideX2-13.

SEQ ID NO: 4 is the amino acid sequence G-X₂-AAYLQMNAA-X₇-G wherein X₂ and X₇ are independently selected from K and R.

SEQ ID NO: 5 is the amino acid sequence YLQMN (also referred to as “pentide” or “peptideX2 core”)

SEQ ID NO: 6 is the spacer amino acid sequence Glu-Gly-Lys-Ser-Ser-Gly-Ser-Gly-Ser-Glu-Ser-Lys-Val-Asp.

SEQ ID NO: 7 is the spacer amino acid sequence Lys-Glu-Ser-Gly-Ser-Val-Ser-Ser-Glu-Gln-Leu-Ala-Gln-Phe-Arg-Ser-Leu-Asp.

SEQ ID NO: 8 is the spacer amino acid sequence Gly-Gly-Gly-Gly-Ser.

SEQ ID NO: 9 is the FLAG™ peptide epitope tag sequence DYKDDDDK.

SEQ ID NO: 10 is the amino acid sequence GRAAYLQMNAARG of K2R/K12R-substituted PeptideX2-13.

SEQ ID NO: 11 is the amino acid sequence X₁X₂X₃X₄YLQMNX₅X₆X₇X₈ as set forth in SEQ ID NO: 11, wherein X₁ and X₈ are each independently glycine (G) or proline (P), X₂ and X₇ are each independently lysine (K) or arginine (R), and X₃, X₄, X₅ and X₆ are each independently alanine (A) or nothing.

SEQ ID NO: 12 is the amino acid sequence of PKAAYLQMNAAKP.

SEQ ID NO: 13 is the amino acid sequence of GKAYLQMNAKG.

SEQ ID NO: 14 is the amino acid sequence of GKAAYLQMNAAKP.

SEQ ID NO: 15 is the amino acid sequence of PKAAYLQMNAAKG.

SEQ ID NO: 16 is the amino acid sequence of NMQLY (also referred to as “reverse core” or “reverse modified peptideX2”).

SEQ ID NO: 17 is the amino acid sequence of GKAANMQLYAAKG.

SEQ ID NO:18 is the amino acid sequence of LQMN (also referred to as “modified peptideX2 (4)”).

SEQ ID NO:19 is the amino acid sequence of QMN (also referred to as “modified peptideX2 (3)”).

SEQ ID NO:20 is the amino acid sequence of GKVVYLQMNVVKG.

SEQ ID NO: 21 is the amino acid sequence of GKLLYLQMNLLKG.

SEQ ID NO:22 is the amino acid sequence of GKYLQMNKG.

SEQ ID NO:23 is the amino acid sequence of GYLQMNG.

SEQ ID NO:24 is the amino acid sequence of GRSSAYLQMNASSRG.

SEQ ID NO:25 is the amino acid sequence of GRSSANMQLYASSRG.

### DETAILED DESCRIPTION

The present disclosure relates to immunomodulatory polypeptides and peptides, and in particular to an artificial tridecameric peptide referred to herein as PeptideX2-13 which comprises the amino acid sequence GKAAYLQMNAAKG [SEQ ID NO: 3] and to variants of PeptideX2-13 that
(a) comprise or consist of the amino acid sequence of general formula:

X₁X₂X₃X₄YLQMNX₅X₆X₇X₈ as set forth in SEQ ID NO: 11, wherein X₁ and X₈ are each independently glycine (G) or proline (P), X₂ and X₇ are each independently lysine (K) or arginine (R), and X₃, X₄, X₅ and X₆ are each independently alanine (A) or nothing,
(b) comprise or consist of YLQMN as set forth in SEQ ID NO: 5 other than SEQ ID NO: 1 or other related polypeptides disclosed in U.S. Pat. Nos. 9,816,989 and 10,365,275,
(c) comprise or consist of NMQLY as set forth in SEQ ID NO: 16, or
(d) comprise or consist of the amino acid sequence GKAANMQLYAAKG as set forth in SEQ ID NO: 17.

In certain embodiments, the variants of PeptideX2-13 comprise the amino acid sequence of general formula: G-X₁-AAYLQMNAA-X₂-G as set forth in SEQ ID NO: 4 wherein X₁ and X₂ are independently selected from K and R, for example, GRAAYLQMNAARG (SEQ ID NO: 10).

In certain embodiments, the variants of PeptideX2-13 comprise or consist of:
(a) GRAAYLQMNAARG as set forth in SEQ ID NO: 10,
(b) PKAAYLQMNAAKP as set forth in SEQ ID NO: 12,
(c) GKAYLQMNAKG as set forth in SEQ ID NO: 13,
(d) GKAAYLQMNAAKP as set forth in SEQ ID NO: 14,
(e) PKAAYLQMNAAKG as set forth in SEQ ID NO: 15, or
(g) GKYLQMNKG as set forth in SEQ ID NO: 22.

In certain embodiments, the variant of PeotideX2-13 consists of YLQMN as set forth in SEQ ID NO: 5 or NMQLY as set forth in SEQ ID NO: 16.

The herein disclosed artificial immunomodulatory polypeptide PeptideX2-13 (as well as its variant consisting of YLQMN as set forth in SEQ ID NO: 5) was protective in an in vivo rheumatoid arthritis (RA) animal model, and exhibited functional biological interactions with TLR2 that resembled the biological effects of the previously described immunoglobulin heavy chain-derived dodecapeptide known as PeptideX2 [SEQ ID NO: 1] as described in U.S. Pat. Nos. 9,816,989; 10,365,275; U.S. Pat. 10,871, 489; and WO 2014/165282. Embodiments of the present disclosure are based in pertinent part on the discovery that structural modifications made by introducing amino acid substitutions, deletions, and additions to the dodecameric PeptideX2 sequence KSIAYLQMNSLK [SEQ ID NO: 1] to arrive at the herein disclosed artificial tridecameric PeptideX2-13 sequence GKAAYLQMNAAKG [SEQ ID NO: 3] (as well as its variants, such as YLQMN [SEQ ID NO:5] and NMQLY [SEQ ID NO: 16]) resulted in an immunomodulatory polypeptide having unforeseeably improved properties, including increased stability in solution and decreased tendency to self-aggregate and become insoluble at physiologically relevant pH, and consequently an improved physiological safety profile as evidenced by the observation in an animal model that PeptideX2-13 was tolerated and not toxic at a certain higher concentrations than was the case for previously described PeptideX2. Like the previously described PeptideX2 [SEQ ID NO: 1], the presently described PeptideX2-13 [SEQ ID NO: 3] (as well as some of its variants, such as YLQMN [SEQ ID NO:5] and NMQLY [SEQ ID NO: 16]) may be used to elicit the expression of cytokines, including in particular interleukin-6 (IL-6) and tumor necrosis factor-alpha (TNF-α) and also interleukin-10 (IL-10), by peripheral blood leukocytes (PBL). This property is quite remarkable in that this cytokine expression pattern by PBL is not characteristic of either a classic helper T cell type 1 (Th1) or a helper T cell type 2 (Th2) response. Furthermore, PeptideX2-13 (as well as some of its variants, such as YLQMN [SEQ ID NO:5] and NMQLY [SEQ ID NO:16]), like PeptideX2, may preferentially bind to and thus identify a human neutrophil subpopulation. As described herein for the first time and presented in greater detail below, PeptideX2-13 (as well as some of its variants, such as YLQMN [SEQ ID NO:5] and NMQLY [SEQ ID NO:16]) is shown here to be capable of delivering a specific transcriptional activation signal to innate immune system cells via preferential binding interactions with human TLR2 at discrete and detectable signaling levels that were nevertheless well below the levels delivered by natural PAMP ligands for this TLR.

By these effects on local and/or systemic immunologic status (e.g., hyperinflammatory vs. hypoinflammatory, altered cytokine profile, etc.), which may vary as a function of PeptideX2-13 (or a variant thereof) dosage parameters (e.g., concentration, timing, absence or presence of competing TLR2/4 ligands such as PAMPs or DAMPs, activation status of target cells, host clinical status, valency, etc.), the herein described PeptideX2-13 polypeptide or variants thereof are thus believed according to non-limiting theory to provide unprecedentedly useful immunomodulatory properties. The presently disclosed PeptideX2-13 and its variants afford such properties through their ability to alter (e.g., increase or decrease in a statistically significant manner) the activity levels of one or more cellular regulators of immune status, such as biological signals that are transduced through TLR2 and/or TLR4.

According to certain preferred embodiments and further according to non-limiting theory, beneficial uses of the presently disclosed PeptideX2-13 and variants thereof relate to unexpected advantages that are obtained by their dual functioning (i) as weak agonists of TLR2 and/or TLR4, through which the biological signals that are delivered by PeptideX2-13 are transduced but are qualitatively and quantitatively less profound than TLR2/TLR4 activation signals that are transduced in response to previously described PAMPs, DAMPS or other TLR ligands, and (ii) as antagonists of PAMPs and/or DAMPs by virtue of their competitive, albeit low affinity, binding to TLR2 and TLR4. In this respect, the presently disclosed immunomodulatory polypeptides (e.g., PeptideX2-13 and variants thereof) surprisingly permit the innate immune system to mediate a moderate inflammatory response instead of the exuberant hyperinflammatory reaction that characterizes early sepsis, without driving the immune system to the immunosuppressed state that otherwise often subsequently predominates in later stages of sepsis. Similarly, the presently disclosed immunomodulatory polypeptides surprisingly conferred a moderating effect on the deleterious autoimmune inflammatory response in an art-accepted animal model of rheumatoid arthritis (RA).

Thus, according to certain embodiments disclosed herein there is provided non-self-aggregating, solution stable immunomodulatory polypeptides (e.g., PeptideX2-13 and certain variants thereof, such as YLQMN [SEQ ID NO:5] and NMQLY [SEQ ID NO:16]) capable of weakly activating a human peripheral white blood cell subset (believed according to non-limiting theory to comprise a neutrophil subset) via TLR2 and/or TLR4 in a manner that rapidly alters the inflammatory cytokine expression profile of such innate immune cells. PeptideX2-13 (as well as certain variants thereof) surprisingly mediates these effects without compromising early-phase antimicrobial activity of these cells and without activating or inducing the immunosuppressive cytokine profile that may otherwise be elaborated by phagocytes (macrophages and dendritic cells) involved in the clearance of peripheral white blood cells including neutrophils that have undergone apoptosis or other DAMP.

Accordingly, without wishing to be bound by theory, it is believed that the dual weak TLR agonist/potent PAMP/DAMP antagonist activity of immunomodulatory polypeptides disclosed herein (e.g., PeptideX2-13 and certain variants thereof, such as YLQMN [SEQ ID NO: 5] and NMQLY [SEQ ID NO:16]) advantageously avoid permissive conditions for an overwhelming microbial (e.g., bacterial) infection in sepsis, at least in part by interfering with the onset of immunosuppressive conditions that would otherwise preclude the adaptive immune system from mounting a vigorous anti-microbial response against the pathogen. The presently disclosed immunomodulatory polypeptides feature the advantageous and previously unpredicted properties of (i) binding recognition of, and beneficially weak signal transduction activity via, human TLR2 and/or TLR4; (ii) modulation of TLR2 and/or TLR4 activation by down-regulating the innate immune response proinflammatory component when present at low concentrations; and (iii) ease of manufacture of an active moiety that may be as small as a tridecapeptide in the case of PeptideX2-13 (SEQ ID NO: 3) and K2R/K12R-substituted PeptideX2-13 (SEQ ID NO: 10) and as small as a pentapeptide for the herein described YLQMN (SEQ ID NO: 5) core peptide sometimes referred to herein as “pentide” and in which TLR2/4-interacting biological activity is believed to reside. The present immunomodulatory polypeptides are expected to be non-immunogenic in humans, avoiding the challenges of monoclonal antibody humanization, CDR optimization and production.

In certain embodiments, the artificial immunomodulatory polypeptides disclosed herein are anti-inflammatory when soluble, but become pro-inflammatory when insoluble. Without wishing to be bound by theory, it is hypothesized that both the anti-inflammatory and pro-inflammatory activities of such immunomodulatory polypeptides may be due to weak binding of the polypeptides to TLR2>TLR4. Preferably, the immunomodulatory polypeptides provided herein are more soluble in a pharmaceutically acceptable solution (e.g., 0.2% acetic acid, 0.375 M Hepes, 0.375% glycerol, pH 7; 0.08% acetic acid, 0.4 M Hepes, 0.4% glycerol, pH6; or 0.08% acetic acid, 0.08 M Hepes, 17.6% glycerol, pH7) than peptideX2, are able to be prepared at higher concentrations than peptideX2 (e.g., at least 2 mg/ml, at least 3 mg/ml, at least 4 mg/ml, or at least 5 mg/ml in a pharmaceutically acceptable solution), may be administrated at higher amounts or concentrations than peptideX2 (e.g., at 200 to 600 μg/ml such as 200 to 500 μg/ml, 200 to 400 μg/ml or 200 to 300 μg/ml), and/or have higher anti-inflammatory activity than peptideX2.

Also without wishing to be bound by theory, it is hypothesized that the herein described artificial tridecapeptide PeptideX2-13 [e.g., SEQ ID NO: 3] and variants thereof (e.g., YLQMN [SEQ ID NO:5] and NMQLY [SEQ ID NO: 16]) may also be usefully exploited in a number of medically relevant contexts, including improving outcomes in organ transplantation by decreasing (e.g., in a statistically significant manner relative to appropriate control conditions) immunological rejection mechanisms that may otherwise be manifest in host responses to organ or tissue grafts, including allografts and xenografts.

The distinctive immunomodulatory properties of PeptideX2-13 and variants thereof (e.g., YLQMN [SEQ ID NO:5] and NMQLY [SEQ ID NO: 16]) may also find beneficial uses in treating cancer, including promotion of mechanisms of tumor cell killing and/or in inhibition of metastasis, and also in treating graft-versus host disease (GVHD) in bone marrow transplant recipients, and in the context of reproductive medicine, for example, to improve fertility (including pregnancy initiated following in vitro fertilization (IVF)) by promoting embryonic implantation, and in reducing the severity of symptoms associated with preeclampsia and eclampsia during pregnancy. Immunomodulatory polypeptides comprising or consisting of the herein described PeptideX2-13 (SEQ ID NO: 3) or variants thereof (e.g., YLQMN [SEQ ID NO: 5] and NMQLY [SEQ ID NO:16]) may additionally be advantageously administered in treating inflammatory conditions such as atherosclerosis and uveitis, and autoimmune diseases such as rheumatoid arthritis, psoriatic arthritis, Crohn's disease, systemic lupus erythematosus and other autoimmune diseases, and may more generally induce a heretofore unprecedented non-Th1, non-Th2 immunological response that is characterized by up-regulated (e.g., increased in a statistically significant manner relative to an appropriate control) release of at least one of (and in certain preferred embodiments at least two of) IL-6, IL-10 and TNFα, by peripheral white blood cells.

According to a certain embodiment disclosed herein, there is provided PeptideX2-13 (SEQ ID NO: 3), an immunomodulatory polypeptide that recognizes and specifically binds to a cell surface structure that is present on certain human and murine neutrophils, and for which a cognate cell surface binding structure resides in the toll-like receptors TLR2 and TLR4. The immunomodulatory polypeptides described herein, for instance, polypeptides comprising the artificial tridecameric PeptideX2-13 [SEQ ID NO: 3] or its variants may thus find immunotherapeutic uses.

For example, in certain embodiments, the presently disclosed PeptideX2-13 and its variants (e.g., YLQMN [SEQ ID NO:5] and NMQLY [SEQ ID NO: 16]) may be beneficially exploited to treat, or to reduce (e.g., decrease with statistical significance) the severity or likelihood of occurrence of, sepsis in a subject such as a human patient or other mammal. Accordingly and without intending to be bound by theory, these immunomodulatory polypeptides may thus be useful as agents that alter (e.g., increase or decrease in a statistically significant manner, and in certain preferred embodiments, decrease) PAMP ligand binding to neutrophil TLR2 and/or TLR4, by binding to TLR2 and/or TLR4 without eliciting the neutrophil activation that would otherwise characterize a full-fledged TLR-ligand response.

In these and other related embodiments, the presently disclosed immunomodulatory polypeptides may find uses as agents for regulated immunosuppressive treatment. In certain contemplated embodiments, PeptideX2-13 and its variants (e.g., YLQMN [SEQ ID NO:5] and NMQLY [SEQ ID NO: 16]) may suppress immune effector cell proliferation in response to an allograft or xenograft, without severely compromising host immune potential and without inducing massive inflammation. The herein disclosed immunomodulatory polypeptides may, for example by way of illustration and not limitation, be used in transplant patients to prevent or ameliorate graft rejection and/or GVHD.

In certain other embodiments, the present disclosure relates to the use of the herein disclosed immunomodulatory polypeptides (e.g., polypeptides comprising or consisting of SEQ ID NO: 3, 4, 5, 10, 11, 12, 13, 14, 15, 16, 17, or 22) in fertility treatments, and particularly for use in increasing the likelihood of embryonic implantation in a variety of contexts, including natural pregnancies as well as those that may result from introduction into a surrogate mother (such as a pseudopregnant female) of an embryo produced by IVF. In other embodiments, the herein disclosed immunomodulatory polypeptides (e.g., polypeptides comprising or consisting of SEQ ID NO: 3, 4, 5, 10, 11, 12, 13, 14, 15, 16, 17, or 22) may be used in the prevention and/or treatment of pregnancy related conditions including preeclampsia and eclampsia. In such applications of the herein described compositions and methods, the present immunomodulatory polypeptide may be contacted with the embryo in vitro or in vivo and/or with the pregnant or pseudopregnant female in whom it is desired to promote fertility.

Other herein contemplated embodiments encompass the use of the disclosed immunomodulatory polypeptides (e.g., polypeptides comprising or consisting of SEQ ID NO: 3, 4, 5, 10, 11, 12, 13, 14, 15, 16, 17 or 22) or the use of isolated specific polyclonal antibodies and/or engineered antibodies specifically targeted to bind to such YLQMN [SEQ ID NO: 5] or NMQLY [SEQ ID NO: 16]-containing polypeptides to control immunity and inflammation, for example, as controlled immunosuppressants in the treatment of autoimmune diseases and disorders as provided herein, such as rheumatoid arthritis, psoriatic arthritis, Crohn's disease, and others.

It will be appreciated that in certain herein disclosed embodiments, an isolated immunomodulatory polypeptide comprising or consisting of the PeptideX2-13 sequence set forth in SEQ ID NO:3 or its variants as provided herein may comprise 31, 30, 29, 28, 27, 26, 25, 24, 23, 22, 21, 20, 19, 18, 17, 16, 15, 14, or 13, optionally 12, 11, 10, 9, 8, 7, 6, or 5 amino acids, in certain embodiments with SEQ ID NO: 3, 10, 12, 13, 14, 15, 17, or 22 or SEQ ID NO: 4 or 11 being situated at the N-terminus with 0-19 amino acids of any sequence forming the C-terminus, or SEQ ID NO: 3, 10, 12, 13, 14, 15, 17, or 22 or SEQ ID NO: 4 or 11 may be situated at the C-terminus with 0-19 amino acids of any sequence forming the N-terminus, or SEQ ID NO: 3, 10, 12, 13, 14, 15, 17 or 22 or SEQ ID NO: 4 or 11 may be situated at neither the N-terminus nor the C-terminus and may be linked via peptide bonds to additional amino acids of any sequence that form N- and C-termini, so long as the entire polypeptide is of no more than 31, 30, 29, 28, 27, 26, 25, 24, 23, 22, 21, 20, 19, 18, 17, 16, 15, 14, or 13, optionally 12, 11, 10, 9, 8, 7, 6, or 5 amino acids in length.

Depressed levels of overall endometrial/decidual IgG levels have previously been correlated with advancing trimesters during the course of pregnancy (Kutteh et al., 2001 Am J Obstet. Gynecol. 184:865) but no identification of individual IgG species or their specificities or biological effects have been described. Prior reports of an immunoglobulin component that was present in immunosuppressive endometrial extracts traced the immunosuppressive activity to the immunoglobulin constant (Fc) domain (Haruyama et al., 1991 J. Reprod. Immunol. 19:1; Kitano et al, 1990 Acta Obst. Gynaec. Jpn. 42 (7): 739), which does not mediate specific antigen recognition and binding. By pointing to a non-antigen specific biological effect mediated by the immunoglobulin constant (C) region, these earlier reports teach away from the presently described PeptideX2-13 and its variants, which comprise artificial amino acid sequence variants engineered by modifying short regions of immunomodulatory polypeptide regions of a distinctive immunoglobulin heavy chain variable (V) region sequence, which is found in an IgG immunoglobulin termed “IgX”, as described in, e.g., U.S. Pat. Nos. 9,816,989; 10,365,275; U.S. Pat. 10,871, 489; and WO 2014/165282.

### Polypeptides and Proteins

The terms “polypeptide” “protein” and “peptide” and “glycoprotein” are used interchangeably and mean a polymer of amino acids not limited to any particular length. The term does not exclude modifications such as myristylation, sulfation, glycosylation, phosphorylation and addition or deletion of signal sequences. The terms “polypeptide” or “protein” means one or more chains of amino acids, wherein each chain comprises amino acids covalently linked by peptide bonds, and wherein said polypeptide or protein can comprise a plurality of chains non-covalently and/or covalently linked together by peptide bonds, having the sequence of native proteins, that is, proteins produced by naturally-occurring and specifically non-recombinant cells, or genetically-engineered or recombinant cells, and comprise molecules having the amino acid sequence of the native protein, or molecules having deletions from, additions to, and/or substitutions of one or more amino acids of the native sequence. Thus, a “polypeptide” or a “protein” can comprise one (termed “a monomer”) or a plurality (termed “a multimer”) of amino acid chains. The terms “peptide,” “polypeptide” and “protein” specifically encompass the immunomodulatory polypeptides of the present disclosure, or sequences that have deletions from, additions to, and/or substitutions of one or more amino acid of an immunomodulatory polypeptide.

The terms “isolated protein” and “isolated polypeptide” referred to herein means that a subject protein or polypeptide (1) is free of at least some other proteins or polypeptides with which it would typically be found in nature, (2) is essentially free of other proteins or polypeptides from the same source, e.g., from the same species, (3) is expressed by a cell from a different species, (4) has been separated from at least about 50 percent of polynucleotides, lipids, carbohydrates, or other materials with which it is associated in nature, (5) is not associated (by covalent or noncovalent interaction) with portions of a protein or polypeptide with which the “isolated protein” or “isolated polypeptide” may be associated in nature, (6) is operably associated (by covalent or noncovalent interaction) with a polypeptide with which it is not associated in nature, or (7) does not occur in nature. Such an isolated protein or polypeptide can be encoded by genomic DNA, cDNA, RNA or other RNA, of may be of synthetic origin according to any of a number of well-known chemistries for artificial peptide and protein synthesis, or any combination thereof. In certain embodiments, the isolated protein or polypeptide is substantially free from proteins or polypeptides or other contaminants that are found in its natural environment that would interfere with its use (therapeutic, diagnostic, prophylactic, research or otherwise).

The term “polypeptide fragment” refers to a polypeptide, which can be monomeric or multimeric, that has an amino-terminal deletion, a carboxyl-terminal deletion, and/or an internal deletion or substitution of a naturally occurring or recombinantly produced polypeptide. As used herein, “contiguous amino acids” refers to covalently linked amino acids corresponding to an uninterrupted linear portion of a disclosed amino acid sequence. In certain embodiments, a polypeptide fragment can comprise an amino acid chain at least 5 to about 500 amino acids long. It will be appreciated that in certain embodiments, fragments are at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 150, 200, 250, 300, 350, 400, or 450 amino acids long. In certain embodiments, fragments are at most 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 150, 200, 250, 300, 350, 400, or 450 amino acids long. In certain embodiments, fragments are 5 to 31 amino acids in length, such as 5 to 30, 5 to 25, 5 to 20, 5 to 15, and 5 to 13 amino acids in length.

The immunomodulatory polypeptides disclosed herein are preferably not naturally occurring. In certain embodiments, the amino acid sequences of such polypeptides are not present in any natural occurring proteins. In certain embodiments, the immunomodulatory polypeptides contain amino acid modifications that do not occur in nature.

In certain embodiments, immunomodulatory polypeptides disclosed herein are 5 to 31 amino acids in length. In certain embodiments, immunomodulatory polypeptides disclosed herein 5 to 30 amino acids in length. In certain embodiments, immunomodulatory polypeptides disclosed herein 5 to 25 amino acids in length. In certain embodiments, immunomodulatory polypeptides disclosed herein 5 to 20 amino acids in length. In certain embodiments, immunomodulatory polypeptides disclosed herein 5 to 15 amino acids in length. In certain embodiments, immunomodulatory polypeptides disclosed herein 5 to 13 amino acids in length.

The term “protein variant,” “variant of a protein,” “polypeptide variant,” or “variant of a polypeptide” refers to a protein or polypeptide (“a second protein or polypeptide”) that has one or more amino acid insertions, deletions, and/or substitutions compared to another protein or polypeptide (“a first protein or polypeptide” or “a parent protein or polypeptide”). In this case, the second protein or polypeptide may be referred to as a variant of the first protein or polypeptide. Typically, a protein or polypeptide variant retains one or more structural (e.g., a core sequence) and/or functional (e.g., an immunomodulatory activity) features of its parent protein or polypeptide.

A “variant of PeptideX2-13” as used herein refers to a polypeptide comprising or consisting of the core sequence YLQMN (SEQ ID NO: 5) or the reverse core sequence NMQLY (SEQ ID NO:16). Preferably, a variant of PeptideX2-13 has one or more of the following features: (1) an immunomodulatory activity, especially an anti-inflammatory activity, such as shown in in vivo animal model for an immunological disorder, (2) capability of interacting with TLR2 and/or TLR4, such as shown by in vitro TLR2 or TLR4-driven NF-κB activation in HEK293 cell lines transfected with human TLR2 or TLR4, (3) solubility similar to or higher than that of PeptideX2 (SEQ ID NO: 1) in an aqueous buffer (e.g., a DPBS or Hepes buffer), and/or (4) less toxic than PeptideX2 when administered to a subject.

In certain embodiments, the variants of PeptideX2-13 disclosed herein comprise the amino acid sequence of general formula: X₁X₂X₃X₄YLQMNX₅X₆X₇X₈ as set forth in SEQ ID NO: 11, wherein X₁ and X₈ are each independently glycine (G) or proline (P), X₂ and X₇ are each independently lysine (K) or arginine (R), and X₃, X₄, X₅ and X₆ are each independently alanine (A) or nothing. Exemplary variants of PeptideX2-13 comprise the amino acid sequence of general formula: G-X₁-AAYLQMNAA-X₂-G as set forth in SEQ ID NO: 4 wherein X₁ and X₂ are independently selected from K and R, for example, GRAAYLQMNAARG (SEQ ID NO: 10). Additional exemplary variants of PeptideX2-13 comprise:
(a) GRAAYLQMNAARG as set forth in SEQ ID NO: 10,
(b) PKAAYLQMNAAKP as set forth in SEQ ID NO: 12,
(c) GKAYLQMNAKG as set forth in SEQ ID NO: 13,
(d) GKAAYLQMNAAKP as set forth in SEQ ID NO: 14,
(e) PKAAYLQMNAAKG as set forth in SEQ ID NO: 15, or
(g) GKYLQMNKG as set forth in SEQ ID NO: 22.

In certain embodiments, the variants of PeptideX2-13 comprise or consist of YLQMN as set forth in SEQ ID NO: 5 other than SEQ ID NO: 1 or other related polypeptides disclosed in U.S. Pat. Nos. 9,816,989 and 10,365,275, or comprise or consist of NMQLY as set forth in SEQ ID NO: 16. Exemplary variants of PeptideX2-13 consists of YLQMN as set forth in SEQ ID NO: 5 or NMQLY as set forth in SEQ ID NO: 16. An additional exemplary variant of PeptideX2-13 comprise the amino acid sequence GKAANMQLYAAKG as set forth in SEQ ID NO: 17.

Polypeptides may comprise a signal (or leader) sequence at the N-terminal end of the protein, which co-translationally or post-translationally directs transfer of the protein. The polypeptide may also be fused in-frame or conjugated to a linker or other sequence for ease of synthesis, purification or identification of the polypeptide (e.g., poly-His), or to enhance binding of the polypeptide to a solid support. Fusion domain polypeptides may be joined to the polypeptide at the N-terminus and/or at the C-terminus, and may include as non-limiting examples, immunoglobulin-derived sequences such as Ig constant region sequences or portions thereof, affinity tags such as His tag (e.g., hexahistidine or other polyhistidine), FLAG™ (DYKDDDDK, SEQ ID NO: 9) or myc or other peptide affinity tags, detectable polypeptide moieties such as green fluorescent protein (GFP) or variants thereof (e.g., yellow fluorescent protein (YFP), blue fluorescent protein (BFP), other aequorins or derivatives thereof, etc.) or other detectable polypeptide fusion domains, enzymes or portions thereof such as glutathione-S-transferase (GST) or other known enzymatic detection and/or reporter fusion domains, and the like, as will be familiar to the skilled artisan.

Cysteine-containing peptides may be used as fusion peptides that can be joined to the N-and/or C-terminus of the herein described peptideX2-13 (e.g., SEQ ID NO: 3) or its variants to permit ready assembly of such polypeptides into disulfide-crosslinked dimers, trimers, tetramers or higher multimers according to established methodologies. For example, fusion polypeptides containing immunoglobulin gene superfamily member-derived sequences that include cysteine residues capable of forming interchain disulfide bridges are well known, as also are other strategies for engineering S-S linked multimers (e.g., Reiter et al., 1994 Prot. Eng. 7:697; Zhu et al., 1997 Prot. Sci. 6:781; Mabry et al., 2010 Mabs 2:20; Gao et al., 1999 Proc. Nat. Acad. Sci. USA 96:6025; Lim et al., 2010 Biotechnol. Bioeng. 106:27). Alternative approaches are also contemplated for grafting peptide sequences that promote multimer assembly as fusion domains onto a desired polypeptide such as the herein described immunomodulatory peptides (e.g., Fan et al., 2008 FASEB J. 22:3795).

Polypeptide modifications may be effected biosynthetically and/or chemically according to a wide variety of well-known methodologies, and may also include conjugation to carrier proteins (e.g., keyhole limpet hemocyanin (KLH), bovine serum albumin (BSA), ovalbumin (OVA) or other molecules), and covalent or non-covalent immobilization on solid supports. Chemical or biosynthetic conjugation to a carrier is contemplated, according to certain embodiments, for generation of conjugates that are multivalent with respect to the herein described peptideX2-13 (e.g., a polypeptide that contains SEQ ID NO: 3) or its variants. For example and according to non-limiting theory, it is believed that target cells for immunomodulation (e.g., a neutrophil subset) that express certain cell surface receptors for peptideX2-13 may transduce biological signals more efficiently when stimulated by a multivalent peptideX2-13 structure, relative to the level of induction that may be afforded by a monovalent peptideX2-13 structure. Also contemplated is detectable labeling with detectable indicator moieties (sometimes referred to as reporter moieties) such as fluorophores (e.g., FITC, TRITC, Texas Red, etc.). Examples of a broad range of detectable indicators (including colorimetric indicators) that may be selected for specific purposes are described in Haugland, 2002 Handbook of Fluorescent Probes and Research Products-Ninth Ed., Molecular Probes, Eugene, OR; in Mohr, 1999 J. Mater. Chem., 9:2259-2264; in Suslick et al., 2004 Tetrahedron 60:11133-11138; and in U.S. Pat. No. 6,323,039. (See also, e.g., Fluka Laboratory Products Catalog, 2001 Fluka, Milwaukee, WI; and Sigma Life Sciences Research Catalog, 2000, Sigma, St. Louis, MO.) A detectable indicator may be a fluorescent indicator, a luminescent indicator, a phosphorescent indicator, a radiometric indicator, a dye, an enzyme, a substrate of an enzyme, an energy transfer molecule, or an affinity label.

Other detectable indicators for use in certain embodiments contemplated herein include affinity reagents such as antibodies, lectins, immunoglobulin Fc receptor proteins (e.g., Staphylococcus aureus protein A, protein G or other Fc receptors), avidin, biotin, other ligands, receptors or counterreceptors or their analogues or mimetics, and the like. For such affinity methodologies, reagents for immunometric measurements, such as suitably labeled antibodies or lectins, may be prepared including, for example, those labeled with radionuclides, with fluorophores, with affinity tags, with biotin or biotin mimetic sequences or those prepared as antibody-enzyme conjugates (see, e.g., Weir, D. M., Handbook of Experimental Immunology, 1986, Blackwell Scientific, Boston; Scouten, W. H., 1987 Methods in Enzymology 135:30-65; Harlow and Lane, Antibodies: A Laboratory Manual, 1988 Cold Spring Harbor Laboratory, Cold Spring Harbor, NY; Haugland, Handbook of Fluorescent Probes and Research Products-Ninth Ed., 2002 Molecular Probes, Eugene, OR; Scopes, R. K., Protein Purification: Principles and Practice, 1987, Springer-Verlag, NY; Hermanson, G. T. et al., Immobilized Affinity Ligand Techniques, 1992, Academic Press, Inc., NY; Luo et al., 1998 J. Biotechnol. 65:225 and references cited therein).

Amino acid sequence modification(s) of the immunomodulatory polypeptides (e.g., PeptideX2-13 [SEQ ID NO: 3] sequence-containing polypeptide) described herein are contemplated. For example, it may be desirable to improve the binding affinity and/or other biological properties of the immunomodulatory polypeptide. For instance, amino acid sequence variants may be prepared by introducing appropriate nucleotide changes into a polynucleotide that encodes the immunomodulatory polypeptide or by peptide synthesis. Such modifications include, for example, deletions from, and/or insertions into and/or substitutions of, residues within the amino acid sequence of the immunomodulatory polypeptide. Any combination of deletion, insertion, and substitution may be made to arrive at the final immunomodulatory polypeptide variant, provided that the final construct possesses the desired characteristics (e.g., suppresses cell proliferation in a mixed lymphocyte reaction, or induces elaboration by peripheral blood white cells of the non-Th1/non-Th2 cytokine profile as described herein, or exhibits activity in a preimplantation factor activity assay such as those described in U.S. Pat. Nos. 5,646,003, 5,981,198, or WO 2005/040196). The amino acid changes also may alter post-translational processes of the immunomodulatory polypeptide, such as changing the number or position of glycosylation sites.

Determination of the three-dimensional structures of representative polypeptides (e.g., PeptideX2-13 [SEQ ID NO: 3] or its variants [SEQ ID NOS: 4, 5, 10, 11, 12, 13, 14, 15, 16, 17 and 22] or polypeptides comprising PeptideX2-13 or its variants may be made through routine methodologies such that substitution, addition, deletion or insertion of one or more amino acids with selected natural or non-natural amino acids can be virtually modeled for purposes of determining whether a so derived structural variant retains the space-filling properties of presently disclosed species. See, for instance, Donate et al., 1994 Prot. Sci. 3:2378; Bradley et al., Science 309:1868-1871 (2005); Schueler-Furman et al., Science 310:638 (2005); Dietz et al., Proc. Nat. Acad. Sci. USA 103:1244 (2006); Dodson et al., Nature 450:176 (2007); Qian et al., Nature 450:259 (2007); Raman et al. Science 327:1014-1018 (2010). Some additional non-limiting examples of computer algorithms that may be used for these and related embodiments, such as for rational design of immunomodulatory polypeptides as provided herein, include VMD which is a molecular visualization program for displaying, animating, and analyzing large biomolecular systems using 3-D graphics and built-in scripting (see the website for the Theoretical and Computational Biophysics Group, University of Illinois at Urbana-Champagne, at ks.uiuc.edu/Research/vmd/).

Many other computer programs are known in the art and available to the skilled person and which allow for determining atomic dimensions from space-filling models (van der Waals radii) of energy-minimized conformations; GRID, which seeks to determine regions of high affinity for different chemical groups, thereby enhancing binding, Monte Carlo searches, which calculate mathematical alignment, and CHARMM (Brooks et al. (1983) J. Comput. Chem. 4:187-217) and AMBER (Weiner et al (1981) J. Comput. Chem. 106:765), which assess force field calculations, and analysis (see also, Eisenfield et al. (1991) Am. J. Physiol. 261: C376-386; Lybrand (1991) J. Pharm. Belg. 46:49-54; Froimowitz (1990) Biotechniques 8:640-644; Burbam et al. (1990) Proteins 7:99-111; Pedersen (1985) Environ. Health Perspect. 61:185-190; and Kini et al. (1991) J. Biomol. Struct. Dyn. 9:475-488). A variety of appropriate computational computer programs are also commercially available, such as from Schrödinger (Munich, Germany).

### Antibodies

Certain embodiments of the present disclosure include antibodies that specifically bind to a polypeptide containing the artificial PeptideX2-13 amino acid sequence [SEQ ID NO: 3] or the amino acid sequence set forth in SEQ ID NO: 4, 5, 10, 11, 12 13, 14, 15, 16, 17, or 22, while certain other embodiments include antibodies that themselves include the PeptideX2-13 sequence set forth in SEQ ID NO: 3 or the amino acid sequence set forth in SEQ ID NO: 4, 5, 10, 11, 12 13, 14, 15, 16, 17, or 22. As described herein, PeptideX2-13 (SEQ ID NO: 3) and variants thereof were designed as artificial variants of PeptideX2 (SEQ ID NO: 1) which, as described in U.S. Pat. Nos. 10,365,275; 9,816,989; and WO 2014/165282, was identified as a region of a placentally derived antibody termed IgX. As also described herein, PeptideX2-13 and PeptideX2 share with IgX certain advantageous and unexpected immunomodulatory properties.

The term “antibody” (Ab) as used herein includes monoclonal antibodies, polyclonal antibodies, multispecific antibodies (e.g., bispecific antibodies), and antibody fragments so long as they exhibit the desired biological activity, e.g., specifically bind to neutrophils. The term “immunoglobulin” (Ig) is used interchangeably with “antibody” herein.

The basic antibody unit is a heterotetrameric glycoprotein composed of two identical light (L) chains and two identical heavy (H) chains. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus a variable domain (V_{H}) followed by three constant domains (C_{H}) for each of the α and γ chains and four CH domains for μ and ε isotypes. Each L chain has at the N-terminus, a variable domain (V_{L}) followed by a constant domain (C_{L}) at its other end. The V_{L} is aligned with the V_{H} and the C_{L} is aligned with the first constant domain of the heavy chain (C_{H}1). Particular amino acid residues are believed to form an interface between the light chain and heavy chain variable domains. The pairing of a V_{H} and V_{L} together forms a single antigen-binding site.

The L chain from any vertebrate species can be assigned to one of two clearly distinct types, called kappa (κ) and lambda (λ), based on the amino acid sequences of their constant domains (CL). Depending on the amino acid sequence of the constant domain of their heavy chains (CH), immunoglobulins can be assigned to different classes or isotypes. There are five classes of immunoglobulins: IgA, IgD, IgE, IgG, and IgM, having heavy chains designated alpha (α), delta (δ), epsilon (ε), gamma (γ) and mu (μ), respectively. The y and a classes are further divided into subclasses on the basis of relatively minor differences in C_{H} sequence and function, e.g., humans express the following subclasses: IgG1, IgG2, IgG3, IgG4, IgA1, and IgA2. It will be appreciated that mammals encoding multiple Ig isotypes will be able to undergo isotype class switching.

An IgM antibody consists of 5 of the basic heterotetramer units along with an additional polypeptide called J chain, and therefore contains 10 antigen binding sites, while secreted IgA antibodies can polymerize to form polyvalent assemblages comprising 2-5 of the basic 4-chain units along with J chain. In the case of IgG, the 4-chain unit is generally about 150,000 daltons. For the structure and properties of the different classes of antibodies, see, e.g., Basic and Clinical Immunology, 8th edition, Daniel P. Stites, Abba I. Terr and Tristram G. Parslow (eds.), Appleton & Lange, Norwalk, Conn., 1994, page 71, and Chapter 6.

The V domain mediates antigen binding and defines specificity of a particular antibody for its particular antigen. The gene sequence encoding the V_{H} domain has multiple copies of variable (V), diversity (D), and joining (J) segments. The gene sequence encoding the V_{L} domain contains multiple copies of V and J segments. The V_{H} and V_{L} regions undergo gene rearrangement (i.e., somatic recombination) to develop diverse antigen specificity in antibodies. The term “variable” refers to the fact that certain segments of the V domains differ extensively in sequence among antibodies.

However, the variability is not evenly distributed across the 110-amino acid span of the variable domains. Instead, the V regions consist of relatively invariant stretches called framework regions (FRs) of 15-30 amino acids separated by short regions of extreme variability called “hypervariable regions.” These hypervariable regions are the result of somatic hypermutation during the affinity maturation process, and they are typically each 9-18 amino acids long. However, they have been found to range from 4-28 amino acids in length depending upon the particular epitope. For example, CDR3 regions up to at least 22 or 23 amino acids in length have been described. See, e.g., Morea V, et al., J Mol Biol. 275 (2): 269-94 (1998) and Kabat, E. A., et al., Sequences of Proteins of Immunological Interest, Fifth Edition. NIH

### Publication No. 91-3242 (1991).

The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC).

The term “hypervariable region” when used herein refers to the amino acid residues of an antibody that are responsible for antigen binding. The hypervariable region generally comprises amino acid residues from a “complementarity determining region” or “CDR” (e.g., around about residues 24-34 (L1), 50-56 (L2) and 89-97 (L3) in the V_{L}, and around about 28-36 (H1), 50-65 (H2) and 95-102 (H3) in the V_{H}; Kabat et al., Sequences of Proteins of Immunological Interest, 5th Ed. Public Health Service, National Institutes of Health, Bethesda, Md. (1991)) and/or those residues from a “hypervariable loop” (e.g., residues 26-32 (L1), 50-52 (L2) and 91-96 (L3) in the V_{L}, and 26-32 (H1), 53-55 (H2) and 96-101 (H3) in the V_{H}; Chothia and Lesk, J. Mol. Biol. 196:901-917 (1987)).

An “isolated antibody” is one that has been separated and/or recovered from a component of its natural environment. Contaminant components of its natural environment are materials that would interfere with diagnostic or therapeutic uses for the antibody, and may include enzymes, hormones, and other proteinaceous or nonproteinaceous solutes. In preferred embodiments, the antibody is purified: (1) to greater than 95% by weight of antibody as determined by the Bradford method, and most preferably more than 99% by weight; (2) to a degree sufficient to obtain at least 15 residues of N-terminal or internal amino acid sequence by use of a spinning cup sequenator; or (3) to homogeneity by SDS-PAGE under reducing or non-reducing conditions using Coomassie blue or, silver stain. Isolated antibody includes the antibody in situ within recombinant cells since at least one component of the antibody's natural environment will not be present. Ordinarily, however, isolated antibody will be prepared by at least one purification step.

An “intact” antibody is one that comprises an antigen-binding site as well as a CL and at least heavy chain constant domains, CH 1, CH 2 and CH 3. The constant domains may be native sequence constant domains (e.g., human native sequence constant domains) or amino acid sequence variants thereof. Preferably, the intact antibody has one or more effector functions.

An “antibody fragment” is a polypeptide comprising or consisting of a portion of an intact antibody, preferably the antigen binding or variable region of the intact antibody. Examples of antibody fragments include Fab, Fab′, F(ab′)₂, and Fv fragments; diabodies; linear antibodies (see U.S. Pat. No. 5,641,870; Zapata et al., Protein Eng. 8 (10): 1057-1062 [1995]); single-chain antibody molecules; and multispecific antibodies formed from antibody fragments.

Papain digestion of antibodies produces two identical antigen-binding fragments, called “Fab” fragments, and a residual “Fc” fragment, a designation reflecting the ability to crystallize readily. The Fab fragment consists of an entire L chain along with the variable region domain of the H chain (V_{H}), and the first constant domain of one heavy chain (CH 1). Each Fab fragment is monovalent with respect to antigen binding, i.e., it has a single antigen-binding site. Pepsin treatment of an antibody yields a single large F(ab′) 2 fragment that roughly corresponds to two disulfide linked Fab fragments having divalent antigen-binding activity and is still capable of cross-linking antigen. Both the Fab and F(ab′) 2 are examples of “antigen-binding fragments.” Fab′ fragments differ from Fab fragments by having additional few residues at the carboxy terminus of the CH1 domain including one or more cysteines from the antibody hinge region. Fab′-SH is the designation herein for Fab′ in which the cysteine residue(s) of the constant domains bear a free thiol group. F(ab′) 2 antibody fragments originally were produced as pairs of Fab′ fragments that have hinge cysteines between them. Other chemical couplings of antibody fragments are also known.

The “Fc” fragment comprises the carboxy-terminal portions (i.e., the CH2 and CH3 domains) of both H chains held together by disulfides. The effector functions of antibodies are determined by sequences in the Fc region. The Fc domain is the portion of the antibody recognized by cell receptors, such as the FcR, and to which the complement-activating protein, C1q, binds.

“Fv” is the minimum antibody fragment that contains a complete antigen-recognition and antigen-binding site. This fragment consists of a dimer of one heavy- and one light-chain variable region domain in tight, non-covalent association. From the folding of these two domains emanate six hypervariable loops (three loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

“Single-chain Fv” also abbreviated as “sFv” or “scFv” are antibody fragments that comprise the V_{H} and V_{L} antibody domains connected into a single polypeptide chain. Preferably, the sFv polypeptide further comprises a polypeptide linker between the V_{H} and V_{L} domains that enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994); Borrebaeck 1995, infra.

The term “diabodies” refers to small antibody fragments prepared by constructing sFv fragments (see preceding paragraph) with short linkers (about 5-10 residues) between the V_{H} and V_{L} domains such that inter-chain but not intra-chain pairing of the V domains is achieved, resulting in a bivalent fragment, i.e., fragment having two antigen-binding sites. Bispecific diabodies are heterodimers of two “crossover” sFv fragments in which the V_{H} and V_{L} domains of the two antibodies are present on different polypeptide chains. Diabodies are described more fully in, for example, EP 404,097; WO 93/11161; and Hollinger et al., Proc. Natl. Acad. Sci. USA, 90:6444-6448 (1993).

As used herein, the term “polyclonal antibody” refers to an antibody obtained from a population of antigen-specific antibodies that recognize more than one epitope of the specific antigen. “Antigen” or “immunogen” refers to a peptide, lipid, polysaccharide or polynucleotide which is recognized by the adaptive immune system of a host organism. Antigens may be self or non-self (i.e., foreign) molecules, where self antigens often include autoantigens that may be inappropriately recognized by the immune system as is characteristically seen in autoimmune disease. Examples of antigens include, but are not limited to, bacterial cell wall components, pollen, and Rh factor. The region of an antigen that is specifically recognized by a specific antibody is an “epitope” or “antigenic determinant”. A single antigen may have multiple epitopes.

Generation of polyclonal antibodies is well known in the art, as described, for example, in Weir, D. M., Handbook of Experimental Immunology, 1986, Blackwell Scientific, Boston, MA (USA). Briefly, an antigen against which specifically binding polyclonal antibodies are desired may be used to immunize a host animal, often in an immunization regimen comprising an initial primary immunization step which includes administering the antigen to the host, optionally along with an adjuvant (e.g., complete Freund's adjuvant), followed at various time intervals by one or more secondary booster immunization steps in which the antigen is again administered (optionally with an adjuvant, e.g., incomplete Freund's adjuvant). Serum samples collected from the host before, during, and after the immunization regimen may be tested in vitro for the presence of antibodies that specifically bind to the immunizing antigen. Structural (e.g., identification of isotype, molecular weight, and/or isoelectric focusing point diversity) and functional (e.g., identification of epitopes recognized) characterization of the antibodies present in the antisera so obtained may be conducted to confirm polyclonality of the antibodies present in the immunoglobulin fraction, according to conventional methodologies (e.g., Current Protocols in Immunology, 2005, John Wiley & Sons, NY; Weir, D. M., Handbook of Experimental Immunology, 1986, Blackwell Scientific, Boston). The polyclonal antibody-containing fraction may be enriched from an antiserum, for example, by molecular sizing for immunoglobulins (e.g., gel filtration), by preparative isoelectric focusing or by affinity isolation on immobilized Fc receptors (e.g., staphylococcal protein A or protein G, or synthetic Fc receptors), by affinity binding to immobilized antigen followed by elution of the antibodies with gentle reversible disruption of antigen-antibody binding (e.g., by changing pH or ionic strength), or by other conventional methodologies for antibody enrichment.

The term “monoclonal antibody” (mAb) as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations that include different antibodies directed against different epitopes, each monoclonal antibody is directed against a single epitope of the antigen. In addition to their specificity, the monoclonal antibodies are advantageous in that they may be synthesized uncontaminated by other antibodies. The modifier “monoclonal” is not to be construed as requiring production of the antibody by any particular method. For example, the monoclonal antibodies useful in the present disclosure may be prepared by the hybridoma methodology first described by Kohler et al., Nature, 256:495 (1975), or may be made using recombinant DNA methods in bacterial, eukaryotic animal or plant cells (see, e.g., U.S. Pat. No. 4,816,567). The “monoclonal antibodies” may also be isolated from phage antibody libraries using the techniques described in Clackson et al., Nature, 352:624-628 (1991) and Marks et al., J. Mol. Biol., 222:581-597 (1991), for example.

The monoclonal antibodies herein include “chimeric antibodies” in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see, U.S. Pat. Nos. 4,816,567; 5,530,101 and 7,498,415; and Morrison et al., Proc. Natl. Acad. Sci. USA, 81:6851-6855 (1984)). For example, chimeric antibodies may comprise human and non-human residues. Furthermore, chimeric antibodies may comprise residues that are not found in the recipient antibody or in the donor antibody. These modifications are made to further refine antibody performance. For further details, see Jones et al., Nature 321:522-525 (1986); Riechmann et al., Nature 332:323-329 (1988); and Presta, Curr. Op. Struct. Biol. 2:593-596 (1992). Chimeric antibodies also include primatized and humanized antibodies.

A “humanized antibody” is generally considered to be a human antibody that has one or more amino acid residues introduced into it from a source that is non-human. These non-human amino acid residues are typically taken from a variable domain. Humanization is traditionally performed following the method of Winter and co-workers (Jones et al., Nature, 321:522-525 (1986); Reichmann et al., Nature, 332:323-327 (1988); Verhoeyen et al., Science, 239:1534-1536 (1988)), by substituting non-human variable sequences for the corresponding sequences of a human antibody. Accordingly, such “humanized” antibodies are chimeric antibodies (U.S. Pat. Nos. 4,816,567; 5,530,101 and 7,498,415) wherein substantially less than an intact human variable domain has been substituted by the corresponding sequence from a non-human species. In some instances, a “humanized” antibody is one which is produced by a non-human cell or animal and comprises human sequences, e.g., H_{C} domains.

A “human antibody” is an antibody containing only sequences present in an antibody naturally produced by a human. However, as used herein, human antibodies may comprise residues or modifications not found in a naturally occurring human antibody, including those modifications and variant sequences described herein. These are typically made to further refine or enhance antibody performance. In some instances, human antibodies are produced by transgenic animals. For example, see U.S. Pat. Nos. 5,770,429; 6,596,541 and 7,049,426.

Antibody “effector functions” refer to those biological activities attributable to the Fc region (a native sequence Fc region or amino acid sequence variant Fc region) of an antibody, and vary with the antibody isotype. Examples of antibody effector functions include: C1q binding and complement dependent cytotoxicity; Fc receptor binding; antibody-dependent cell-mediated cytotoxicity (ADCC); phagocytosis; down regulation of cell surface receptors (e.g., B cell receptor); and B cell activation.

The phrase “functional fragment or analog” of an antibody is a compound having qualitative biological activity in common with a full-length antibody. For example, a functional fragment or analog of an anti-PeptideX2-13 antibody is one that can bind specifically to PeptideX2-13.

An antibody having a “biological characteristic” of a designated antibody is one that possesses one or more of the biological characteristics of that antibody which distinguish it from other antibodies. For example, in certain embodiments, an antibody with a biological characteristic of a designated antibody will bind the same epitope as that bound by the designated antibody and/or have a common effector function as the designated antibody.

As used herein, an antibody is said to be “immunospecific,” “specific for” or to “specifically bind” an antigen if it reacts at a detectable level with the antigen, preferably with an affinity constant, Kₐ of greater than or equal to about 10⁴ M⁻¹, or greater than or equal to about 10⁵ M⁻¹, greater than or equal to about 10⁶ M⁻¹, greater than or equal to about 10⁷ M¹, or greater than or equal to 10⁸ M⁻¹. Affinity of an antibody for its cognate antigen is also commonly expressed as a dissociation constant K_{D}, and in certain embodiments, a PeptideX2-specific antibody specifically binds to PeptideX2 if it binds with a K_{D} of less than or equal to 10⁴ M, less than or equal to about 10⁵ M, less than or equal to about 10⁶ M, less than or equal to 10⁷ M, or less than or equal to 10⁸ M. Affinities of antibodies can be readily determined using conventional techniques, for example, those described by Scatchard et al. (Ann. N.Y. Acad. Sci. USA 51:660 (1949)).

Binding properties of an antibody to antigens, cells or tissues thereof may generally be determined and assessed using immunodetection methods including, for example, immunofluorescence-based assays, such as immuno-histochemistry (IHC) and/or fluorescence-activated cell sorting (FACS).

“Carriers” as used herein include pharmaceutically acceptable carriers, excipients, or stabilizers that are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as polysorbate 20 (TWEEN™) polyethylene glycol (PEG), and poloxamers (PLURONICS™), and the like.

### Nucleic Acids and Polynucleotides

Immunomodulatory polypeptides as provided herein, and encoding nucleic acid molecules and vectors, may be isolated and/or purified, e.g., from their natural environment, in substantially pure or homogeneous form, or, in the case of nucleic acid, free or substantially free of nucleic acid or genes of origin other than the sequence encoding a polypeptide with the desired function. Nucleic acid may comprise DNA or RNA and may be wholly or partially synthetic. Reference to a nucleotide sequence as set out herein encompasses a DNA molecule with the specified sequence, and encompasses a RNA molecule with the specified sequence in which U is substituted for T, unless context requires otherwise.

The present disclosure thus further provides in certain embodiments an isolated nucleic acid encoding PeptideX2-13 (comprising the amino acid sequence set forth in SEQ ID NO: 3) or a polypeptide of 14, 15, 16, 17, 18, 19, 20, 21, 22, 23, 24, 25, 26, 27, 28, 29, 30 or 31 amino acids that includes the tridecameric sequence of PeptideX2-13 (SEQ ID NO: 3). In certain other embodiments, the present disclosure provides an isolated nucleic acid encoding a PeptideX2-13 variant (e.g., a variant as set forth in SEQ ID NO: 4, 5, 10, 11, 12, 13, 14, 15, 16, 17 or 22), or a polypeptide of 31, 30, 29, 28, 27, 26, 25, 24, 23, 22, 21, 20, 19, 18, 17, 16, 15, 14, or 13, optionally 12, 11, 10, 9, 8, 7, 6, or 5 amino acids in length and comprising the sequence of the variant.

Certain other embodiments additionally contemplate an antibody or antigen-binding fragment thereof that specifically binds to PeptideX2-13 or variants thereof as described herein, for instance, an antibody that may itself be an immunomodulatory polypeptide that competes with PeptideX2-13 or variants thereof for specific binding to a human neutrophil. Certain related embodiments may therefore contemplate a nucleic acid which codes for an anti-PeptideX2-13 immunoglobulin complementarity determining region (CDR) or heavy-chain variable (V_{H}) or light-chain variable (V_{L}) domain as described herein.

Nucleic acids include DNA and RNA. These and related embodiments may include polynucleotides encoding immunomodulatory polypeptides as described herein. The term “isolated polynucleotide” as used herein shall mean a polynucleotide of genomic, cDNA, or synthetic origin or some combination thereof, wherein by virtue of its origin the isolated polynucleotide (1) is not associated with all or a portion of a polynucleotide in which the isolated polynucleotide is found in nature, (2) is linked to a polynucleotide to which it is not linked in nature, or (3) does not occur in nature as part of a larger sequence.

Immunomodulatory polypeptides as provided herein, and encoding nucleic acid molecules and vectors, may be isolated and/or purified, e.g., from their natural environment, in substantially pure or homogeneous form, or, in the case of nucleic acid, free or substantially free of nucleic acid or genes of origin other than the sequence encoding a polypeptide with the desired function. Nucleic acid may comprise DNA or RNA and may be wholly or partially synthetic. Reference to a nucleotide sequence as set out herein encompasses a DNA molecule with the specified sequence, and encompasses a RNA molecule with the specified sequence in which U is substituted for T, unless context requires otherwise.

The term “operably linked” means that the components to which the term is applied are in a relationship that allows them to carry out their inherent functions under suitable conditions. For example, a transcription control sequence “operably linked” to a protein coding sequence is ligated thereto so that expression of the protein coding sequence is achieved under conditions compatible with the transcriptional activity of the control sequences.

The term “control sequence” as used herein refers to polynucleotide sequences that can affect expression, processing or intracellular localization of coding sequences to which they are ligated or operably linked. The nature of such control sequences may depend upon the host organism. In particular embodiments, transcription control sequences for prokaryotes may include a promoter, ribosomal binding site, and transcription termination sequence. In other particular embodiments, transcription control sequences for eukaryotes may include promoters comprising one or a plurality of recognition sites for transcription factors, transcription enhancer sequences, transcription termination sequences and polyadenylation sequences. In certain embodiments, “control sequences” can include leader sequences and/or fusion partner sequences.

The term “polynucleotide” as referred to herein means single-stranded or double-stranded nucleic acid polymers. In certain embodiments, the nucleotides comprising the polynucleotide can be ribonucleotides or deoxyribonucleotides or a modified form of either type of nucleotide. Such modifications may include base modifications such as bromouridine, ribose modifications such as arabinoside and 2′,3′-dideoxyribose and internucleotide linkage modifications such as phosphorothioate, phosphorodithioate, phosphoroselenoate, phosphorodiselenoate, phosphoroanilothioate, phoshoraniladate and phosphoroamidate. The term “polynucleotide” specifically includes single and double stranded forms of DNA.

The term “naturally occurring nucleotides” includes deoxyribonucleotides and ribonucleotides. The term “modified nucleotides” includes nucleotides with modified or substituted sugar groups and the like. The term “oligonucleotide linkages” includes oligonucleotide linkages such as phosphorothioate, phosphorodithioate, phosphoroselenoate, phosphorodiselenoate, phosphoroanilothioate, phoshoraniladate, phosphoroamidate, and the like. See, e.g., LaPlanche et al., 1986, Nucl. Acids Res., 14:9081; Stec et al., 1984, J. Am. Chem. Soc., 106:6077; Stein et al., 1988, Nucl. Acids Res., 16:3209; Zon et al., 1991, Anti-Cancer Drug Design, 6:539; Zon et al., 1991, OLIGONUCLEOTIDES AND ANALOGUES: A PRACTICAL APPROACH, pp. 87-10⁸ (F. Eckstein, Ed.), Oxford University Press, Oxford England; Stec et al., U.S. Pat. No. 5,151,510; Uhlmann and Peyman, 1990, Chemical Reviews, 90:543, the disclosures of which are hereby incorporated by reference for any purpose. An oligonucleotide can include a detectable label to enable detection of the oligonucleotide or hybridization thereof.

The term “vector” is used to refer to any molecule (e.g., nucleic acid, plasmid, or virus) used to transfer coding information to a host cell. The term “expression vector” refers to a vector that is suitable for transformation of a host cell and contains nucleic acid sequences that direct and/or control expression of inserted heterologous nucleic acid sequences. Expression includes, but is not limited to, processes such as transcription, translation, and RNA splicing, if introns are present.

As will be understood by those skilled in the art, polynucleotides may include genomic sequences, extra-genomic and plasmid-encoded sequences and smaller engineered gene segments that express, or may be adapted to express, proteins, polypeptides, peptides and the like. Such segments may be naturally isolated or modified synthetically by the skilled person.

As will be also recognized by the skilled artisan, polynucleotides may be single-stranded (coding or antisense) or double-stranded, and may be DNA (genomic, cDNA or synthetic) or RNA molecules. RNA molecules may include HnRNA molecules, which contain introns and correspond to a DNA molecule in a one-to-one manner, and mRNA molecules, which do not contain introns. Additional coding or non-coding sequences may, but need not, be present within a polynucleotide according to the present disclosure, and a polynucleotide may, but need not, be linked to other molecules and/or support materials. Polynucleotides may comprise a native sequence or may comprise a sequence that encodes a variant or derivative of such a sequence.

Therefore, according to these and related embodiments, the present disclosure also provides polynucleotides encoding the immunomodulatory polypeptides (e.g., PeptideX2-13 (SEQ ID NO: 3) or a variant thereof (e.g., SEQ ID NO: 4, 5, 10, 11, 12, 13, 14, 15, 16, 17, or 22)], which as provided herein may comprise 31, 30, 29, 28, 27, 26, 25, 24, 23, 22, 21, 20, 19, 18, 17, 16, 15, 14, or 13, optionally 12, 11, 10, 9, 8, 7, 6, or 5 amino acids. In certain embodiments, polynucleotides are provided that comprise some or all of a polynucleotide sequence encoding a polypeptide or peptide as described herein and complements of such polynucleotides.

In other related embodiments, polynucleotide variants may have substantial identity to a polynucleotide sequence encoding an immunomodulatory polypeptide described herein. For example, a polynucleotide may be a polynucleotide comprising at least 70% sequence identity, preferably at least 75%, 80%, 85%, 90%, 95%, 96%, 97%, 98%, or 99% or higher, sequence identity compared to a reference polynucleotide sequence such as a sequence encoding an antibody described herein, using the methods described herein, (e.g., BLAST analysis using standard parameters, as described below). One skilled in this art will recognize that these values can be appropriately adjusted to determine corresponding identity of proteins encoded by two nucleotide sequences by considering codon degeneracy, amino acid similarity, reading frame positioning and the like.

Typically, polynucleotide variants will contain one or more substitutions, additions, deletions and/or insertions, preferably such that the binding affinity of the antibody encoded by the variant polynucleotide is not substantially diminished relative to an antibody encoded by a polynucleotide sequence specifically set forth herein.

In certain other related embodiments, polynucleotide fragments may comprise or consist essentially of various lengths of contiguous stretches of sequence identical to or complementary to a sequence encoding an immunomodulatory polypeptide as described herein. For example, polynucleotides are provided that comprise or consist essentially of at least about 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100, 110, 120, 130, 140, 150, 200, 300, 400, 500 or 1000 or more contiguous nucleotides of a sequences the encodes an immunomodulatory polypeptide, or variant thereof, disclosed herein as well as all intermediate lengths there between. It will be readily understood that “intermediate lengths”, in this context, means any length between the quoted values, such as 50, 51, 52, 53, etc.; 100, 101, 102, 103, etc.; 150, 151, 152, 153, etc.; including all integers through 200-500; 500-1,000, and the like. A polynucleotide sequence as described here may be extended at one or both ends by additional nucleotides not found in the native sequence. This additional sequence may consist of 1, 2, 3, 4, 5, 6, 7, 8, 9, 10, 11, 12, 13, 14, 15, 16, 17, 18, 19, or 20 nucleotides at either end of a polynucleotide encoding an immunomodulatory polypeptide described herein or at both ends of a polynucleotide encoding an immunomodulatory polypeptide described herein.

In another embodiment, polynucleotides are provided that are capable of hybridizing under moderate to high stringency conditions to a polynucleotide sequence encoding an immunomodulatory polypeptide, or variant thereof, provided herein, or a fragment thereof, or a complementary sequence thereof. Hybridization techniques are well known in the art of molecular biology. For purposes of illustration, suitable moderately stringent conditions for testing the hybridization of a polynucleotide as provided herein with other polynucleotides include prewashing in a solution of 5×SSC, 0.5% SDS, 1.0 mM EDTA (pH 8.0); hybridizing at 50° C.-60° C., 5×SSC, overnight; followed by washing twice at 65° C. for 20 minutes with each of 2X, 0.5X and 0.2×SSC containing 0.1% SDS. One skilled in the art will understand that the stringency of hybridization can be readily manipulated, such as by altering the salt content of the hybridization solution and/or the temperature at which the hybridization is performed. For example, in another embodiment, suitable highly stringent hybridization conditions include those described above, with the exception that the temperature of hybridization is increased, e.g., to 60° C.-65° C. or 65° C.70° C.

In certain embodiments, the polynucleotides described above, e.g., polynucleotide variants, fragments and hybridizing sequences, encode immunomodulatory polypeptides that bind neutrophils. In other embodiments, such polynucleotides encode immunomodulatory polypeptides, or variants thereof, that bind to neutrophils at least about 50%, at least about 70%, and in certain embodiments, at least about 90% as well as an immunomodulatory polypeptide sequence specifically set forth herein (e.g., PeptideX2-13). In further embodiments, such polynucleotides encode immunomodulatory polypeptides, or variants thereof, that bind to neutrophils with greater affinity than the immunomodulatory polypeptides set forth herein, for example, that bind quantitatively at least about 10⁵%, 106%, 107%, 10⁸%, 109%, or 110% as well as an immunomodulatory peptide sequence specifically set forth herein.

As described elsewhere herein, determination of the three-dimensional structures of representative polypeptides (e.g., PeptideX2-13 (SEQ ID NO: 3) or variants thereof) may be made through routine methodologies such that substitution, addition, deletion or insertion of one or more amino acids with selected natural or non-natural amino acids can be virtually modeled for purposes of determining whether a so derived structural variant retains the space-filling properties of presently disclosed species. A variety of computer programs are known to the skilled artisan for determining appropriate amino acid substitutions (or appropriate polynucleotides encoding the amino acid sequence) within an antibody such that, for example, affinity is maintained or better affinity is achieved.

The polynucleotides described herein, or fragments thereof, regardless of the length of the coding sequence itself, may be combined with other DNA sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant

DNA protocol. For example, illustrative polynucleotide segments with total lengths of about 10,000, about 5000, about 3000, about 2,000, about 1,000, about 500, about 200, about 100, about 50 base pairs in length, and the like, (including all intermediate lengths) are contemplated to be useful.

When comparing polynucleotide sequences, two sequences are said to be “identical” if the sequence of nucleotides in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, WI), using default parameters. This program embodies several alignment schemes described in the following references: Dayhoff, M. O. (1978) A model of evolutionary change in proteins-Matrices for detecting distant relationships. In Dayhoff, M. O. (ed.) Atlas of Protein Sequence and Structure, National Biomedical Research Foundation, Washington DC Vol. 5, Suppl. 3, pp. 345-358; Hein J., Unified Approach to Alignment and Phylogenes, pp. 626-645 (1990); Methods in Enzymology vol. 183, Academic Press, Inc., San Diego, CA; Higgins, D. G. and Sharp, P. M., CABIOS 5:151-153 (1989); Myers, E. W. and Muller W., CABIOS 4:11-17 (1988); Robinson, E. D., Comb. Theor 11:10⁵ (1971); Santou, N. Nes, M., Mol. Biol. Evol. 4:406-425 (1987); Sneath, P. H. A. and Sokal, R. R., Numerical Taxonomy—the Principles and Practice of Numerical Taxonomy, Freeman Press, San Francisco, CA (1973); Wilbur, W. J. and Lipman, D. J., Proc. Natl. Acad., Sci. USA 80:726-730 (1983).

Alternatively, optimal alignment of sequences for comparison may be conducted by the local identity algorithm of Smith and Waterman, Add. APL. Math 2:482 (1981), by the identity alignment algorithm of Needleman and Wunsch, J. Mol. Biol. 48:443 (1970), by the search for similarity methods of Pearson and Lipman, Proc. Natl. Acad. Sci. USA 85:2444 (1988), by computerized implementations of these algorithms (GAP, BESTFIT, BLAST, FASTA, and TFASTA in the Wisconsin Genetics Software Package, Genetics Computer Group (GCG), 575 Science Dr., Madison, WI), or by inspection.

One preferred example of algorithms that are suitable for determining percent sequence identity and sequence similarity are the BLAST and BLAST 2.0 algorithms, which are described in Altschul et al., Nucl. Acids Res. 25:3389-3402 (1977), and Altschul et al., J. Mol. Biol. 215:403-410 (1990), respectively. BLAST and BLAST 2.0 can be used, for example with the parameters described herein, to determine percent sequence identity among two or more the polynucleotides. Software for performing BLAST analyses is publicly available through the National Center for Biotechnology Information. In one illustrative example, cumulative scores can be calculated using, for nucleotide sequences, the parameters M (reward score for a pair of matching residues; always >0) and N (penalty score for mismatching residues; always <0). Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLASTN program (for nucleotide sequences) uses as defaults a wordlength (W) of 11, and expectation (E) of 10, and the BLOSUM62 scoring matrix (see Henikoff and Henikoff, Proc. Natl. Acad. Sci. USA 89:10915 (1989)) alignments, (B) of 50, expectation (E) of 10, M=5, N=−4 and a comparison of both strands.

In certain embodiments, the “percentage of sequence identity” is determined by comparing two optimally aligned sequences over a window of comparison of at least 20 positions, wherein the portion of the polynucleotide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases occurs in both sequences to yield the number of matched positions, dividing the number of matched positions by the total number of positions in the reference sequence (i.e., the window size) and multiplying the results by 100 to yield the percentage of sequence identity.

It will be appreciated by those of ordinary skill in the art that, as a result of the degeneracy of the genetic code, there are many nucleotide sequences that encode an immunomodulatory peptide as described herein, or an antibody that specifically binds to such a peptide, as described herein. Some of these polynucleotides bear minimal sequence identity to the nucleotide sequence of the native or original polynucleotide sequence that encode immunomodulatory polypeptides described herein. Nonetheless, polynucleotides that vary due to differences in codon usage are expressly contemplated by the present disclosure. In certain embodiments, sequences that have been codon-optimized for mammalian expression are specifically contemplated.

Therefore, in another embodiment of the present disclosure, a mutagenesis approach, such as site-specific mutagenesis, may be employed for the preparation of variants and/or derivatives of the immunomodulatory polypeptides described herein. By this approach, specific modifications in a polypeptide sequence can be made through mutagenesis of the underlying polynucleotides that encode them. These techniques provide a straightforward approach to prepare and test sequence variants, for example, incorporating one or more of the foregoing considerations, by introducing one or more nucleotide sequence changes into the polynucleotide.

Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Mutations may be employed in a selected polynucleotide sequence to improve, alter, decrease, modify, or otherwise change the properties of the polynucleotide itself, and/or alter the properties, activity, composition, stability, or primary sequence of the encoded polypeptide.

In certain embodiments, the mutagenesis of the polynucleotide sequences that encode an immunomodulatory polypeptide disclosed herein, or a variant thereof, to alter one or more properties of the encoded polypeptide, such as the binding affinity of the peptide or the variant thereof, or the immunosuppressive or immunostimulatory effects, is contemplated. The techniques of site-specific mutagenesis are well-known in the art and are widely used to create variants of both polypeptides and polynucleotides. For example, site-specific mutagenesis is often used to alter a specific portion of a DNA molecule. In such embodiments, a primer comprising typically about 14 to about 25 nucleotides or so in length is employed, with about 5 to about 10 residues on both sides of the junction of the sequence being altered.

As will be appreciated by those of skill in the art, site-specific mutagenesis techniques have often employed a phage vector that exists in both a single stranded and double stranded form. Typical vectors useful in site-directed mutagenesis include vectors such as the M13 phage. These phages are commercially available, and their use is generally well-known to those skilled in the art. Double-stranded plasmids are also routinely employed in site directed mutagenesis that eliminates the step of transferring the gene of interest from a plasmid to a phage.

In general, site-directed mutagenesis in accordance herewith is performed by first obtaining a single-stranded vector or melting apart of two strands of a double-stranded vector that includes within its sequence a DNA sequence that encodes the desired peptide. An oligonucleotide primer bearing the desired mutated sequence is prepared, generally synthetically. This primer is then annealed with the single-stranded vector and subjected to DNA polymerizing enzymes such as E. coli polymerase I Klenow fragment to complete the synthesis of the mutation-bearing strand. Thus, a heteroduplex is formed wherein one strand encodes the original non-mutated sequence and the second strand bears the desired mutation. This heteroduplex vector is then used to transform appropriate cells, such as E. coli cells, and clones are selected which include recombinant vectors bearing the mutated sequence arrangement.

The preparation of sequence variants of the selected peptide-encoding DNA segments using site-directed mutagenesis provides a means of producing potentially useful species and is not meant to be limiting as there are other ways in which sequence variants of peptides and the DNA sequences encoding them may be obtained. For example, recombinant vectors encoding the desired peptide sequence may be treated with mutagenic agents, such as hydroxylamine, to obtain sequence variants. Specific details regarding these methods and protocols are found in the teachings of Maloy et al., 1994; Segal, 1976; Prokop and Bajpai, 1991; Kuby, 1994; and Maniatis et al., 1982, each incorporated herein by reference, for that purpose.

As used herein, the term “oligonucleotide directed mutagenesis procedure” refers to template-dependent processes and vector-mediated propagation which result in an increase in the concentration of a specific nucleic acid molecule relative to its initial concentration, or in an increase in the concentration of a detectable signal, such as amplification. As used herein, the term “oligonucleotide directed mutagenesis procedure” is intended to refer to a process that involves the template-dependent extension of a primer molecule. The term template dependent process refers to nucleic acid synthesis of an RNA or a DNA molecule wherein the sequence of the newly synthesized strand of nucleic acid is dictated by the well-known rules of complementary base pairing (see, for example, Watson, 1987). Typically, vector mediated methodologies involve the introduction of the nucleic acid fragment into a DNA or RNA vector, the clonal amplification of the vector, and the recovery of the amplified nucleic acid fragment. Examples of such methodologies are provided by U.S. Pat. No. 4,237,224, specifically incorporated herein by reference in its entirety.

In another approach to produce polypeptide variants, recursive sequence recombination, as described in U.S. Pat. No. 5,837,458, may be employed. In this approach, iterative cycles of recombination and screening or selection are performed to “evolve” individual polynucleotide variants having, for example, increased binding affinity. Certain embodiments also provide constructs in the form of plasmids, vectors, transcription or expression cassettes which comprise at least one polynucleotide as described herein.

According to certain related embodiments there is provided a recombinant host cell which comprises one or more constructs as described herein; a nucleic acid encoding immunomodulatory polypeptide or variant thereof; and a method of producing of the encoded product, which method comprises expression from encoding nucleic acid therefor. Expression may conveniently be achieved by culturing under appropriate conditions recombinant host cells containing the nucleic acid. Following production by expression, an immunomodulatory polypeptide may be isolated and/or purified using any suitable technique, and then used as desired.

Systems for cloning and expression of a polypeptide in a variety of different host cells are well known. Suitable host cells include bacteria, mammalian cells, yeast and baculovirus systems. Mammalian cell lines available in the art for expression of a heterologous polypeptide include Chinese hamster ovary cells, HeLa cells, baby hamster kidney cells, NSO mouse melanoma cells and many others. A common, preferred bacterial host is E. coli.

The expression of peptides in prokaryotic cells such as E. coli is well established in the art. For a review, see for example Pluckthun, A. Bio/Technology 9:545-551 (1991). Expression in eukaryotic cells in culture is also available to those skilled in the art as an option for production of immunomodulatory polypeptides, see recent reviews, for example Ref, M. E. (1993) Curr. Opinion Biotech. 4:573-576; Trill J. J. et al. (1995) Curr. Opinion Biotech 6:553-560.

Suitable vectors can be chosen or constructed, containing appropriate regulatory sequences, including promoter sequences, terminator sequences, polyadenylation sequences, enhancer sequences, marker genes and other sequences as appropriate. Vectors may be plasmids, viral e.g. phage, or phagemid, as appropriate. For further details see, for example, Molecular Cloning: a Laboratory Manual: 2nd edition, Sambrook et al., 1989, Cold Spring Harbor Laboratory Press. Many known techniques and protocols for manipulation of nucleic acid, for example in preparation of nucleic acid constructs, mutagenesis, sequencing, introduction of DNA into cells and gene expression, and analysis of proteins, are described in detail in Current Protocols in Molecular Biology, Second Edition, Ausubel et al. eds., John Wiley & Sons, 1992, or subsequent updates thereto.

The term “host cell” is used to refer to a cell into which has been introduced, or which is capable of having introduced into it, a nucleic acid sequence encoding one or more of the herein described immunomodulatory polypeptides, and which further expresses or is capable of expressing a selected gene of interest, such as a gene encoding any herein described immunomodulatory polypeptide. The term includes the progeny of the parent cell, whether or not the progeny is identical in morphology or in genetic make-up to the original parent, so long as the selected gene is present. Accordingly, there is also contemplated a method comprising introducing such nucleic acid into a host cell. The introduction may employ any available technique. For eukaryotic cells, suitable techniques may include calcium phosphate transfection, DEAE-Dextran, electroporation, liposome-mediated transfection and transduction using retrovirus or other virus, e.g., vaccinia or, for insect cells, baculovirus. For bacterial cells, suitable techniques may include calcium chloride transformation, electroporation and transfection using bacteriophage. The introduction may be followed by causing or allowing expression from the nucleic acid, e.g., by culturing host cells under conditions for expression of the gene. In one embodiment, the nucleic acid is integrated into the genome (e.g., chromosome) of the host cell. Integration may be promoted by inclusion of sequences which promote recombination with the genome, in accordance—with standard techniques.

The present disclosure also provides, in certain embodiments, a method which comprises using a construct as stated above in an expression system to express a particular polypeptide such as an immunomodulatory polypeptide as described herein. The term “transduction” is used to refer to the transfer of genes from one bacterium to another, usually by a phage. “Transduction” also refers to the acquisition and transfer of eukaryotic cellular sequences by retroviruses. The term “transfection” is used to refer to the uptake of foreign or exogenous DNA by a cell, and a cell has been “transfected” when the exogenous DNA has been introduced inside the cell membrane. A number of transfection techniques are well known in the art and are disclosed herein. See, e.g., Graham et al., 1973, Virology 52:456; Sambrook et al., 2001, Molecular Cloning: A Laboratory Manual, Cold Spring Harbor Laboratories; Davis et al., 1986, Basic Methods in Molecular Biology, Elsevier; and Chu et al., 1981, Gene 13:197. Such techniques can be used to introduce one or more exogenous DNA moieties into suitable host cells.

The term “transformation” as used herein refers to a change in a cell's genetic characteristics, and a cell has been transformed when it has been modified to contain a new DNA. For example, a cell is transformed where it is genetically modified from its native state. Following transfection or transduction, the transforming DNA may recombine with that of the cell by physically integrating into a chromosome of the cell or may be maintained transiently as an episomal element without being replicated or may replicate independently as a plasmid. A cell is considered to have been stably transformed when the DNA is replicated with the division of the cell.

The term “naturally occurring” or “native” when used in connection with biological materials such as nucleic acid molecules, polypeptides, host cells, and the like, refers to materials which are found in nature and are not manipulated by a human. Similarly, “non-naturally occurring” or “non-native” as used herein refers to a material that is not found in nature or that has been structurally modified or synthesized by a human.

Certain embodiments contemplated herein include antisense-based nucleic acid technologies that may be implemented in a manner that specifically alters (e.g., increases or decreases in a statistically significant manner) expression of a PeptideX2-13-encoding polynucleotide, or of a polynucleotide that encodes a variant of PeptideX2-13 as provided herein. Such antisense-based technologies include RNA interference, ribozymes and antisense nucleic acids.

RNA interference (RNAi) is a polynucleotide sequence-specific, post-transcriptional gene silencing mechanism effected by double-stranded RNA that results in degradation of a specific messenger RNA (mRNA), thereby reducing the expression of a desired target polypeptide encoded by the mRNA (see, e.g., WO 99/32619; WO 01/75164; U.S. Pat. No. 6,506,559; Fire et al., Nature 391:806-11 (1998); Sharp, Genes Dev. 13:139-41 (1999); Elbashir et al. Nature 411:494-98 (2001); Harborth et al., J. Cell Sci. 114:4557-65 (2001)). RNAi is mediated by double-stranded polynucleotides as also described herein below, for example, double-stranded RNA (dsRNA), having sequences that correspond to exonic sequences encoding portions of the polypeptides for which expression is compromised. RNAi reportedly is not effected by double-stranded RNA polynucleotides that share sequence identity with intronic or promoter sequences (Elbashir et al., 2001). RNAi pathways have been best characterized in Drosophila and Caenorhabditis elegans, but “small interfering RNA” (siRNA) polynucleotides that interfere with expression of specific polypeptides in higher eukaryotes such as mammals (including humans) have also been described (e.g., Tuschl, 2001 Chembiochem. 2:239-245; Sharp, 2001 Genes Dev. 15:485; Bernstein et al., 2001 RNA 7:1509; Zamore, 2002 Science 296:1265; Plasterk, 2002 Science 296:1263; Zamore 2001 Nat. Struct. Biol. 8:746; Matzke et al., 2001 Science 293:1080; Scadden et al., 2001 EMBO Rep. 2:1107) and subsequently elaborated upon. According to a current non-limiting model, the RNAi pathway is initiated by ATP-dependent, processive cleavage of long dsRNA into double-stranded fragments of about 18-27 (e.g., 19, 20, 21, 22, 23, 24, 25, 26, etc.) nucleotide base pairs in length, called small interfering RNAs (siRNAs) (see review by Hutvagner et al., Curr. Opin. Gen. Dev. 12:225-32 (2002); Elbashir et al., 2001; Nykänen et al., Cell 107:309-21 (2001); Zamore et al., Cell 101:25-33 (2000); Bass, Cell 101:235-38 (2000)). In Drosophila, an enzyme known as “Dicer” cleaves the longer double-stranded RNA into siRNAs; Dicer belongs to the RNase III family of dsRNA-specific endonucleases (WO 01/68836; Bernstein et al., Nature 409:363-66 (2001)). Further according to this non-limiting model, the siRNA duplexes are incorporated into a protein complex, followed by ATP-dependent unwinding of the siRNA, which then generates an active RNA-induced silencing complex (RISC) (WO 01/68836). The complex recognizes and cleaves a target RNA that is complementary to the guide strand of the siRNA, thus interfering with expression of a specific protein (Hutvagner et al., supra).

In C. elegans and Drosophila, RNAi may be mediated by long double-stranded RNA polynucleotides (WO 99/32619; WO 01/75164; Fire et al., 1998; Clemens et al., Proc. Natl. Acad. Sci. USA 97:6499-6503 (2000); Kisielow et al., Biochem. J. 363:1-5 (2002); see also WO 01/92513 (RNAi-mediated silencing in yeast)). In mammalian cells, however, transfection with long dsRNA polynucleotides (i.e., greater than 30 base pairs) leads to activation of a non-specific sequence response that globally blocks the initiation of protein synthesis and causes mRNA degradation (Bass, Nature 411:428-29 (2001)). Transfection of human and other mammalian cells with double-stranded RNAs of about 18-27 nucleotide base pairs in length interferes in a sequence-specific manner with expression of particular polypeptides encoded by messenger RNAs (mRNA) containing corresponding nucleotide sequences (WO 01/75164; Elbashir et al., 2001; Elbashir et al., Genes Dev. 15:188-200 (2001)); Harborth et al., J. Cell Sci. 114:4557-65 (2001); Carthew et al., Curr. Opin. Cell Biol. 13:244-48 (2001); Mailand et al., Nature Cell Biol. Advance Online Publication (Mar. 18, 2002); Mailand et al. 2002 Nature Cell Biol. 4:317). siRNA polynucleotides may offer certain advantages over other polynucleotides known to the art for use in sequence-specific alteration or modulation of gene expression to yield altered levels of an encoded polypeptide product. These advantages include lower effective siRNA polynucleotide concentrations, enhanced siRNA polynucleotide stability, and shorter siRNA polynucleotide oligonucleotide lengths relative to such other polynucleotides (e.g., antisense, ribozyme or triplex polynucleotides).

By way of a brief background, “antisense” polynucleotides bind in a sequence-specific manner to target nucleic acids, such as mRNA or DNA, to prevent transcription of DNA or translation of the mRNA (see, e.g., U.S. Pat. Nos. 5,168,053; 5,190,931; U.S.

U.S. Pat. Nos. 5,135,917; 5,087,617; see also, e.g., Clusel et al., 1993 Nucl. Acids Res. 21:3405-11, describing “dumbbell” antisense oligonucleotides). “Ribozyme” polynucleotides can be targeted to any RNA transcript and are capable of catalytically cleaving such transcripts, thus impairing translation of mRNA (see, e.g., U.S. Pat. Nos. 5,272,262; 5,144,019; and 5,168,053, 5,180,818, 5,116,742 and 5,093,246; U.S. 2002/193579). “Triplex” DNA molecules refers to single DNA strands that bind duplex DNA to form a colinear triplex molecule, thereby preventing transcription (see, e.g., U.S. Pat. No. 5,176,996, describing methods for making synthetic oligonucleotides that bind to target sites on duplex DNA). Such triple-stranded structures are unstable and form only transiently under physiological conditions.

Because single-stranded polynucleotides do not readily diffuse into cells and are therefore susceptible to nuclease digestion, development of single-stranded DNA for antisense or triplex technologies often requires chemically modified nucleotides to improve stability and absorption by cells. siRNAs, by contrast, are readily taken up by intact cells, are effective at interfering with the expression of specific polypeptides at concentrations that are several orders of magnitude lower than those required for either antisense or ribozyme polynucleotides, and do not require the use of chemically modified nucleotides.

It will be appreciated that the practice of the several embodiments of the present disclosure will employ, unless indicated specifically to the contrary, conventional methods in virology, immunology, microbiology, molecular biology and recombinant DNA techniques that are within the skill of the art, and many of which are described below for the purpose of illustration. Such techniques are explained fully in the literature. See, e.g., Current Protocols in Molecular Biology and/or Current Protocols in Immunology, John Wiley & Sons, New York, NY (2009, 2015); Ausubel et al., Short Protocols in Molecular Biology, 3rd ed., Wiley & Sons, 1995; Sambrook and Russell, Molecular Cloning: A Laboratory Manual (3rd Edition, 2001); Maniatis et al. Molecular Cloning: A Laboratory Manual (1982); DNA Cloning: A Practical Approach, vol. I & II (D. Glover, ed.); Oligonucleotide Synthesis (N. Gait, ed., 1984); Nucleic Acid Hybridization (B. Hames & S. Higgins, eds., 1985); Transcription and Translation (B. Hames & S. Higgins, eds., 1984); Animal Cell Culture (R. Freshney, ed., 1986); Perbal, A Practical Guide to Molecular Cloning (1984) and other like references.

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. These and related techniques and procedures may be generally performed according to conventional methods well known in the art and as described in various general and more specific references that are cited and discussed throughout the present specification. Unless specific definitions are provided, the nomenclature utilized in connection with, and the laboratory procedures and techniques of, molecular biology, analytical chemistry, synthetic organic chemistry, and medicinal and pharmaceutical chemistry described herein are those well known and commonly used in the art. Standard techniques may be used for recombinant technology, molecular biological, microbiological, chemical syntheses, chemical analyses, pharmaceutical preparation, formulation, and delivery, and treatment of patients.

### Compositions and Methods of Use

The present disclosure provides compositions comprising an immunomodulatory polypeptide disclosed herein, an antibody as provided herein that specifically binds to such an immunomodulatory polypeptide, and administration of such compositions in a variety of therapeutic settings.

Certain presently disclosed embodiments contemplate exploitation of the herein described weak TLR2 agonist activity of the present immunomodulatory polypeptides comprising or consisting of SEQ ID NO: 3, 4, 5, 10, 11, 12, 13, 14, 15, 16, 17 or 22. For instance, such TLR2 and/or TLR4 agonist activity may be useful for treating sepsis by provoking a mild, as opposed to an excessive hyperinflammatory reaction that characterizes sepsis, without driving the immune system to the immunosuppressed state that otherwise often subsequently predominates later stages of sepsis. As also noted above, there is a known role of Toll like receptors (TLRs) in numerous inflammation-associated diseases, disorders, and conditions, including atherosclerosis and uveitis, including a number of autoimmune inflammatory diseases such as rheumatoid arthritis (e.g., Seibl et al 2003, Ospelt et al 2004, Ultaigh et al 2011, Lacerte et al 2016, McGarry et al 2017).

As shown in Table 1, TLR2 appears to be a key component of the inflammatory process in RA, Crohn's disease, atherosclerosis, and certain types of tissue injury- and therefore represents, according to non-limiting theory, a possible target for treatment with one or more of the herein disclosed immunomodulatory polypeptides, such as a polypeptide comprising PeptideX2-13 (SEQ ID NO:3) or one of its variants.

Historically, TLRs have been shown to be instrumental in the activation of the innate immune system and represent an important link between the innate and the acquired immune systems (Kawasaki et al 2014). TLRs represent pattern recognition receptors (PRRs) which identify pathogen associated molecular patterns (PAMPs) such as LPS (lipopolysaccharide found in gram negative bacteria), and lipotechoic acids (found in gram positive bacteria), and other microbial membrane components (Kawasaki et al 2014, Drexler et al 2006). Intracellular TLRs recognize nucleic acids derived from viruses and bacteria and thereby facilitate the immune response to viral infections and certain types of bacterial infections. Certain TLRs have been shown to be activated in autoimmune inflammatory diseases, probably because certain cellular products (such as heat shock proteins (HSP), and nuclear high mobility group box 1 (HMGB-1) proteins) are released during tissue damage and may be part of the feedback loop that perpetuates the inflammatory process in an autoimmune disease (Table 1) (Drexler et al 2006).

TABLE 1

| | TLRs | | |
|---|---|---|---|
| | associated with | Possible ligands | References |
| Disease | this disease | binding to the TLRs | (see “Additional References”, infra) |
| Rheumatoid | TLR2, TLR3, | HSP22, HMGB-1, | Radstake et al, Siebl et al, Iwahashi et al, |
| arthritis | TLR4, TLR9 | RNA, bacterial DNA | Roelofs et al, Andersson et al, Fiuza et al, |
| | | | Park et al, Park et al, van der Heijden et |
| | | | al, Schumacher et al, Gerard et al, |
| | | | Kempsell et al, Chen et al, Scherr et al, |
| | | | Scott et al, Yu et al, Taniguchi et al, |
| | | | Cromartie et al, Joosten et al, Pierer et al, |
| | | | Liu et al, Choe et al, Brentano et al, |
| | | | Roelofs et al, Kyburz et al, Wang et al, |
| | | | Kokkola et al, Kilding et al, Lamb et al, |
| | | | Sanchez et al, McCarty et al, Bieber et al, |
| | | | Biu-Bryan et al, Liu-Bryan et al, |
| | | | Martinon et al, Ting et al, Martinon et al, |
| | | | Martinon et al, Mariathasan et al |
| Crohn's | TLR2, TLR5, | Microbial antigens, | Hugot et al, Ogura et al, Inohara et al, |
| disease | TLR9 | bacterial DNA | Girardin et al, van Heel et al, Watanabe et |
| | | | al, Torok et al, Gewirtz et al |
| Atherosclerosis | TLR2, TLR4 | Fatty acids | Libby, Lee et al, Lee et al, Miller et al, |
| | | | Walton et al, Reddick et al, Michelsen et |
| | | | al, Wright et al, Bjorkbacka et al, Kirii et |
| | | | al, Elhage et al, Nishina et al, Kiechl et al, |
| | | | Ameziane et al, Netea et al |
| Tissue damage | TLR2, TLR4 | Hyaluronic acid, | Radoff et al, Jiang et al |
| | | microbial antigens | |

For example, in rheumatoid arthritis, once the inflammatory process results in tissue injury and joint damage, these and potentially other cellular products (known as DAMPs or danger associated molecular patterns) may activate the immune system via TLRs and thereby perpetuate the inflammatory process (Drexler et al 2006).

Reduced severity of streptococcal wall (SCW) induced arthritis has been demonstrated in TLR2 or MyD88 knockout mice, for example, indicating a role for TLR2 in this animal model of arthritis (Joosten et al 2003, Pierer et al 2004). Another demonstrated approach to blocking TLR2 activity employed a neutralizing antibody, which has been shown to prevent sepsis induced by TLR2 ligands (Meng et al).

Further evidence of a TLR2 role in RA may be found in the observation that TLR2 activation induced cellular migration and activation of inflammatory molecules found in RA (McGarry et al 2015). Increased TLR2 expression has also been reported in the synovium of RA patients (Siebl et al 2003) and may contribute to the mechanism of eventual joint damage in RA (Ospelt et al 2004, and Lacerte et al 2016). Blockade of TLR2 pathways has been shown to impair the inflammatory process in RA (McGarry et al 2017). In particular, TLR2 blockade with an anti-TLR2 antibody (OPN301) resulted in a decreased release of inflammatory cytokines from synovial tissue explants obtained from RA patients (Ultaigh et al 2011).

Accordingly and in view of the present disclosure, certain contemplated embodiments provide compositions and methods for treating TLR-mediated disorders such as sepsis, atherosclerosis, uveitis, autoimmune diseases including rheumatoid arthritis (RA), Crohn's disease, and other autoimmune diseases, graft rejection, GVHD, and also including TLR-mediated cancer, preeclampsia, HELLP, and other inflammation-associated conditions including certain types of tissue damage.

Administration of the immunomodulatory polypeptides, or antibodies specific therefor, described herein, in pure form or in an appropriate pharmaceutical composition, can be carried out via any of the accepted modes of administration of agents for serving similar utilities. The pharmaceutical compositions can be prepared by combining an immunomodulatory polypeptide or immunomodulatory polypeptide-containing composition or an antibody specific for PeptideX2-13 with an appropriate physiologically acceptable carrier, diluent or excipient, and may be formulated into preparations in solid, semisolid, liquid or microparticle-(e.g., microdroplet) containing gaseous forms, such as tablets, capsules, powders, granules, ointments, solutions, suppositories, injections, inhalants, gels, microspheres, and aerosols. In addition, other pharmaceutically active ingredients (including other immunosuppressive agents as described elsewhere herein) and/or suitable excipients such as salts, buffers and stabilizers may, but need not, be present within the composition. Administration may be achieved by a variety of different routes, including oral, parenteral, nasal, intravenous, intradermal, subcutaneous or topical.

In certain embodiments, the immunomodulatory polypeptides disclosed herein or antibodies specific therefor are administered via intravenous infusion for treating sepsis.

In certain other embodiments, the immunomodulatory polypeptides disclosed herein or antibodies specific therefor are administered topically (e.g., in form of eye drops or ointments) or via another route such as in form of oral pills, injections inside or around the eye, or intravenous infusions for treating uveitis.

In certain embodiments, the immunomodulatory polypeptides disclosed herein or antibodies specific therefor are administered topically or orally (e.g., in form of sublingual drops) or subcutaneous injections and/or intravenous infusion.

The immunomodulatory polypeptides prepared in solid state (e.g., in lyophilized form) may be solubilized in a physiologically acceptable buffer, such as Dulbecco's phosphate-buffered saline (DPBS) or a Hepes buffer. An exemplary DPBS buffer is a solution of 0.2% acetic acid, 0.4 M DPBS, and 1% to 2% DMSO, pH 6. Another exemplary DPBS buffer is a solution of 5% glycerol, 0.5 M DPBS, pH 7, which is preferred for dissolving the peptides as set forth in SEQ ID NOS: 5 (i.e., peptideX2 core) and 16 (i.e., reserve core). An exemplary Hepes buffer is a solution of 0.2% to 0.4% acetic acid, 0.4M Hepes, 0.4% glycerol, and 40 mM L-arginine, pH6, which is preferred for dissolving the peptide as set forth in (i.e., peptide X₂-13). The pH of the resulting solution may be in the range of pH 4 to 8, preferably at or lower than pH 7, more preferably at or lower than pH 6, such as at pH 5. The concentration of the immunomodulatory polypeptide is preferably below a concentration at which the immunomodulatory polypeptide starts to become insoluble (e.g., at or lower than 5 mg/ml, 4 mg/ml, or 3 mg/ml). It is also preferably that the immunomodulatory polypeptides or solutions thereof are used within a certain period of time after their preparation, such as within 60, 50, or 40 days, preferably within 30 days. Furthermore, preferably, any freeze and thaw cycle of the immunomodulatory polypeptide solution is to be avoided or minimized to maximize or maintain the solubility of the immunomodulatory polypeptide. The immunomodulatory polypeptide solution is preferably without or with a minimal amount of a compound or solvent that could be toxic to a subject to which the immunomodulatory polypeptide solution is to be administered.

Preferred modes of administration depend upon the nature of the condition to be treated or prevented, which in certain embodiments will refer to a deleterious or clinically undesirable condition the extent, severity, likelihood of occurrence and/or duration of which may be decreased (e.g., reduced in a statistically significant manner relative to an appropriate control situation such as an untreated control) according to certain methods provided herein. An amount that, following administration, detectably reduces, inhibits, prevents, decreases the severity or likelihood of occurrence of, or delays such a condition, for instance, the onset or exacerbation of rheumatoid arthritis (RA) or of sepsis, or the rejection of a transplant such as an organ allograft or bone marrow transplant, or the partial or complete reduction of a tumor burden, in a statistically significant manner is considered effective. Persons skilled in the relevant art will be familiar with any number of diagnostic, surgical and/or other clinical criteria that may indicate the clinical appropriateness of, and/or to which can be adapted, administration of the immunomodulatory compositions described herein. See, e.g., Littlejohn et al., 2018 Prim. Care 45 (2): 237-255; Wasserman, 2011 Am. Fam. Physician 84 (11): 1245-1252; Lee et al., 2001 Lancet 358 (92985): 903-911; Faix, 2013 Crit. Rev. Clin. Lab. Sci. 50 (1): 23-36 (“Biomarkers of Sepsis”); Wiersinga et al., 2014 Virulence 5 (1): 36-44 (“Host innate immune responses to sepsis”); Hotchkiss et al., 2013 Nat. Rev. Immunol. 13:862; Aziz et al., 2013 J. Leukoc. Biol. 93 (3): 329; Beyrau et al., 2012 Open Biol. 2:120134; Fry, 2012 Amer. Surg. 78:1; Kellum et al., 2007 Arch. Intern. Med. 167 (15): 1655; Remick, 2007 Am. J. Pathol. 170 (5): 1435; Hotchkiss et al., 2003 New Engl. J. Med. 348:138-150; Humar et al., Atlas of Organ Transplantation, 2006, Springer; Kuo et al., Comprehensive Atlas of Transplantation, 2004 Lippincott, Williams & Wilkins; Gruessner et al., Living Donor Organ Transplantation, 2007 McGraw-Hill Professional; Antin et al., Manual of Stem Cell and Bone Marrow Transplantation, 2009 Cambridge University Press; Wingard et al. (Ed.), Hematopoietic Stem Cell Transplantation: A Handbook for Clinicians, 2009 American Association of Blood Banks.

Neutrophil roles in inflammatory diseases and conditions such as sepsis, rheumatoid arthritis, and others, have been described and include an early dominant “hyperinflammatory phase” of potent TLR-mediated induction of inflammatory cytokine release (e.g., IL-6, TNFα, IL-1β) following PAMP recognition, which subsequently gives way to a concurrent but later-dominant “hypoinflammatory phase” of TLR-mediated immunosuppression following DAMP recognition, this latter phase characterized by apoptotic depletion of myeloid as well as lymphoid adaptive immune cells, release by neutrophils of the immunosuppressive cytokine IL-10, and reduced levels of inflammatory cytokines (e.g., Faix, 2013 Crit. Rev. Clin. Lab. Sci. 50 (1): 23-36; Wiersinga et al., 2014 Virulence 5 (1): 36-44; Fournier, 2013 Front. Cell. Infect. Microbiol. 2: Art. 167; Hotchkiss et al., 2013 Nat. Rev. Immunol. 13:862; Aziz et al., 2013 J. Leukoc. Biol. 93 (3): 329; Beyrau et al., 2012 Open Biol. 2:120134; Fry, 2012 Amer. Surg. 78:1; Kellum et al., 2007 Arch. Intern. Med. 167 (15): 1655; Remick, 2007 Am. J. Pathol. 170 (5): 1435; Hotchkiss et al., 2003 New Engl. J. Med. 348:138-150; Meng et al., 2004 J. Clin. Invest. 113 (10): 1473-1481; Decker, 2004 J. Clin. Invest. 113:1387-1389); Navarini et al., 2009 Proc. Nat. Acad. Sci. USA 106:7107-7112; Roger et al., 2009 Proc. Nat. Acad. Sci. USA 106:6889; Alves-Filho et al., 2009 Proc. Nat. Acad. Sci. USA 106:4018; Zou et al., 2011 Shock 36:370; Castoldi et al, 2012 PLOS ONE 7 (5): e37584; Pene et al., 2009 Infect. Immun. 77 (12): 5651).

The effects of an immunomodulatory polypeptide may vary as a function of dosage parameters (e.g., concentration, timing of administration, absence or presence of competing TLR2/4 ligands such as PAMPs or DAMPs, activation status of target cells, host clinical status, valency, etc.). The herein described PeptideX2-13 and its variants are thus believed according to non-limiting theory to provide useful immunomodulatory properties. The presently disclosed PeptideX2-13 and variants thereof afford such properties through their alteration (e.g., statistically significant increases or decreases) in the activity levels of one or more cellular regulators of immune status, which according to certain preferred embodiments and further according to non-limiting theory relate to unexpected advantages that are obtained by their dual functioning as (i) weak agonists of TLR2 and/or TLR4, through which biological signals are transduced that are qualitatively and quantitatively less profound than TLR2/TLR4 activation signals that are transduced in response to PAMPs or DAMPS, and (ii) antagonists of PAMPs and/or DAMPs by virtue of their competitive, albeit low affinity, binding to TLR2 and TLR4. In this respect, the presently disclosed PeptideX2-13 and certain variants thereof surprisingly permit the innate immune system to mediate a moderate inflammatory response instead of the exuberant hyperinflammatory reaction that characterizes sepsis, without driving the immune system to the immunosuppressed state that otherwise often subsequently predominates later stages of sepsis.

Preliminary animal and human trials may be performed to test the safety and efficacy of PeptideX2-13 [SEQ ID NO: 3], YLQMN [SEQ ID NO: 5] or NMQLY [SEQ ID NO: 16] for the treatment of TLR-mediated inflammatory processes such as those seen in RA and sepsis. For example and by way of non-limiting illustration, in a proposed human trial of PeptideX2-13 [SEQ ID NO: 3], YLQMN [SEQ ID NO: 5] or NMQLY [SEQ ID NO: 16] in sepsis, the immunomodulatory peptide is administered intravenously (e.g., via intravenous infusion) to achieve a serum concentration of about 50-100 μg/ml. Assuming a total body distribution volume of 6 liters, administration to a patient of 500 mg of PeptideX2-13 [SEQ ID NO: 3], YLQMN [SEQ ID NO: 5] or NMQLY [SEQ ID NO: 16] over 1 hour would represent an estimated plasma concentration of 80 μg/ml. As a reference for comparison, in the treatment of rheumatoid arthritis, the TNFα antagonist infliximab (Remicade®, Janssen Biotech Inc.) is typically administered intravenously to a patient weighing 70 kg at 3-10 mg/kg/dose, which would be calculated as delivery of 300 mg to 700 mg of infliximab as an infusion.

In another embodiment, the amount administered is sufficient to increase the rate of embryo implantation. In certain embodiments, the immunomodulatory polypeptide is administered to in vitro fertilization (IVF) patients (which may include a pseudopregnant patient such as a surrogate mother), and/or contacted with an IVF-generated embryo in vitro, to increase, promote or permit implantation of the embryo. In other embodiments, the immunomodulatory polypeptide is administered to an individual trying to get pregnant with or without prior diagnosed fertility difficulties. The effectiveness of an immunomodulatory polypeptide to modulate the immune response, and thereby modulate embryo implantation, can be determined using assays known in the art, such as, for example, the “PIF assays” described in U.S. Pat. Nos. 5,646,003 and 5,981,198 and PCT Application Publication Nos. WO 2003/004601 and WO 2005/040196, the disclosures of which are incorporated herein by reference in their entirety. Principles and practices in reproductive medicine are known to skilled clinicians, who will appreciate the factors involved in adaptation of the present disclosure to the clinical setting. See, e.g., Lebovic et al., Reproductive Endocrinology and Infertility: Handbook for Clinicians, 2005 Scrub Hill Press; Botros et al., Infertility and Assisted Reproduction, 2008 Cambridge Univ. Press; Greene et al., Creasy and Resnick's Maternal-Fetal Medicine: Principle and Practice, 2008 Saunders Publishing; Cunningham et al., Williams' Obstetrics-23rd Ed. 2009 McGraw-Hill Professional.

In other embodiments, the amount administered is sufficient to result in clinically relevant reduction in symptoms of preeclampsia, hemolysis-elevated liver enzymes-low platelet count (HELLP) syndrome and eclampsia, such as, but not limited to, reduction of any one or more of hypertension, edema of the hands and/or face, proteinuria, sudden weight gain, nausea, vomiting, abdominal pain, shoulder pain, lower back pain, muscle aches or pains, headache, changes in vision, blurry vision, hyperreflexia, racing pulse, mental confusion, anxiety, shortness of breath, hemolysis, elevated liver enzymes, low platelet count, decreased urine output, fatigue, fluid retention, nosebleeds, enlarged liver and seizures or convulsions.

In other embodiments, the amount of PeptideX2-13-containing polypeptide, a immunomodulatory polypeptide comprising or consisting of a variant of PeptideX2-13, or a specific anti-PeptideX2-13 antibody that is administered is sufficient to result in clinically relevant reduction in symptoms of inflammatory and/or autoimmune diseases, including but not limited to rheumatoid arthritis (RA), systemic lupus erythematosus (SLE), inflammatory bowel disease (IBD), psoriatic arthritis, Crohn's disease, ulcerative colitis, seronegative spondyloarthropathies, Behcet's disease, vasculitis, uveitis and other autoimmune diseases. Reduction in RA symptoms may be evidenced, for example by way of illustration and not limitation, as reduction of any one or more of fatigue, loss of appetite, low fever, swollen glands, weakness, swollen joints, joint pain, morning stiffness, warm, tender, and stiff joints when not used for as little as an hour, bilateral joint pain (fingers (but not the fingertips), wrists, elbows, shoulders, hips, knees, ankles, toes, jaw, and neck may be affected); loss of range of motion of affected joints, pleurisy, eye burning, eye itching, eye discharge, nodules under the skin, numbness, tingling, or burning in the hands and feet. Criteria for diagnosis and clinical monitoring of RA patients are well known to those skilled in the relevant art. See, e.g,. Hochberg et al., Rheumatology, 2010 Mosby; Firestein et al., Textbook of Rheumatology, 2008 Saunders. Criteria for diagnosis and clinical monitoring of patients having RA and/or other autoimmune diseases are also well known to those skilled in the relevant art. See, e.g., Petrov,

Autoimmune Disorders: Symptoms, Diagnosis and Treatment, 2011 Nova Biomedical Books; Mackay et al. (Eds.), The Autoimmune Diseases-Fourth Edition, 2006 Academic Press; Brenner (Ed.), Autoimmune Diseases: Symptoms, Diagnosis and Treatment, 2011 Nova Science Pub. Inc.

Certain embodiments contemplate a method of treating a malignant condition, comprising administering to a subject having or suspected of having a malignancy a composition that comprises a therapeutically effective amount of an immunomodulatory polypeptide that comprises or consists of either the PeptideX2-13 amino acid sequence set forth in SEQ ID NO: 3 or a variant thereof (e.g., SEQ ID NO: 4, 5, 10, 11, 12, 13, 14, 15, 16, 17 or 22) and thereby treating the malignant condition. According to certain such embodiments, the immunomodulatory polypeptide that comprises or consists of SEQ ID NO: 3, 4, 5, 10, 11, 12, 13, 14, 15, 16, 17 or 22 or the antibody that is capable of specifically binding to such a polypeptide promotes altered (e.g., increased or decreased in a statistically significant manner) immunological activity that results in immune system-potentiated killing of tumor cells and/or inhibition of tumor metastasis. By way of non-limiting theory, such altered immunological activity may be induced or promoted by binding of the herein described immunomodulatory polypeptide to one or more cognate receptors on immunologically active cells including neutrophils, or by interference with such binding by antibody blockade when an antibody (e.g., anti-PeptideX2-13 antibody) is administered.

Neutrophil roles in cancer cell rejection and metastasis have been described, including in breast cancer, ovarian cancer, adenoma, colorectal, gastric, lung, prostate and hepatocellular carcinoma, melanoma, and hematologic (e.g., leukemia, lymphoma) and other malignancies (e.g., DiCarlo et al., 2001 Blood 97;339; Mantovani et al., 2011 Nat. Rev. Immunol. 11:519; Gregory et al., 2011 Canc. Res. 71:2411; De Larco et al., 2004 Clin. Canc. Rec. 10:4895), as have anti-tumor effects of the known neutrophil products IL-6 and IL-10 (e.g., Li et al., 2010 Canc. Chemother. Pharmacol. 66:981; Mumm et al., 2011 Canc. Cell 20:781). The presently provided immunomodulatory polypeptides directly bind to neutrophils and after being contacted with neutrophil-containing peripheral blood leukocyte preparations, induce, inter alia, elaboration of IL-6 and IL-10. This effect on a local and/or systemic cytokine profile is thus believed to provide immunomodulatory properties of the herein described PeptideX2-13 polypeptides and/or of antibodies specific for such PeptideX2-13 polypeptides, through their alteration (e.g., statistically significant increases or decreases) in the activity levels of one or more cellular regulators of immune status. (See, e.g., DiCarlo et al., 2001 Blood 97;339; Mantovani et al., 2011 Nat. Rev. Immunol. 11:519.)

The presence of a malignant condition in a subject refers to the presence of dysplastic, cancerous and/or transformed cells in the subject, including, for example neoplastic, tumor, non-contact inhibited or oncogenically transformed cells, or the like (e.g., carcinomas such as adenocarcinoma, squamous cell carcinoma, small cell carcinoma, oat cell carcinoma, etc., sarcomas such as chondrosarcoma, osteosarcoma, etc.) which are known to the art and for which criteria for diagnosis and classification are established (e.g., Hanahan and Weinberg, 2011 Cell 144:646; Hanahan and Weinberg 2000 Cell 100:57; Cavallo et al., 2011 Canc. Immunol. Immunother. 60:319; Kyrigideis et al., 2010 J. Carcinog. 9:3). In preferred embodiments contemplated by the present disclosure, for example, such cancer cells may be cells of mixed lineage leukemia, esophageal cancer, ovarian cancer, prostate cancer, kidney cancer, colon cancer, liver cancer, stomach cancer, breast cancer and pancreatic cancer, and other solid cancers. The precise dosage and duration of treatment is a function of the condition or disease being treated and may be determined empirically using known testing protocols or by testing the compositions in model systems known in the art and extrapolating therefrom. Controlled clinical trials may also be performed. Dosages may also vary with the severity of the condition to be alleviated. A pharmaceutical composition is generally formulated and administered to exert a therapeutically useful effect while minimizing undesirable side effects. The composition may be administered one time or may be divided into a number of smaller doses to be administered at intervals of time. For any particular subject, specific dosage regimens may be adjusted over time according to the individual need.

The immunomodulatory-containing compositions may be administered alone or in combination with other known immunosuppressive treatments, such as monoclonal antibodies to lymphocytes and cytokine receptors (e.g., anti-IL-2Ra), calcineurin inhibitors (e.g., cyclosporine and tacrolimus), and cytokine receptor signal transduction inhibitors (e.g., sirolimus). The compositions may also be administered in combination with antibiotics.

Typical routes of administering these and related pharmaceutical compositions thus include, without limitation, oral, topical, transdermal, inhalation, parenteral, sublingual, buccal, rectal, vaginal, intranasal and ophthalimic. The term parenteral as used herein includes subcutaneous injections, intravenous, intramuscular, intrasternal injection or infusion techniques. Pharmaceutical compositions according to certain embodiments of the present disclosure are formulated to allow the active ingredients contained therein to be bioavailable upon administration of the composition to a patient. Compositions that will be administered to a subject or patient may take the form of one or more dosage units, where for example, a tablet may be a single dosage unit, and a container of a herein described immunomodulatory polypeptide in aerosol form may hold a plurality of dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition to be administered will, in any event, contain a therapeutically effective amount of an immunomodulatory polypeptide of the present disclosure, for treatment of a disease or condition of interest in accordance with teachings herein.

A pharmaceutical composition may be in the form of a solid or liquid. In one embodiment, the carrier(s) are particulate, so that the compositions are, for example, in tablet or powder form. The carrier(s) may be liquid, with the compositions being, for example, an oral oil, injectable liquid or an aerosol, which is useful in, for example, inhalatory administration. When intended for oral administration, the pharmaceutical composition is preferably in either solid or liquid form, where semisolid, semiliquid, suspension and gel forms are included within the forms considered herein as either solid or liquid.

As a solid composition for oral administration, the pharmaceutical composition may be formulated into a powder, granule, compressed tablet, pill, capsule, chewing gum, wafer or the like. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent. When the pharmaceutical composition is in the form of a capsule, for example, a gelatin capsule, it may contain, in addition to materials of the above type, a liquid carrier such as polyethylene glycol or oil.

The pharmaceutical composition may be in the form of a liquid, for example, an elixir, syrup, solution, emulsion or suspension. The liquid may be for oral administration or for delivery by injection, as two examples. When intended for oral administration, preferred composition contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

The liquid pharmaceutical compositions, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A liquid pharmaceutical composition intended for either parenteral or oral administration should contain an amount of an immunomodulatory polypeptide as herein disclosed such that a suitable dosage will be obtained. Typically, this amount is at least 0.01% of the immunomodulatory polypeptide in the composition. When intended for oral administration, this amount may be varied to be between 0.1 and about 70% of the weight of the composition. Certain oral pharmaceutical compositions contain between about 4% and about 75% of the immunomodulatory polypeptide. In certain embodiments, pharmaceutical compositions and preparations according to the present disclosure are prepared so that a parenteral dosage unit contains between 0.01 to 10% by weight of the immunomodulatory polypeptide prior to dilution.

The pharmaceutical composition may be intended for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a pharmaceutical composition for topical administration. If intended for transdermal administration, the composition may include a transdermal patch or iontophoresis device. The pharmaceutical composition may be intended for rectal administration, in the form, for example, of a suppository, which will melt in the rectum and release the drug. The composition for rectal administration may contain an oleaginous base as a suitable nonirritating excipient. Such bases include, without limitation, lanolin, cocoa butter and polyethylene glycol.

The pharmaceutical composition may be intended for ophthalmic application. Preferably, solutions are prepared using a physiological saline solution as a major vehicle. The pH of such ophthalmic solutions should preferably be maintained between 4.5 and 8.0 with an appropriate buffer system. The formulations may also contain conventional pharmaceutically acceptable preservatives, stabilizers and/or surfactants. The ophthalmic formulations may be conveniently packaged in forms suitable for metered application, such as in containers equipped with a dropper, to facilitate application to the eye. One package may contain one or more unit doses. Preservative-free solutions are often formulated in non-resealable containers containing up to about ten units doses where a typical unit dose is from one to about 8 drops, such as one to about 3 drops.

The pharmaceutical composition may include various materials, which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule. The pharmaceutical composition in solid or liquid form may include an agent that binds to the immunomodulatory polypeptide of the present disclosure and thereby assists in the delivery of the compound. Suitable agents that may act in this capacity include monoclonal or polyclonal antibodies, one or more proteins or a liposome. The pharmaceutical composition may consist essentially of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols may be delivered in single phase, biphasic, or triphasic systems to deliver the active ingredient(s). Delivery of the aerosol includes the necessary container, activators, valves, subcontainers, and the like, which together may form a kit. One of ordinary skill in the art, without undue experimentation may determine preferred aerosols.

The pharmaceutical compositions may be prepared by methodology well known in the pharmaceutical art. For example, a pharmaceutical composition intended to be administered by injection can be prepared by combining a composition that comprises an immunomodulatory polypeptide as described herein and optionally, one or more of salts, buffers and/or stabilizers, with sterile, distilled water to form a solution. A surfactant may be added to facilitate the formation of a homogeneous solution or suspension. Surfactants are compounds that noncovalently interact with the peptide composition to facilitate dissolution or homogeneous suspension of the immunomodulatory polypeptide in the aqueous delivery system.

The compositions are administered in a therapeutically effective amount, which will vary depending upon a variety of factors including the activity of the specific compound (e.g., PeptideX2-13 [SEQ ID NO: 3], YLQMN [SEQ ID NO:5] and NMQLY [SEQ ID NO: 16]) employed; the metabolic stability and length of action of the compound; the age, body weight, general health, sex, and diet of the patient; the mode and time of administration; the rate of excretion; the drug combination; the severity of the particular disorder or condition; and the subject undergoing therapy. Generally, a therapeutically effective daily dose is (for a 70 kg mammal) from about 0.001 mg/kg (i.e., 0.07 mg) to about 100 mg/kg (i.e., 7.0 g); preferably a therapeutically effective dose is (for a 70 kg mammal) from about 0.01 mg/kg (i.e., 0.7 mg) to about 50 mg/kg (i.e., 3.5 g); more preferably a therapeutically effective dose is (for a 70 kg mammal) from about 1 mg/kg (i.e., 70 mg) to about 25 mg/kg (i.e., 1.75 g).

Compositions comprising the immunomodulatory polypeptides of the present disclosure may also be administered simultaneously with, prior to, or after administration of one or more other therapeutic agents. Such combination therapy may include administration of a single pharmaceutical dosage formulation which contains an immunomodulatory polypeptide and one or more additional active agents, as well as administration of compositions comprising antibodies of the present disclosure and each active agent in its own separate pharmaceutical dosage formulation. For example, an immunomodulatory polypeptide as described herein and the other active agent can be administered to the patient together in a single oral dosage composition such as a tablet or capsule, or each agent administered in separate oral dosage formulations. Similarly, an immunomodulatory polypeptide as described herein and the other active agent can be administered to the patient together in a single parenteral dosage composition such as in a saline solution or other physiologically acceptable solution, or each agent administered in separate parenteral dosage formulations. Where separate dosage formulations are used, the compositions comprising antibodies and one or more additional active agents can be administered at essentially the same time, i.e., concurrently, or at separately staggered times, i.e., sequentially and in any order; combination therapy is understood to include all these regimens.

Thus, in certain embodiments, also contemplated is the administration of immunomodulatory polypeptide compositions of this disclosure in combination with one or more other therapeutic agents. Such therapeutic agents may be accepted in the art as a standard treatment for a particular disease state as described herein, such as rheumatoid arthritis, inflammation or preeclampsia. Exemplary therapeutic agents contemplated include cytokines, growth factors, steroids, NSAIDs, DMARDs, anti-inflammatoires, chemotherapeutics, or other active and ancillary agents.

In various embodiments, the immunomodulatory polypeptides described herein are conjugated to a detectable label that may be detected directly or indirectly. In this regard, an immunomodulatory polypeptide “conjugate” refers to an immunomodulatory polypeptide that is covalently linked to a detectable label. In the present disclosure, DNA probes, RNA probes, monoclonal antibodies, antigen-binding fragments thereof, and antibody derivatives thereof, such as a single-chain-variable-fragment antibody or an epitope tagged antibody, may all be covalently linked to a detectable label. In “direct detection”, only one detectable antibody is used, i.e., a primary detectable antibody. Thus, direct detection means that the antibody that is conjugated to a detectable label may be detected, per se, without the need for the addition of a second antibody (secondary antibody).

A “detectable label” is a molecule or material that can produce a detectable (such as visually, electronically or otherwise) signal that indicates the presence and/or concentration of the label in a sample. When conjugated to a peptide, the detectable label can be used to locate and/or quantify the target to which the specific peptide is bound. Thereby, the presence and/or concentration of the target in a sample can be detected by detecting the signal produced by the detectable label. A detectable label can be detected directly or indirectly, and several different detectable labels conjugated to different specific antibodies can be used in combination to detect one or more targets.

Examples of detectable labels, which may be detected directly, include fluorescent dyes and radioactive substances and metal particles. In contrast, indirect detection requires the application of one or more additional antibodies, i.e., secondary antibodies, after application of the primary antibody. Thus, the detection is performed by the detection of the binding of the secondary antibody or binding agent to the primary detectable antibody. Examples of primary detectable binding agents or antibodies requiring addition of a secondary binding agent or antibody include enzymatic detectable binding agents and hapten detectable binding agents or antibodies.

In some embodiments, the detectable label is conjugated to a nucleic acid polymer which comprises the first binding agent (e.g., in an ISH, WISH, or FISH process). In other embodiments, the detectable label is conjugated to an antibody which comprises the first binding agent (e.g., in an IHC process).

Examples of detectable labels which may be conjugated to immunomodulatory polypeptides used in the methods of the present disclosure include fluorescent labels, enzyme labels, radioisotopes, chemiluminescent labels, electrochemiluminescent labels, bioluminescent labels, polymers, polymer particles, metal particles, haptens, and dyes.

Examples of fluorescent labels include 5-(and 6)-carboxyfluorescein, 5- or 6-carboxyfluorescein, 6-(fluorescein)-5-(and 6)-carboxamido hexanoic acid, fluorescein isothiocyanate, rhodamine, tetramethylrhodamine, and dyes such as Cy2, Cy3, and Cy5, optionally substituted coumarin including AMCA, PerCP, phycobiliproteins including R-phycoerythrin (RPE) and allophycoerythrin (APC), Texas Red, Princeton Red, green fluorescent protein (GFP) and analogues thereof, and conjugates of R-phycoerythrin or allophycoerythrin, inorganic fluorescent labels such as particles based on semiconductor material like coated CdSe nanocrystallites.

Examples of polymer particle labels include micro particles or latex particles of polystyrene, PMMA or silica, which can be embedded with fluorescent dyes, or polymer micelles or capsules which contain dyes, enzymes or substrates.

Examples of metal particle labels include gold particles and coated gold particles, which can be converted by silver stains. Examples of haptens include DNP, fluorescein isothiocyanate (FITC), biotin, and digoxigenin. Examples of enzymatic labels include horseradish peroxidase (HRP), alkaline phosphatase (ALP or AP), β-galactosidase (GAL), glucose-6-phosphate dehydrogenase, β-N-acetylglucosamimidase, β-glucuronidase, invertase, Xanthine Oxidase, firefly luciferase and glucose oxidase (GO). Examples of commonly used substrates for horseradishperoxidase include 3,3′-diaminobenzidine (DAB), diaminobenzidine with nickel enhancement, 3-amino-9-ethylcarbazole (AEC), Benzidine dihydrochloride (BDHC), Hanker-Yates reagent (HYR), Indophane blue (IB), tetramethylbenzidine (TMB), 4-chloro-1-naphtol (CN),.alpha.-naphtol pyronin (.alpha.-NP), o-dianisidine (OD), 5-bromo-4-chloro-3-indolylphosp-hate (BCIP), Nitro blue tetrazolium (NBT), 2-(p-iodophenyl)-3-p-nitropheny-1-5-phenyl tetrazolium chloride (INT), tetranitro blue tetrazolium (TNBT), 5-bromo-4-chloro-3-indoxyl-beta-D-galactoside/ferro-ferricyanide (BCIG/FF).

Examples of commonly used substrates for Alkaline Phosphatase include Naphthol-AS-B 1-phosphate/fast red TR (NABP/FR), Naphthol-AS-MX-phosphate/fast red TR (NAMP/FR), Naphthol-AS-B1-phosphate/-fast red TR (NABP/FR), Naphthol-AS-MX-phosphate/fast red TR (NAMP/FR), Naphthol-AS-B1-phosphate/new fuschin (NABP/NF), bromochloroindolyl phosphate/nitroblue tetrazolium (BCIP/NBT), 5-Bromo-4-chloro-3-indolyl-b-d-galactopyranoside (BCIG).

Examples of luminescent labels include luminol, isoluminol, acridinium esters, 1,2-dioxetanes and pyridopyridazines. Examples of electrochemiluminescent labels include ruthenium derivatives. Examples of radioactive labels include radioactive isotopes of iodide, cobalt, selenium, tritium, carbon, sulfur and phosphorous.

Detectable labels may be linked to the immunomodulatory polypeptides described herein or to any other molecule that specifically binds to a biological marker of interest, e.g., an antibody, a nucleic acid probe, or a polymer. Furthermore, one of ordinary skill in the art would appreciate that detectable labels can also be conjugated to second, and/or third, and/or fourth, and/or fifth binding agents or antibodies, etc. Moreover, the skilled artisan would appreciate that each additional binding agent or antibody used to characterize a biological marker of interest may serve as a signal amplification step. The biological marker may be detected visually using, e.g., light microscopy, fluorescent microscopy, electron microscopy where the detectable substance is for example a dye, a colloidal gold particle, a luminescent reagent. Visually detectable substances bound to a biological marker may also be detected using a spectrophotometer. Where the detectable substance is a radioactive isotope detection can be visually by autoradiography, or non-visually using a scintillation counter. See, e.g., Larsson, 1988, Immunocytochemistry: Theory and Practice, (CRC Press, Boca Raton, Fla.); Methods in Molecular Biology, vol. 80 1998, John D. Pound (ed.) (Humana Press, Totowa, N.J.).

The present disclosure further provides kits for detecting immunomodulatory polypeptides disclosed herein or cells (e.g., neutrophils) in a sample, wherein the kits contain at least one antibody, polypeptide, polynucleotide, vector or host cell as described herein. In certain embodiments, a kit may comprise buffers, enzymes, labels, substrates, beads or other surfaces to which the antibodies of the present disclosure are attached, and the like, and instructions for use.

As used in this specification and the appended claims, the singular forms “a,” “an” and “the” include plural references unless the content clearly dictates otherwise. Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers. Each embodiment in this specification is to be applied mutatis mutandis to every other embodiment unless expressly stated otherwise.

### EXAMPLES

### Example 1

### Peptidex2-13 (Seq Id No: 3) is a Ylqmn (Seq Id No: 5)-Containing Immunomodulatory Polypeptide

Structural and functional properties of the pentide YLQMN (SEQ ID NO: 5)-containing immunomodulatory polypeptide known as PeptideX2 (SEQ ID NO: 1) have previously been disclosed in U.S. Pat. Nos. 10,365,275; 9,816,989; 10,871,489; and WO 2014/165282. Briefly, PeptideX2 (KSIAYLQMNSLK, SEQ ID NO: 1) was shown, inter alia, to modestly induce human peripheral white blood cells to elaborate IL-6, IL-10 and TNFα when exposed to cells as a free polypeptide and to more potently induce these cytokines when presented to cells in multivalent form on a carrier molecule; to activate biological signal transduction by binding interactions with TLR2 and TLR4; and to selectively bind to a subset of human neutrophils. The present Example describes TLR-engaging activity of the pentide YLQMN [SEQ ID NO: 5]-containing immunomodulatory polypeptide G2-PeptideX2 (GKSIAYLQMNSLKG, SEQ ID NO: 2), and immunomodulatory activity of the pentide YLQMN [SEQ ID NO: 5]-containing immunomodulatory polypeptides PeptideX2 (SEQ ID NO: 1) and PeptideX2-13 (GKAAYLQMNAAKG, SEQ ID NO: 3) in an animal model of rheumatoid arthritis.

A. Collagen-Induced Lewis Rat Model of Rheumatoid Arthritis (RA)-PeptideX2 (SEQ ID NO: 1). The Lewis rat system is an art-accepted in vivo animal model of collagen-induced rheumatoid arthritis. Lewis rats obtained from a commercial laboratory supply vendor were maintained during a quarantine period under standard animal care conditions and found to be free of any clinical signs of disease or distress. Bovine type II collagen (10 mg, Chondrex, Inc., Redmond, WA) was dissolved in 2.5 ml 0.1M acetic acid and stirred overnight at 4-8° C. in the dark, and then one ml of this collagen solution was emulsified in one ml of incomplete Freund's adjuvant (ICFA, Sigma, St. Louis, MO) on an ice bath. On day zero, each of five rats was injected with 0.2 ml of collagen/ICFA emulsion at two sites and then observed for the next 30 days with the arthritis index (AI) calculated daily using the 16-point scoring method of Bevaart et al. (2010 Meths. Mol. Biol. 602:181: 0 points, no joint swelling or edema; 1 point, slight edema and erythema limited to foot and/or ankle; 2 points, edema and erythema in the toes and most ankle joints; 3 points, severe edema and erythema in the paw below the ankle joint; 4 points, edema and erythema of all four paws including the ankle joint).

On day 15, the rats were divided into treatment groups and were dosed intravenously on a daily basis for the next 15 days (except as noted) with the pentide YLQMN (SEQ ID NO: 5)-containing PeptideX2 (SEQ ID NO: 1) to achieve final circulating concentrations of PeptideX2 peptide as follows: 0 μg/ml (vehicle control: 0.2% acetic acid-Dulbecco's phosphate buffered saline (PBS-D), pH 6.0); 100 μg/ml vehicle; 200 μg/ml vehicle, and 500 μg/ml vehicle per rat/per day. As a positive control for anti-inflammatory activity, daily dosing of a control rat with prednisolone at 10 mg/kg/day was used. The arthritis index was calculated daily for each viable rat throughout the 30-day study.

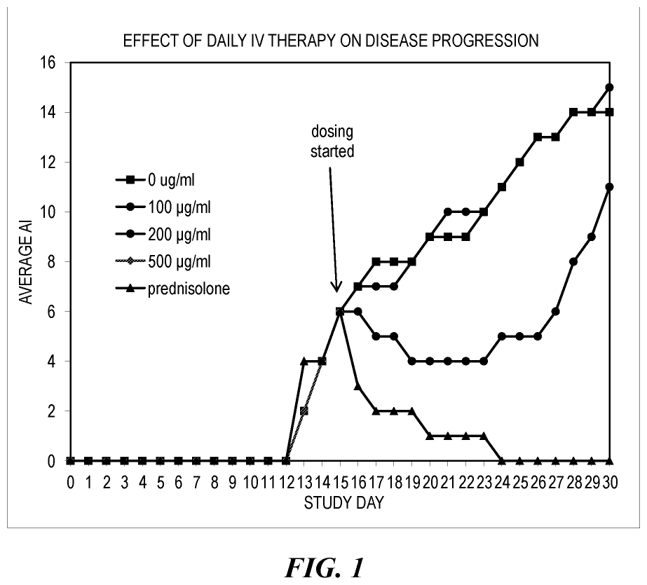

Results: The positive control prednisolone treatment of the Lewis rat in the collagen-induced rheumatoid arthritis model resulted in potent and sustained reduction of the arthritis index (FIG. 1, solid triangles).

The rats receiving PeptideX2 peptide at final concentrations of 0 μg/ml (vehicle control) and 100 μg/ml showed no significant difference in arthritis index (FIG. 1, solid squares, solid circles). The rat receiving the highest dose of PeptideX2 (500 μg/ml) went into respiratory distress and died immediately after receiving intravenous PeptideX2.

For over 10 days of daily treatment (which started on study day 15), administration of PeptideX2 at a final concentration of 200 μg/ml (FIG. 1, open circles with dashed line) resulted in a significant (60%) decrease in the arthritis index, relative to the vehicle control (0 ug/ml, see FIG. 1). In this in vivo study of the effects of the pentide YLQMN (SEQ ID NO: 5)-containing PeptideX2 (SEQ ID NO: 1) in collagen-induced arthritis in Lewis rats as an animal model of rheumatoid arthritis, administration of PeptideX2 (SEQ ID NO: 1) decreased the arthritis index of the experimental animals.

B. Stability, aggregation, and toxicity of the pentide YLQMN (SEQ ID NO: 5)-containing polypeptides PeptideX2 (SEQ ID NO: 1) and G2-PeptideX2 (SEQ ID NO: 2). In studies designed to prepare PeptideX2 in higher concentrations that would be tolerated in the test animals without inducing terminal respiratory distress, it was observed that at pH>6 or concentrations>4 mg/ml, PeptideX2 (SEQ ID NO: 1) solutions became turbid and exhibited flocculence, behavior that was exacerbated by freeze-thawing (data not shown). Even at pH 5, however, PeptideX2 dosages above 200 μg/ml again induced rapid terminal respiratory distress in the Lewis rat RA model, accompanied by convulsions and internal hemorrhage in liver, kidneys, bladder, and lungs, apparently a response to PeptideX2 aggregates that formed at these concentrations and consistent with strong TLR2 agonist activity exhibited by multivalent PeptideX2 when borne on a keyhole limpet hemocyanin carrier (e.g., U.S. Pat. No. 10,365,275, FIG. 10).

Modification of the PeptideX2 structure (KSIAYLQMNSLK, SEQ ID NO: 1) was therefore undertaken to obtain artificial pentide (YLQMN, SEQ ID NO: 5)-containing peptides that remained in solution without aggregating at pH around 6.0. Pentide (YLQMN, SEQ ID NO: 5) and a modified PeptideX designated G2-PeptideX2 (GKSIAYLQMNSLKG, SEQ ID NO: 2) did not aggregate in solutions of pH around 6.0 and accordingly were tested for their ability to transduce a biological activation signal by engaging cell surface TLR2.

HEK293 cells (50,000 to 75,000 cells/well) expressing a transfected human TLR2 gene (InvivoGen, San Diego, CA) were plated in wells of a 96-well plate (200 μl total volume) containing either G2-PeptideX2 (SEQ ID NO: 2) (in 20 μl buffer), pentide (YLQMN, SEQ ID NO: 5), or buffer only (20 μl) as a vehicle control. All cells contained a reporter construct having coding sequence for Secreted Embryonic Alkaline Phosphatase (SEAP) under the control of a promoter inducible by the transcription of NF-κB, and were incubated in media containing a detectable SEAP expression indicator (InvivoGen, San Diego, CA). After 16-20 hours incubation at 37° C., the optical density of culture supernatants was read at 650 nm on a Molecular

Devices SpectraMax™ 340PC absorbance detector.

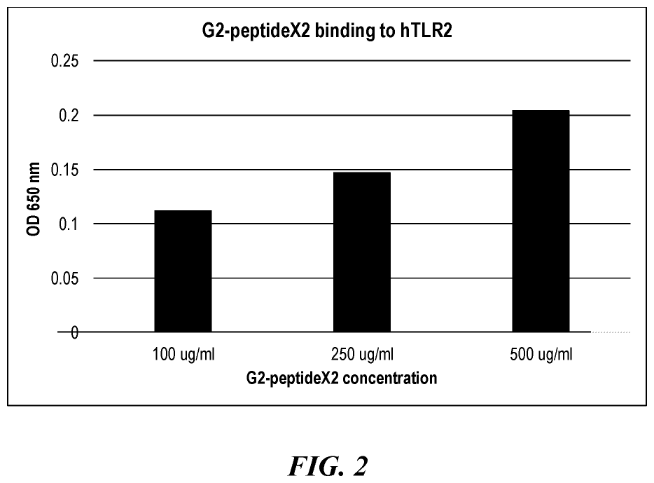

FIG. 2 shows the effects of soluble, non-self-aggregating G2-peptideX2 (GKSIAYLQMNSLKG, SEQ ID NO: 2) at concentrations of 100 μg/ml, 250 μg/ml, and 500 μg/ml on TLR2-driven NF-κB activation in HEK293 cell lines transfected with human TLR2. Levels of TLR2-driven NF-κB activation similar to those shown in FIG. 2 for G2-peptideX2 were also observed in response to peptideX2 (KSIAYLQMNSLK, SEQ ID NO: 1) and in response to the peptideX2-derived pentameric peptide referred to herein as “pentide” (YLQMN, SEQ ID NO: 5) (data not shown).

### Example 2

### Efficacy in the Collagen-Induced Lewis Rat Model of Rheumatoid Arthritis (Ra) of Another Pentide-Containing Immunomodulatory Polypeptide, Peptidex2-13 (Gkaaylqmnaakg, Seq Id No: 3).

PeptideX2-13 (SEQ ID NO: 3) is another solution-stable, non-aggregating, pentide-containing immunomodulatory polypeptide that did not produce flocculence at concentrations above 200 μM in solutions of approximately pH 6 and did not otherwise exhibit signs of self-aggregation. PeptideX2-13 was tested in the collagen-induced Lewis rat model of RA essentially as described above in Example 1 except that animals were treated with PeptideX2-13 (SEQ ID NO: 3) instead of with PeptideX2 (SEQ ID NO: 1).

Eleven Lewis rats were prepared using the collagen induced arthritis protocol. At day 15 thru day 30, three rats were given daily dosing of PeptideX2-13 (SEQ ID NO: 3) at 100 μg/ml, three rats were given daily dosing of PeptideX2-13 (SEQ ID NO: 3) at 250 μg/ml, and three rats were given daily dosing of PeptideX2-13 (SEQ ID NO: 3) at 500 μg/ml; as a positive control group, two rats were given prednisolone at 10 mg/kg/day. No adverse effects were observed in any of the animals.

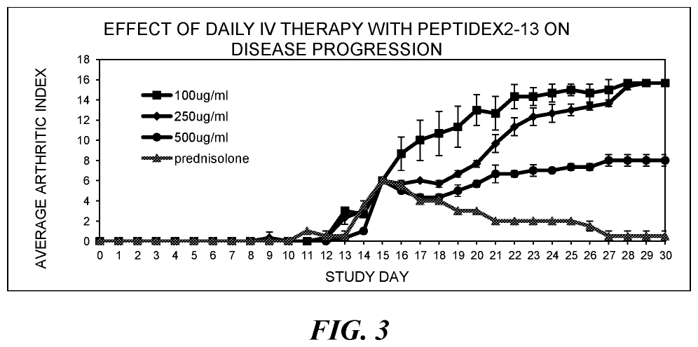

FIG. 3 shows results from the in vivo study of the effects of the non-self-aggregating PeptideX2-13 (SEQ ID NO: 3) in the Lewis rat collagen-induced arthritis RA model. Administration of PeptideX2-13 (SEQ ID NO: 3) at 250 μg/ml (solid diamonds) and 500 μg/ml (solid circles) decreased the arthritis index in the experimental animals in a dosage-dependent manner, while the lowest dosage of 100 μg/ml (open squares) had minimal effect. Average arthritic index was also decreased by the positive control anti-inflammatory corticosteroid compound, prednisolone (solid triangles).

### Example 3

Peptidex2-13 Interaction with Human Tlr2

This Example describes human TLR2 (hTLR2) agonist activity of PeptideX2-13 (SEQ ID NO: 3) and K2R/K12R-substituted PeptideX2-13 (GRAAYLQMNAARG, SEQ ID NO: 10). A modified variant of the above-described non-self-aggregating PeptideX2-13 (SEQ ID NO: 3) was produced in which the lysine (K) residues at positions 2 and 12 were replaced with arginine (R) residues. The resulting product was another solution-stable, non-aggregating, pentide (SEQ ID NO: 5)-containing tridecameric immunomodulatory polypeptide that did not produce flocculence at concentrations above 200 μM in solutions of approximately pH 6 and did not otherwise exhibit signs of self-aggregation. The K2R/K12R-substituted PeptideX2-13

GRAAYLQMNAARG (SEQ ID NO: 10) was a species within the genus of immunomodulatory polypeptides which comprises the amino acid sequence of general formula G-X₁-AAYLQMNAA-X₂-G as set forth in SEQ ID NO: 4 wherein X₁ and X₂ are independently selected from K and R.

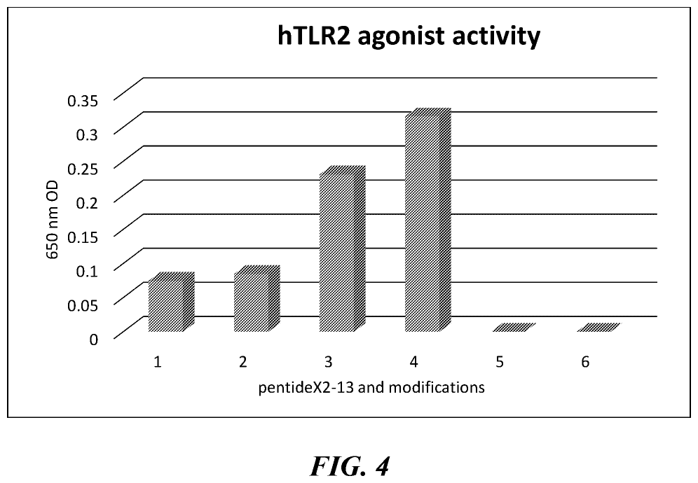

PeptideX2-13 (SEQ ID NO: 3) and K2R/K12R-substituted PeptideX2-13 (SEQ ID NO: 10) were tested for their ability to transduce a biological activation signal by engaging cell surface TLR2. HEK293 cells (50,000 to 75,000 cells/well) expressing a transfected human TLR2 gene (InvivoGen, San Diego, CA) were plated in wells of a 96-well plate (200 μl total volume) containing the indicated test peptide in 20 μl buffer. Test peptides were either soluble PeptideX2-13 (SEQ ID NO: 3) (FIG. 4, lane 1), soluble K2R/K12R-substituted PeptideX2-13 (SEQ ID NO: 10) (FIG. 4, lane 2), insoluble aggregates of modified PeptideX2-13 in which amino acids at positions 3, 4, 10 and 11 were substituted with nonpolar amino acids (FIG. 4, lanes 3-4), or soluble variants of PeptideX2-13 in which glycine at positions 1 and 13 was replaced with proline (FIG. 4, lane 5) or in which the amino acids at positions 3, 4, 10, and 11 were deleted (FIG. 4, lane 6). All cells contained a reporter construct having coding sequence for secreted embryonic alkaline phosphatase (SEAP) under the control of a promoter inducible by the transcription of NF-κB, and were incubated in media containing a detectable SEAP expression indicator (InvivoGen, San Diego, CA). After 16-20 hours incubation at 37° C., the optical density of culture supernatants was read at 650 nm on a Molecular Devices SpectraMax™ 340PC absorbance detector.

FIG. 4 shows the effects of soluble, non-self-aggregating PeptideX2-13 (SEQ ID NO: 3, lane 1) and soluble, non-self aggregating K2R/K12R-substituted PeptideX2-13 (SEQ ID NO: 10, lane 2) on TLR2-driven NF-κB activation in HEK293 cell lines transfected with human TLR2. Higher levels of TLR2-driven NF-κB were observed in response to the insoluble aggregates of alternatively modified peptide sequences (SEQ ID NOS: 20 and 21) (lanes 3-4) while the other soluble alternatively modified peptide sequences did not elicit a detectable response (SEQ ID NOS: 12 and 22) (lanes 5-6).

### Example 4

In Vitro Allograft Cell Proliferation Assays with Peptidex2-13

A modified mixed lymphocyte reaction (MMLR) is used to determine if the immunomodulating properties of PeptideX2-13 (SEQ ID NO: 3) are sufficient to suppress cell proliferation in an in vitro allograft model. Mixed lymphocyte reactions are used to test the compatibility of lymphocytes from two individuals. One set of lymphocytes is irradiated or treated with mitomycin C so that they cannot respond or proliferate in response to a stimulus, and the other set of lymphocytes are responder cells which can differentiate into effector cells and proliferate if they are alloreactive to the first set of lymphocytes. The MMLR includes test groups featuring the addition of PeptideX2-13 to determine its suppressive effect on cell proliferation.

Cultures of peripheral white blood cells of eight human subjects obtained with appropriate informed consent are analyzed using one-way and two-way MMLR. Cells are cultured with 250 μg/ml PeptideX2-13 in one-way reactions using mitomicyin C. Two-way MMLR are also performed using 250 μg/ml PeptideX2-13. Cell proliferation is measured using a BrdU colorimetric assay according to the manufacturer's instructions (Roche Applied Science Product #11647229001). Inhibition of cellular proliferation by at least 50% indicates that a concentration of PeptideX2-13 is sufficient to suppress cell proliferation.

Optimum dosing of PeptideX2-13 is determined by measuring the degree of inhibition of cellular proliferation using various concentrations of PeptideX2-13. The concentrations include 25, 50, 100, 250 and 500 μg/ml. The dose of PeptideX2-13 that results in the greatest alteration (e.g., statistically significant decrease in cytoproliferation) in MMLR assays is regarded as the optimum dose for suppressing cellular proliferation.

### Example 5

In Vitro Xenograft Cell Proliferation Assays with Peptidex2-13

Similarly, MMLR assays are used to determine if PeptideX2-13 may be useful in suppressing cellular proliferation as an in vitro xenograft model. The MMLR assays are performed as described above, however, mixtures of human and non-human leukocytes are used to determine the optimum dose or concentration of PeptideX2-13 for suppressing cellular proliferation in a xenograft model. The non-human leukocytes are obtained from pigs, rats and monkeys.

### Example 6

### Generation of Peptidex2-13 Variants

Variants of PeptideX2-13 are generated by introducing amino acid substitutions at different positions in the PeptideX2-13 amino acid sequence in order to determine if such variations enhance the biological activity of PeptideX2-13. Preferred variants are polypeptides of 12, 13, 14, 15, 16, 17, 18, 19, 20, 21, 22, 23, 24, 25, 26, 27, 28, 29, 30 or 31 amino acids that comprise at least one amino acid sequence as set forth in SEQ ID NO: 4, for example the K2R/K12R-substituted PeptideX2-13 polypeptide having the amino acid sequence set forth in SEQ ID NO: 10; in embodiments where the peptide comprises a polypeptide of SEQ ID NO: 4 that differs in amino acid sequence from SEQ ID NO: 3, polypeptides of no more than 13, 14, 15, 16, 17, 18, 19, 20, 21, 22, 23, 24, 25, 26, 27, 28, 29, 30 or 31 amino acids, or of no more than 32, 33, 34, 35, 36, 37, 38, 39, 40, 41, 42, 43, 44, 45, 46, 47, 48, 49, 50, 51, 52, 53, 54, 55, 56, 57, 58, 59, 60, 61-70, 71-80 or 81-90 amino acids may be used. The modified peptides are tested as candidate competitive inhibitors of PeptideX2-13-FITC (described above) binding to neutrophils, using flow cytometry in order to compare their activity. Competitive cell-binding assays are performed by flow cytometry as described above, and cytokine induction profiles are also generated as described above, in order to compare the modified PeptideX2-13 sequences with unmodified PeptideX2-13. Peptides exhibiting significant activity are selected for use in MMLR and in vivo models of sepsis, RA, atherosclerosis, and/or transplant models having relevance to organ transplantation and allograft rejection, as discussed herein.

In flow cytometry-based competitive binding assays, cells are incubated with either PeptideX2-13-FITC or PeptideX2-13-FITC conjugated to BSA and the PeptideX2-13 sequence variant, to determine if the variant competes with PeptideX2-13. The competitive binding assays are used in addition to the cytokine profile results in order to determine candidate PeptideX2-13 variants for use in medical treatments such as treatment of sepsis, RA, atherosclerosis, and/or for transplantation.

### Example 7

### In Vivo Transplant Model

In order to further examine the potential role of PeptideX2-13 (SEQ ID NO: 3), or a suitable variant (e.g., SEQ ID NO: 4, SEQ ID NO: 10, or other variant), established in vivo allograft and xenograft models are utilized. For a xenograft model, human tissue is transplanted into an animal, such as a rodent or primate. Control animals are not treated with PeptideX2-13, and test animals are treated with either PeptideX2-13 or a standard immunosuppressive agent, such as anti-IL-2Ra. The rate of graft acceptance and/or rejection between the treatment groups is compared.

### Example 8

### Immune Status Profile of Peptidex2-13-Induced Immunomodulation

Immune status profiling is performed by determining the effects, on peripheral blood white blood cells, of exposure to a herein provided polypeptide that comprises the PeptideX2-13 sequence (SEQ ID NO: 3) or that comprises a PeptideX2-13 variant sequence (e.g., SEQ ID NOS: 4 or 10) as described herein. MLR and MMLR assays are performed as described above, and the effects of PeptideX2-13 (SEQ ID NO: 3) sequence-containing polypeptides or a PeptideX2-13 variant (e.g., SEQ ID NOS: 4 or 10) sequence-containing polypeptides on MLR and MMLR are determined by measuring cellular proliferation and also by characterizing supernatant fluids for released cytokines using art accepted assay methodologies (e.g., Ready-Set-Go™ ELISA kit from eBiosciences, San Diego, CA; or with immunoassay kits as are readily available from R & D Systems, Minneapolis, MN, or from BD Biosciences, San Jose, CA; or using other well-known methodologies for detecting and quantifying cytokines). The effects of PeptideX13 or Peptide132 variant sequence-containing polypeptides are also assessed on in vitro peripheral blood leukocyte (PBL) cultures following stimulation with T cell mitogens (e.g., PHA, or costimulatory antibody combinations such as anti-CD3/anti-CD28, etc.) or with B cell mitogens (e.g., LPS, or costimulatory antibody combinations, etc.) or with other established PBL subpopulation-specific or non-specific stimulatory protocols.

Following in vitro stimulation in controlled experimental groups that include such treatments in the presence and absence of a PeptideX13 or PeptideX13 variant sequence-containing polypeptide as provided herein, culture fluids are separated from cells and tested for the presence of one or more members of a panel of immunologically relevant cytokines. The panel includes: IL-2, IL-4, IL-6, IL-8, IL-10, IL-12, and TNF-α (detection of these cytokines is described, for example, in U.S. Pat. Nos. 10,365,275; 9,816,989; 10,871,489; and WO 2014/165282) and also includes one or more of IL-1β, IL-5, IL-8, IL-13, IL-17, IL-22, CCL2, CCL3, CCL4, CCL5, CCL11, CXCL5, CXCL11, TGFβ, IFNγ, basic FGF, GCSF, GMCSF, VEGF, EGF, and HGF.

Peripheral blood white cells containing peripheral blood leukocytes are also sorted using fluorescence activated cell sorting (FACS) on the basis of positive staining with labeled PeptideX2-13, and the positively selected cells are cultured in culture medium alone or in medium supplemented with PeptideX2-13 and/or other neutrophil-inducing agents. At suitable timepoints medium aliquots are collected and separated from cells, and the supernatant fluids are tested for the presence of one or more of the members of the panel of cytokines as described above, in order to profile the cytokine-elaboration activity status of PeptideX2-13-expressing cells.

### Example 9

Peptidex2-13 Interaction with Human and Murine Toll-Like Receptors (Tlrs)

PeptideX2-13 (SEQ ID NO: 3) or its K2R/K12R variant (SEQ ID NO: 10) (200 μg/mL) is tested for its effects on human TLR-driven NF-κB activation in HEK293 cell lines transfected with human TLR2, 3, 4, 5, 7, 8 or 9 (InvivoGen, San Diego, CA). HEK293 cells (50,000 to 75,000 cells/well) expressing human TLR2, 3, 4, 5, 7, 8 or 9 are plated in wells of a 96-well plate (200 μl total volume) containing either PeptideX2-13 (or variant) (in 20 μl buffer), or buffer only (20 μl) as a vehicle control. All cells contain a reporter construct having coding sequence for Secreted Embryonic Alkaline Phosphatase (SEAP) under the control of a promoter inducible by the transcription of NF-κB, and are incubated in media containing a detectable SEAP expression indicator (InvivoGen, San Diego, CA). After 16-20 hours incubation at 37° C., the optical density of culture supernatants is read at 650 nm on a Molecular Devices SpectraMax™ 340PC absorbance detector.

The relative potency of PeptideX2-13 (SEQ ID NO: 3) or variant (e.g., SEQ ID NO: 10) induction of human TLR-mediated NF-κB activation is compared to the effects of known PAMP ligands that have activity as agonists for each of the human TLRs TLR2, TLR3, TLR4, TLR5, TLR7, TLR8 and TLR9, using the NF-κB-driven SEAP reporter assay (InvivoGen, San Diego, CA). The known TLR agonists are as follows: for TLR2, heat killed Listeria monocytogenes (HKLM) at 10⁸ cells/ml; for TLR3, poly (I: C) at 1 μg/ml; for TLR4, E. coli K12 LPS at 100 ng/ml; for TLR5, S. typhimurium flagellin at 100 ng/ml; for TLR7, CL097 at 1 μg/ml; for TLR8, CL075 at 1 μg/ml; for TLR9, CpG ODN 2006 at 100 ng/ml.

PeptideX2-13 (200 μg/mL) is also tested for its effects on murine TLR-driven NF-κB activation in HEK293 cell lines transfected with murine TLR2, 3, 4, 5, 7, 8 or 9 (InvivoGen, San Diego, CA) essentially as described above.

### Example 10

### In Vivo Sepsis Models

A. Cecal Ligation and Puncture Model. The observation that pentide YLQMN (SEQ ID NO: 5)-containing immunomodulatory polypeptides such as PeptideX2-13 (SEQ ID NO: 3) deliver a biological signal via specific recognition of murine TLR2 and/or TLR4 is exploited in the cecal ligation and puncture (CLP) method, a recognized model for sepsis (Kasten et al., 2010 Infect. Immun. 78:4714).

C57BL/6 (WT) mice between 6 and 8 weeks of age (20-28 gms) are obtained from Jackson Laboratory, Bar Harbor, ME; all experiments involving animals are performed under protocols approved by the Institutional Animal Care and Use Committee (IACUC). Polymicrobial sepsis is induced using the CLP method. Briefly, well fed mice are anesthetized to effect by 2.5% isoflurane in oxygen via face masks. After laparotomy, the latter 80% of the cecum is ligated and punctured once on the anti-mesenteric side with a 23-gauge needle. A small amount of bowel content is extruded through the puncture hole to ensure full thickness of the perforation. The cecum is replaced to its original location, and the midline incision is closed by a two-layer suture.

Prior to closure of the peritoneum with one figure-of-eight stitch, 0, 25, 125, 250, or 500 ug quantities of PeptideX2-13 [SEQ ID NO: 3] (each in a total volume 250 μl) are injected into the peritoneum or an equivalent volume of saline so that the total concentration in such test animals is 0, 10, 50, 100, or 200 μg/ml respectively. Sham-operated animals receive midline laparotomies, exteriorization of the cecum with prompt replacement, and closure of incisions in two layers. The animals are resuscitated with 1 ml of sterile saline subcutaneously and kept on a heating blanket and additional oxygen supply for 1 hr. Mice receiving 0, 10, 50, 100, or 200 μg/ml of PeptideX2-13 (total volume 250 μl) continue to receive this same dosing as subcutaneous injections daily starting 24 hours after the laparotomy and for which the total volume is the same for all test mice. Sham-treated mice receive daily saline subcutaneous injections of the same volume as used in the test mice starting 24 hours after the laparotomy. In survival studies, animals are given ad libitum access to food and water and followed until death or humane sacrifice per protocol. Animals are evaluated every 12 hr following CLP.

In separate test animals PeptideX2-13 is also administered at the various dosing amounts as described above but at delayed time periods post cecal ligation and puncture, using subcutaneous dosing every 24 hours to determine if the product can be lifesaving in later stages of sepsis. Pharmacokinetics of PeptideX2-13 in vivo, quantification of total circulating white blood cells and of neutrophils and CD4+ and CD8+ T-cell subsets, and quantification of cytokines, are undertaken according to standard methodologies.

B. Pneumococcal Pneumonia Model. Sepsis is often associated with severe pneumonia, and pneumococcal PAMPs for TLR2 (lipoteichoic acid, lipopeptides) and TLR4 (pneumolysis) have been identified. Accordingly, an established murine pneumococcal pneumonia model is also used to observe immunomodulatory effects of the herein described PeptideX2-13 and related immunomodulatory polypeptides, including effects of these immunomodulatory polypeptides that derive from their ability to compete with PAMPs as ligands for TLR2 and TLR4 and to deliver attenuated TLR2/4 biological signaling. Briefly, Streptococcus pneumoniae loads (serotype 3) are administered intratracheally to experimental animals before, during or after administration of PeptideX2-13 (control animals receive no PeptideX2-13; test groups receiving antibiotics alone or in addition to PeptideX2-13 are also contemplated). One or more biological indicia of infection, immunity, inflammation, and/or sepsis are determined at various timepoints, including animal survival, bacterial levels in the blood and lungs, lung histology, inflammatory mediators, appearance of anti-bacterial antibodies, SIRS-like factors, and other criteria (e.g., Chiavolini et al., 2008 Clin. Microbiol. Rev. 21 (4): 666-685; see also, e.g., Christaki et al., 2010 J. Infect. Dis. 201 (8): 1250; Christaki et al., 2011 Shock 35:492).

### Example 11

Interaction of Various Peptides with Human Tlr2

This Example describes the effects of PeptideX2 KSIAYLQMNSLK (SEQ ID NO: 1), Pentide YLQMN (SEQ ID NO: 5) (also referred to as “modified peptideX2” in this example), a reversed version of modified-peptideX2 referred to as reverse-peptideX2 (NMQLY, SEQ ID NO: 16), a shortened version of modified-peptideX2 using 4 amino acids (LQMN, SEQ ID NO: 18), and a further shortened version of modified-peptideX2 with 3 amino acids (QMN, SEQ ID NO: 19) on NF-κB activation in HEK293 cell lines transfected with human TLR2, 3, 4, 5, 7, 8 or 9.

The peptides used in this Example were synthesized as a lyophilized white product (>85% purity) and stored at −20° C. until ready for use. When needed, a vial of a peptide would be solubilized in 0.2% acetic acid, DPBS, pH 6 and stored at 4° C.

Human Toll-like receptor ligand screening was performed using test samples containing a peptide of interest at 200 μg/ml. The general procedure consisted of testing for NF-κB activation in HEK293 cells expressing a given TLR. The samples were tested against seven different human TLRs (TLR2, 3, 4, 5, 7, 8, 9). The media added to the wells were designed for the detection of NF-κB induced SEAP (secreted embryonic alkaline phosphatase) expression. After 16-24 hours of incubation, the optical density (OD) was read at 650 nm on a Molecular Devices SpectraMax 340PC absorbance detector.

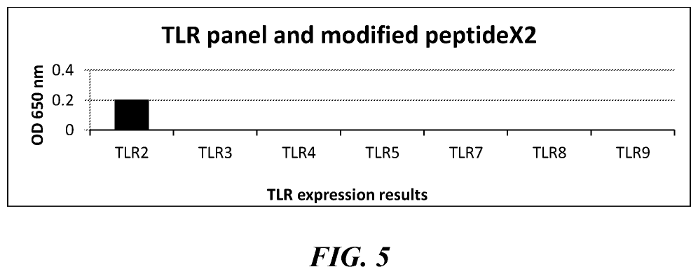

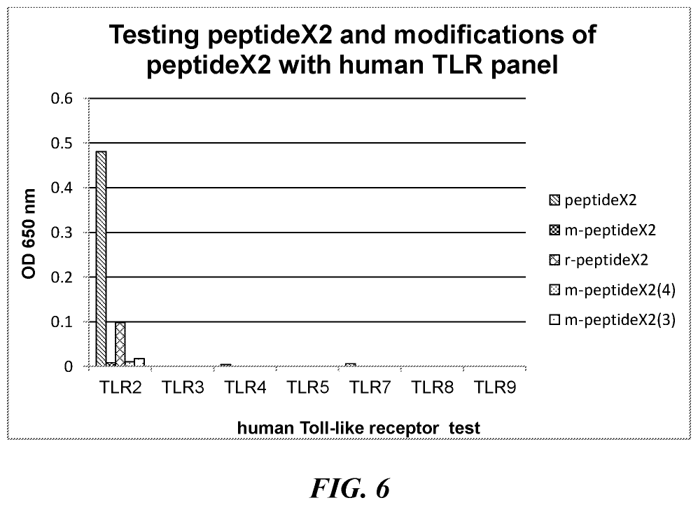

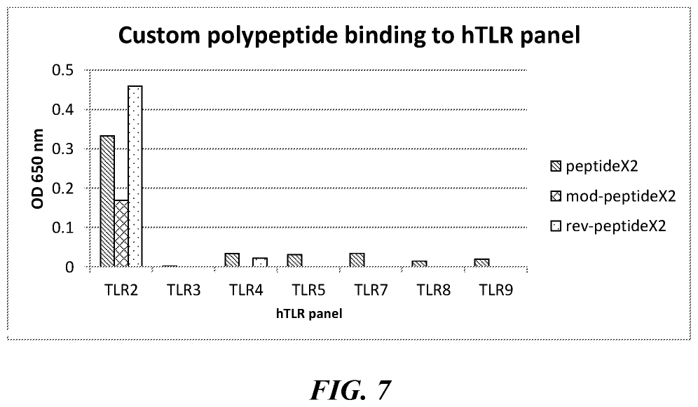

Initially, a modified version of peptideX2 was prepared consisting of YLQMN (SEQ ID NO: 5, referred to as “modified peptideX2”) and tested against human TLR panel (see FIG. 5). These results show very weak binding to TLR2 (about 3 times less activity compared to peptideX2), but when coupled with the supporting portions of this dodecapeptide, the COOH and NH2 ends provide the molecule with some improved affinity to TLR2 and some slight affinity to TLR4. These results were reproduced as shown in FIG. 6 in which various modifications of peptideX2 were constructed consisting of modified-peptideX2 (YLQMN, SEQ ID NO: 5), but also a reversed version of modified-peptideX2 known as reverse-peptideX2 (NMQLY, SEQ ID NO: 16), a shortened version of modified-peptideX2 using 4 amino acids (LQMN, SEQ ID NO: 18), and a further shortened version of modified-peptideX2 with 3 amino acids (QMN, SEQ ID NO: 19). These modified peptides were tested against the hTLR panel, and the results show weak binding only to TLR2, but only with peptideX2 and reverse-peptideX2 (where the binding of peptideX2 was greater than reverse-peptideX2) and negligible activity with other peptides. This was surprising noting that previous experiments had shown weak activity with modified peptideX2. So, another experiment was conducted comparing peptideX2 with mod-peptideX2 and rev-peptideX2 (see FIG. 7). This work supported the earlier work shown in FIG. 5, noting that modified peptideX2 did have weak binding to TLR2, and that reverse-peptideX2 had stronger binding to TLR2 than peptideX2. However, the binding in these experiments was nevertheless relatively similar, suggesting that peptideX2 and rev-peptideX2 probably operate similarly.

### Example 12

### Efficacy of Pentide in Animal Model of Rheumatoid Arthritis

This example describes the efficacy of pentide (YLQMN, SEQ ID NO: 5) in the collagen-induced Lewis rat model of rheumatoid arthritis.

6 Lewis rats (ENVIGO, female, 190-210g) were received, individually examined, and housed. No clinical signs of disease or distress were observed. The rats were placed in quarantine with daily inspections, and the rats were individually examined and found to be free of any clinical signs of disease or distress. No deaths were recorded during the quarantine period. The rats were released for routine maintenance. The rats were ear notched for identification purposes. 10 mg bovine Type II collagen (Chondrex, Cat. 20021) was dissolved in 2.5 ml 0.1M acetic acid by stirring overnight at 4-8° C. in the dark. 1.2 ml collagen was emulsified in 1.2 ml Incomplete Freund's Adjuvant (ICFA, Sigma, Cat. F5506) on an ice bath. A droplet of the emulsion floated on water did not disperse. The rats were weighed, anesthetized, shaved at the base of tail, and injected intra-dermally with 0.2 ml collagen/ICFA emulsion in two sites. The rats were returned to routine maintenance. Rat weights were recorded daily. The rats were scored for signs of arthritis daily as follows:
1) each paw receives a score
2) 0=no visible effects of arthritis
3) 1=edema and/or erythema of one digit
4)₂=edema and/or erythema of 2 joints
5) 3=edema and/or erythema of more than 2 joints
6) 4=severe arthritis of the entire paw and digits
7) Calculate Arthritic Index=add all individual paw scores
8) Maximum Arthritic Index=16

The rats were weighed, scored for signs of arthritis and assigned to various treatment groups of one or more rats to each group. For the prednisolone control group, 200.85 mg prednisolone 21-hemisuccinate sodium salt (Sigma, Cat. P4153) was dissolved in 15 ml de-ionized water to prepare a 10 mg/ml solution which was stored at 4-8° C. when not in use. Rats in the study groups were dosed daily from day 15 through day 30 according to the formula for which a rat weighing (g)×64 ml/1000 g (represents the total blood volume) multiplied by the test product dose/test product concentration to determine the total volume to be injected into the animal per day to achieve that desired estimated blood concentration. Rats were weighed and scored daily during the treatment phase of the experiment and the results recorded and reported.

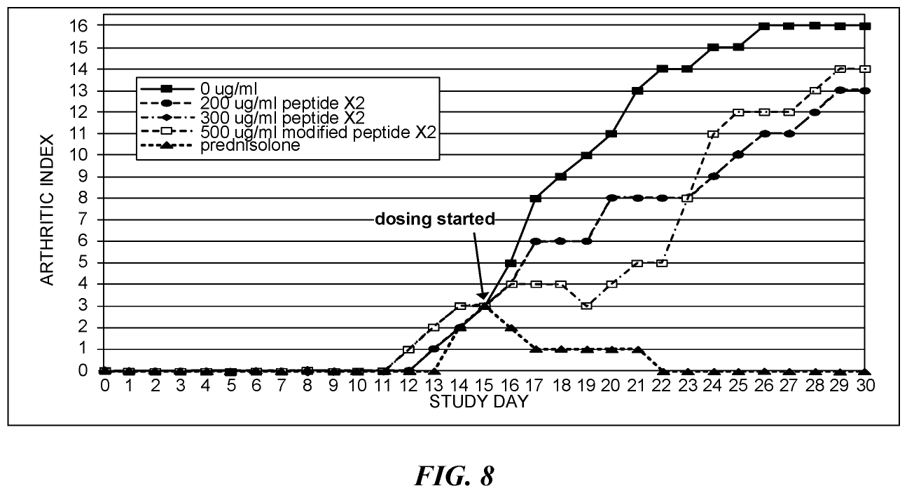

Previous results (see FIG. 1, Example 2) suggested that peptideX2 could indeed have anti-inflammatory properties at 200 μg/ml concentration. Based on that study, another study was carried out, looking at 200 and 300 μg/ml of peptideX2. A modified version of peptideX2 using the “core” of the molecule (i.e., pentide YLQMN, SEQ ID NO:5) was also included in this study. The results show that peptide X₂ (at 200 μg/ml) and the core molecule (YLQMN) (at 500 μg/ml) provided some anti-inflammatory benefit (see FIG. 8).

### Example 13

### Solubility of Various Peptides

Various modifications of peptideX2-13 were tested for solubility (see Table 2). The results show that changing the alanine (A) with other nonpolar amino acids (leucine or valine) rendered the molecule insoluble in aqueous buffer. Omitting the lysine (K) rendered the molecule (modification (7), SEQ ID NO: 23) insoluble in aqueous buffer and produces an insoluble, gel like product in an aqueous solvent. A modification of peptideX2-13 (modification (10), SEQ ID NO: 24) showed less solubility compared to peptideX2-13, and there was no significant difference when reversing the core amino acid sequence (modification (11), SEQ ID NO: 25).

TABLE 2
Modifications of peptideX2-13 and Solubility

| | | | | | | | | | | | | | | Solubility |
|---|---|---|---|---|---|---|---|---|---|---|---|---|---|---|
| Molecule: | | | | | | | | | | | | | | |
| peptideX2-13 (1) | G | K | A | A | Y | L | Q | M | N | A | A | K | G | Soluble in buffer |
| [SEQ ID NO: 3] | | | | | | | | | | | | | | (0.2% acetic acid, |
| | | | | | | | | | | | | | | 0.375M Hepes, |
| | | | | | | | | | | | | | | 0.375% glycerol, |
| | | | | | | | | | | | | | | pH 7) |
| Modifications: | | | | | | | | | | | | | | |
| (2) | G | R | A | A | Y | L | Q | M | N | A | A | R | G | Soluble in buffer |
| [SEQ ID NO: 10] | | | | | | | | | | | | | | at 2 mg/ml |
| (3) | G | K | V | V | Y | L | Q | M | N | V | V | K | G | Insoluble in |
| [SEQ ID NO: 20] | | | | | | | | | | | | | | buffer; turbid |
| | | | | | | | | | | | | | | solution |
| (4) | G | K | L | L | Y | L | Q | M | N | L | L | K | G | Insoluble in |
| [SEQ ID NO: 21] | | | | | | | | | | | | | | buffer; turbid |
| | | | | | | | | | | | | | | solution |
| (5) | P | K | A | A | Y | L | Q | M | N | A | A | K | P | Soluble in buffer |
| [SEQ ID NO: 12] | | | | | | | | | | | | | | at 2 mg/ml |
| (6) | G | K | — | — | Y | L | Q | M | N | — | — | K | G | Soluble in buffer |
| [SEQ ID NO: 22] | | | | | | | | | | | | | | at 2 mg/ml |
| (7) | G | — | — | — | Y | L | Q | M | N | — | — | — | G | Insoluble in |
| [SEQ ID NO: 23] | | | | | | | | | | | | | | buffer; gel like |
| (8) | G | K | A | A | N | M | Q | L | Y | A | A | K | G | Soluble in buffer |
| [SEQ ID NO: 17] | | | | | | | | | | | | | | at 2 mg/ml |
| (9) | G | K | A | — | Y | L | Q | M | N | — | A | K | G | Soluble in buffer |
| [SEQ ID NO: 13] | | | | | | | | | | | | | | at 2 mg/ml |
| (10) | G | R | SS | A | Y | L | Q | M | N | A | SS | R | G | Insoluble at |
| [SEQ ID NO: 24] | | | | | | | | | | | | | | 5 mg/ml |
| | | | | | | | | | | | | | | concentration |
| (11) | G | R | SS | A | N | M | Q | L | Y | A | SS | R | G | Insoluble at |
| [SEQ ID NO: 25] | | | | | | | | | | | | | | 5 mg/ml |
| | | | | | | | | | | | | | | concentration |
| (12) | G | K | A | A | Y | L | Q | M | N | A | A | K | P | |
| [SEQ ID NO: 14] | | | | | | | | | | | | | | |
| (13) | P | K | A | A | Y | L | Q | M | N | A | A | K | G | |
| [SEQ ID NO: 15] | | | | | | | | | | | | | | |

The various embodiments described above can be combined to provide further embodiments. All of the U.S. patents, U.S. patent application publications, U.S. patent application, foreign patents, foreign patent application and non-patent publications referred to in this specification and/or listed in the Application Data Sheet, including U.S. Provisional Patent Application No. 63/356,867, filed on Jun. 29, 2022, are incorporated herein by reference, in their entirety. Aspects of the embodiments can be modified, if necessary to employ concepts of the various patents, application and publications to provide yet further embodiments. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

### ADDITIONAL REFERENCES

Ameziane N, Beillat T, Verpillat P, et al. Association of the Toll-like receptor 4 gene Asp299gly polymorphism with acute coronary events. Arterioscler Thromb Vasc Biol 23 (12): e61-e64, 2003
Andersson U, Wang H, Palmblad K, et al. High mobility group 1 protein (HMG-1) stimulates proinflammatory cytokine synthesis in human monocytes. J Exp Med 192 (4), 565-570, 2000
Bieber J D, Terkeltaub R A. Gout: on the brink of novel therapeutic options for an ancient disease. Arthritis Rheum 50 (8): 2400-2414, 2004
Brentano F, Schorr O, Gay R E, et al. RNA released from necrotic synovial fluid cells activates rheumatoid arthritis synovial fibroblasts via Toll-like receptor 3. Arthritis Rheum 52 (9): 2656-2665, 2005
Bjorkbacka H, Junjathoor V V, Moore KJ, et al. Reduced atherosclerosis in MyD88-null mice links elevated serum cholesterol levels to activation of innate immunity signaling pathways. Nat Med 10 (4): 416-421, 2004
Chen T, Rimpilainen M, Luukkainen R, et al. Bacterial components in the synovial tissue of patients with advanced rheumatoid arthritis or osteoarthritis: analysis with gas chromatography-mass spectrometry and pan-bacterial polymerase chain reaction. Arthritis Rheum 49 (3): 328-334, 2003
Choe J Y, Crain B, Wu S R, et al. Interleukin-1 receptor dependence of serum transferred arthritis can be circumvented by Toll-like receptor 4 signaling. J Exp Med 197 (4): 537-542, 2003
Cromartie W J, Craddock J G, Schwab J H, et al. Arthritis in rats after systemic injection of streptococcal cells or cell walls. J Exp Med 146 (6): 1585-1602, 1977
Drexler S K, Sacre S M, Foxwell B M. Toll-like receptors: a new target in rheumatoid arthritis. Expert Rev. Clin. Immunol. 2 (4), 585-599 (2006)
Elhage R, Jawien J, Rudling M, et al. Reduced atherosclerosis in interleukin-18 deficient apolipoprotein E-knockout mice. Cardiovasc Res 59 (1): 234-240, 2003
Fiuza C, bustin M, Talwar S, et al. Inflammation-promoting activity of HMGB1 on human microvascular endothelial cells. Blood 101 (7): 2652-2660, 2003
Gerard H C, Wang Z, Wang G F, et al. Chromosomal DNA from a variety of bacterial species is present in synovial tissue from patients with various forms of arthritis. Arthritis Rheum 44 (7): 1689-1697, 2001
Gewirtz A T, Vijay-Kumar M, Brant S R, et al. Dominant-negative TLR5 polymorphism reduces adaptive immune response to flagellin and negatively associates with Crohn's disease. Am J Physiol Gastrointest Liver Physiol 290 (6): G1157-G1163, 2006
Girardin S E, Boneca I G, Viala J, et al. Nod2 is a general sensor of peptidoglycan through muramyl dipeptide (MDP) detection. J Biol Chem 278 (11): 8869-8872, 2003
Gritz D C, Wong I G. Incidence and Prevalence of Uveitis in Northern California; the Northern California Epidemiology of Uveitis Study. Ophthalmology 111 (3): 491-500, 2004
Harnett W, Harnett M M, Bryon O. Structural/functional aspects of ES-62-a secreted immunomodulatory phosphorylcholine-containing filarial nematode glycoprotein. Curr Protein Pept Sci 4 (1): 59-71, 2003
Hugot J P, Chamaillard M, Zouali H, et al. Association of NOD2 leucine rich repeat variants with susceptibility to Crohn's disease. Nature 411 (6837): 599-603, 2001
Inohara N, Ogura Y, Fontalba A, et al. Host recognition of bacterial muramyl dipeptide mediated through NOD2. Implications for Crohn's disease. J Biol Chem 278 (8): 5509-5512, 2003
Iwahashi M, Yamamura M, Aita T et al. Expression of Toll-like receptor 2 on CD16+blood monocytes and synovial tissue macrophages in rheumatoid arthritis. Arthritis Rheum 50 (5): 1457-1467 (2004)
Jabs D A, Rosenbaum J T, Foster C S, et al. Guidelines for the Use of Immunosuppressive Drugs in Patients with Ocular Inflammatory Disorders: Recommendations of an Expert Panel. Am J Ophthal 130 (4): 492-513, 2000
Jiang D, Liang J, Fan J, et al. Regulation of lung injury and repair by Toll like receptors and hyaluronan. Nat Med 11 (11): 1173-1179, 2005
Joosten L A, Koenders M I, Smeets R L, et al. Toll-like receptor 2 pathway drives streptococcal cell wall-induced joint inflammation: critical role of myeloid differentiation factor 88. J Immunol 171 (11): 6145-6153, 2003
Kawasaki T, Kawai T. Toll-like receptor signaling pathways. Frontiers in Immunology. Vol 5:1-8, September 2014
Kempsell K E, Cox C J, Hurle M, et al. Reverse transcriptase-PCR analysis of bacterial rRNA for detection and characterization of bacterial species in arthritis synovial tissue. Infect Immunol 68 (1): 6012-6026, 2000
Kiechl S, Lorenz E, Reindl M, et al. Toll like receptor 4 polymorphisms and atherogenesis. NEJM 347 (3): 185-192, 2002
Kilding R, Akil M, Till S, et al. A biologically important single nucleotide polymorphism within the Toll-like receptor 4 gene is not associated with rheumatoid arthritis. Clin Exp Rheumatol 21 (3): 340-342, 2002
Kirii H, Niwa T, Yamada Y, et al. Lack of interleukin-1b decreases the severity of atherosclerosis in ApoE-deficient mice. Arterioscler Thromb Vasc Biol 23 (4): 656-660, 2003
Kokkola R, Sundberg E, Ulfgren A K, et al. High mobility group box chromosomal protein 1: a novel proinflammatory mediator in synovitis. Arthritis Rheum 46 (1): 2598-2603,
Kyburz D, Rethage J, Seibl R, et al. Bacterial peptidoglycans but not CpG oligodeoxynucleotides activate synovial fibroblasts by Toll-like receptor signaling. Arthritis Rheum 48 (3): 642-650, 2003
Lacerte P, Brunet A, Egarnes B, et al. Overexpression of TLR2 and TLR9 on m onocyte subsets of active rheumatoid arthritis patients contributes to enhance responsiveness to TLR agonists. Arthritis Research & Therapy (2016) 18:10
Lamb R M, Zeggini E, Thomson W, et al. Toll-like receptor (TLR4) gene polymorphisms and susceptibility to juvenile idiopathic arthritis. Ann Rheum Dis 64 (5): 767-769, 2004
Lee G. Drug Development Using Natural Toxins. Toxins 13 (414): 1-3, 2021
Lee J Y, Sohn K H, Rhee S H, et al. Saturated fatty acids, but not unsaturated fatty acids, induce the expression of cyclooxygenase-2 mediated through Toll-like receptor 4. J Biol Chem 276 (20): 16683-16689, 2001
Lee J Y, Zhao L, Youn H S, et al. Saturated fatty acid activates but polyunsaturated fatty acid inhibits Toll like receptor 2 dimerized with Toll-like receptor 6 or 1. J Biol Chem 279 (17): 16971-16979, 2004
Libby P. Inflammation in atherosclerosis. Nature 420 (6917): 868-874, 2002
Lima C X, Souza D G, Amaral F A, et al. Therapeutic Effects of Treatment with Anti-TLR2 and Anti-TLR4 Monoclonal Antibodies in Polymicrobial Sepsis. PLOS ONE 10 (7): e0132336, 2015
Liu-Bryan R, Pritzker K, Firestein G S, et al. TLR2 signaling in chondrocytes drives calcium pyrophosphate dihydrate and monosodium urate crystal-induced nitric oxide generation. J Immunol 174 (8): 5016-5023, 2005
Liu-Bryan R, Scott P, Sydlaske A, et al. Innate immunity conferred by Toll like receptors 2 and 4 and myeloid differentiation factor 88 expression is pivotal to monosodium urate monohydrate crystal induced inflammation. Arthritis Rheum 52 (9): 2936-2946, 2005
Liu Z Q, Deng G M, Foster S, et al. Staphylococcal peptidoglycans induce arthritis. Arthritis Res 3 (6): 375-380, 2001
Mariathasan S, Newton K, Monack D M, et al. Differential activation of the inflammasome by caspase-1 adaptors ASC and Ipaf. Nature 430 (6996): 213-218, 2004
Martinon F, Agostini L, Meylan E, et al. Identification of bacterial muramyl dipeptide as activator of the NALP3/cryopyrin inflammasome. Curr Biol 14 (21): 1929-1934, 2004
Martinon F, Burns K, Tschopp J. The inflammasome: a molecular platform triggering activation of inflammatory caspases and processing of proIL-B. Mol Cell 10 (2): 417-426, 2002
Martinon F, Petrilli V, Mayor A, et al. Gout-associated uric acid crystals activate the NALP3 inflammasome. Nature 440 (7081): 237-241, 2006
McCarty, D J. Crystals and arthritis. Dis Mon 40 (6): 255-299, 1974
McGarry T, Biniecka M, Gao W, et al. Resolution of TLR2-induced inflammation through manipulation of metabolic pathways in Rheumatoid Arthritis. Nature Scientific Reports 7:43165 (2017)
McGarry T, Veale D J, Gao W, et al. Toll-like receptor 2 (TLR2) induces migration and invasive mechanisms in rheumatoid arthritis. Arthritis Research & Therapy 2015; 17:153
Meng G, Rutz M, Schiemann M, Metzger J, et al. Antagonistic antibody prevents toll-like receptor 2-driven lethal shock-like syndromes. J Clin Invest 113:1473-1481, 2004
Michelsen K S, Wong M H, Shah P K, et al. Lack of Toll like receptor 4 or myeloid differentiation factor 88 reduces atherosclerosis and laters plaque phenotype in mice deficient in apolipoprotein E. PNAS 101 (29): 10679-10684, 2004
Miller Y I, Viriyakosol S, binder C J, et al. Minimally modified LDL binds to CD14, induces macrophage spreading via TLR4/MD-2, and inhibits phagocytosis of apoptotic cells. J Biol Chem 278 (3): 1561-1568, 2003
Netea M G, Hijmans A, van Wissen S, et al. Toll-like receptor-4 Asp299Gly polymorphism does not influence progression of atherosclerosis in patients with familial hypercholesterolemia. Eur J Clin Invest 34 (2): 94-99, 2004
Nishina P M, Wang J, Toyofuku W, et al. Atherosclerosis and plasma and liver lipids in nine inbred strains of mice. Lipids 28 (7): 599-605, 1993
Ogura Y, Bonen D K, Inohara N, et al. A frameshift mutation in NOD2 associated with susceptibility to Crohn's disease. Nature 411 (6837): 603-606, 2001
O'Neill L A, Bryant C E, Doyle S L. Therapeutic Targeting of Toll-Like Receptors for Infectious and Inflammatory Diseases and Cancer. Pharmacol Rev 61:177-197, 2009
Ospelt C, Kyburz D, Pierer M, et al. Toll-like receptors in rheumatoid arthritis joint destruction mediated by two distinct pathways. Ann Rheum Dis 2004; 63:90-91
Park J S, Arcaroli J, Yum H K et al. Activation of gene expression in human neutrophils by high mobility group box 1 protein. Am J Physiol cell Physiol 284 (4): C870-C879, 2003
Park J S, Gamboni-Robertson F, He Q, et al. High mobility group box 1 protein interacts with multiple Toll-like receptors. Am J Physiol Cell Physiol 290 (3): C917-C924, 2006
Pierer M, Rethage J, Seibl R, et al. Chemokine secretion of rheumatoid arthritis synovial fibroblasts stimulated by Toll-like receptor 2 ligands. J Immunol 172 (2): 1256-1265, 2004
Radstake T R, Roelofs M F, Jenniskens Y M. Expression of Toll-like receptors 2 and 4 in rheumatoid synovial tissue and regulation of proinflammatory cytokines interleukin-12 and interleukin-18 via interferon-g. Arthritis Rheum 50 (12), 3856-3865, 2004
Rakoff-Nahoum S, Paglino J, Eslami-Varzaneh F, et al. Recognition of commensal microflora by Toll like receptor sis required for intestinal homeostasis. Cell 118 (2): 229-241, 2004
Reddick R L, Zhang S H, Maeda N. Atherosclerosis in mice lacking apo E. Evaluation of lesional development and progression. Arterioscler. Thromb 14 (1): 141-147, 1994
Roelofs M F, Boelens W C, Joosten L A, et al. Identification of small heat shock protein B8 (HSP22) as a novel TLR4 ligand and potential involvement in the pathogenesis of rheumatoid arthritis. J Immunol 176 (11): 7021-7027, 2006
Roelofs M F, Joosten L A, Abdollahi-Roodsaz S, et al. The expression of Toll-like receptors 3 and 7 in rheumatoid arthritis synovium is increased and costimulation of Toll-like receptors 3, 4, and 7/8 results in synergistic cytokine production by dendritic cells. Arthritis Rheum 52 (8): 2313-2322, 2005
Sanchez E, Orozco G, Lopez-Nevot M A, et al. Polymorphisms of Toll-like receptor 2 and 4 genes in rheumatoid arthritis and systemic lupus erythematosus. Tissue Antigens 63 (1): 54-57, 2004
Scherr G, Redlich K, Wu Q, et al. Enhanced expression of heat shock protein 70 (HSP70) and heat shock factor 1 (HSF1) activation in rheumatoid arthritis synovial tissue. Differential regulation HSP70 expression and HSF1 activation in synovial fibroblasts by proinflammatory cytokines, shear stress, and anti-inflammatory drugs. J Clin Invest 102 (2): 302-311, 1998
Schumacher H R Jr, Arayssi T, Crane M, et al. Chlamydia trachomatis nucleic acids can be found in the synovium of some asymptomatic subjects. Arthritis Rheum 42 (6): 1281-1284 (1999)
Scott D L, Delamere J P, Walton K W. The distribution of fibronectin in the pannus in rheumatoid arthritis. Br J Exp Pathol 62 (4): 362-368, 1981
Scoville C D, Rasmussen D. Description and characterization of a unique human immunoglobulin G1 kappa idiotype found in placental tissue. Placenta 50:1-10 (2017)
Seibl R, Birchler T, Loeliger S, et al. Expression and Regulation of Toll-like Receptor 2 in Rheumatoid Arthritis Synovium. Am J Pathology 162:1221 (2003)
Sen H N, Vitale S, Gangaputra S S, et al. Periocular Corticosteroid Injections in Uveitis: Effects and Complications. Ophthal 121 (11): 2275-2286, 2014
Taniguchi N, Kawahara K, Yone K, et al. High mobility group box chromosomal protein 1 plays a role in the pathogenesis of rheumatoid arthritis as a novel cytokine. Arthritis Rheum 48 (4): 971-981, 2003
Ting J P, Kastner D L, Hoffman H M. CATERPILLERs, pyrin and hereditary immunological disorders. Nat Rev Immunol 63 (3): 183-195, 2006
Torok H P, Glas J, Tonenchi L, et al. Crohn's disease is associated with Toll-like receptor-9 polymorphism. Gastroenterology 127 (1): 365-366, 2004
Ultaigh SNA, Saber T P, McCormick J, et al. Blockade of Toll-like receptor 2 prevents spontaneous cytokine release from rheumatoid arthritis ex vivo synovial explant cultures. Arthritis Research & Therapy 2011, 13: R33
Van der Heijden I M, Wilbrink B, Tcherverikov I, et al. Presence of bacterial DNA and bacterial peptidoglycans in joints of patients with rheumatoid arthritis and other arthritides. Arthritis Rheum 43 (3): 593-598, 2000
Van Heel D A, Ghosh S, butler M, et al. Synergistic enhancement of Toll-like receptor response by NOD1 activation. Eur J Immunol 35 (8): 2471-2476, 2005
Walton K A, Hsieh X, Gharavi N, et al. Receptors involved in the oxidized 1-palmitoy-2-arachidonoyl-sn-glycero-3-phosphorylcholine-mediated synthesis of interleukin-8. A role for Toll-like receptor 4 and a glycosylphosphatidylinositol-anchored protein. J Biol Chem 278 (32): 29661-29666, 2003
Wang H, Bloom O, Zhang M, et al. HMG-1 as a late mediator of endotoxin lethality in mice. Science 285 (5425): 248-251, 1999
Watanabe T, Kitani A, Murray P J, et al. NOD2 is a negative regulator of Toll-like receptor 2 mediated T helper type 1 responses. Nat Immunol 5 (8): 800-808, 2004
Wright S D, Burton C, Hernandez M, et al. Infectious agents are not necessary for murine atherogenesis. J Exp Med 191 (8): 1437-1442, 2000
Yu D, Rumore P M, Liu Q, et al. Soluble oligonucleosomal complexes in synovial fluid from inflamed joints. Arthritis Rheum 40 (4): 648-654, 1997

What is claimed is:

1. An isolated immunomodulatory polypeptide that is
(a) no more than 31, 30, 29, 28, 27, 26, 25, 24, 23, 22, 21, 20, 19, 18, 17, 16, 15, 14, 13, 12, 11, 10, or 9 amino acids in length, comprising or consisting of the amino acid sequence X₁X₂X₃X₄YLQMNX₅X₆X₇X₈ as set forth in SEQ ID NO: 11, wherein X₁ and X₈ are each independently glycine (G) or proline (P), X₂ and X₇ are each independently lysine (K) or arginine (R), and X₃, X₄, X₅ and X₆ are each independently alanine (A) or nothing,
(b) YLQMN as set forth in SEQ ID NO:5,
(c) NMQLY as set forth in SEQ ID NO:16, or
(d) no more than 31, 30, 29, 28, 27, 26, 25, 24, 23, 22, 21, 20, 19, 18, 17, 16, 15, 14, or 13 amino acids in length, comprising or consisting of the amino acid sequence GKAANMQLYAAKG as set forth in SEQ ID NO:17.

2. The isolated immunomodulatory polypeptide of claim 1, comprising or consisting of the amino acid sequence selected from the group consisting of:
(a) GKAAYLQMNAAKG as set forth in SEQ ID NO:3,
(b) GRAAYLQMNAARG as set forth in SEQ ID NO:10,
(c) PKAAYLQMNAAKP as set forth in SEQ ID NO:12,
(d) GKAYLQMNAKG as set forth in SEQ ID NO:13,
(e) GKAAYLQMNAAKP as set forth in SEQ ID NO:14,
(f) PKAAYLQMNAAKG as set forth in SEQ ID NO:15, and
(g) GKYLQMNKG as set forth in SEQ ID NO: 22.

3. A pharmaceutical composition, comprising the immunomodulatory polypeptide of claim 1 or claim 2 and a physiologically acceptable carrier.

4. A fusion protein comprising the immunomodulatory polypeptide of claim 1 or claim 2 fused to a fusion polypeptide domain.

5. A pharmaceutical composition comprising the fusion protein of claim 4; and a physiologically acceptable carrier.

6. An immunomodulatory polypeptide for use as a medicament, wherein the immunomodulatory polypeptide is
(a) no more than 31, 30, 29, 28, 27, 26, 25, 24, 23, 22, 21, 20, 19, 18, 17, 16, 15, 14, 13, 12, 11, 10, or 9 amino acids in length, comprising or consisting of the amino acid sequence X₁X₂X₃X₄YLQMNX₅X₆X₇X₈ as set forth in SEQ ID NO: 11, wherein X₁ and X₈ are each independently glycine (G) or proline (P), X₂ and X₇ are each independently lysine (K) or arginine (R), and X₃, X₄, X₅ and X₆ are each independently alanine (A) or nothing,
(b) YLQMN as set forth in SEQ ID NO:5,
(c) NMQLY as set forth in SEQ ID NO:16, or
(d) no more than 31, 30, 29, 28, 27, 26, 25, 24, 23, 22, 21, 20, 19, 18, 17, 16, 15, 14, or 13 amino acids in length, comprising or consisting of the amino acid sequence GKAANMQLYAAKG as set forth in SEQ ID NO:17.

7. An immunomodulatory polypeptide for use in the treatment of TLR2-mediated sepsis and/or TLR4-mediated sepsis, wherein the immunomodulatory polypeptide is
(a) no more than 31, 30, 29, 28, 27, 26, 25, 24, 23, 22, 21, 20, 19, 18, 17, 16, 15, 14, 13, 12, 11, 10, or 9 amino acids in length, comprising or consisting of the amino acid sequence X₁X₂X₃X₄YLQMNX₅X₆X₇X₈ as set forth in SEQ ID NO: 11, wherein X₁ and X₈ are each independently glycine (G) or proline (P), X₂ and X₇ are each independently lysine (K) or arginine (R), and X₃, X₄, X₅ and X₆ are each independently alanine (A) or nothing,
(b) YLQMN as set forth in SEQ ID NO:5,
(c) NMQLY as set forth in SEQ ID NO:16, or
(d) no more than 31, 30, 29, 28, 27, 26, 25, 24, 23, 22, 21, 20, 19, 18, 17, 16, 15, 14, or 13 amino acids in length, comprising or consisting of the amino acid sequence GKAANMQLYAAKG as set forth in SEQ ID NO:17.

8. An immunomodulatory polypeptide for use in the treatment of one or more of conditions (a)-(l), wherein the immunomodulatory polypeptide is
(a) no more than 31, 30, 29, 28, 27, 26, 25, 24, 23, 22, 21, 20, 19, 18, 17, 16, 15, 14, 13, 12, 11, 10, or 9 amino acids in length, comprising or consisting of the amino acid sequence X₁X₂X₃X₄YLQMNX₅X₆X₇X₈ as set forth in SEQ ID NO: 11, wherein X₁ and X₈ are each independently glycine (G) or proline (P), X₂ and X₇ are each independently lysine (K) or arginine (R), and X₃, X₄, X₅ and X₆ are each independently alanine (A) or nothing,
(b) YLQMN as set forth in SEQ ID NO:5,
(c) NMQLY as set forth in SEQ ID NO:16, or
(d) no more than 31, 30, 29, 28, 27, 26, 25, 24, 23, 22, 21, 20, 19, 18, 17, 16, 15, 14, or 13 amino acids in length, comprising or consisting of the amino acid sequence GKAANMQLYAAKG as set forth in SEQ ID NO:17, and
wherein the condition is selected from:
(a) graft rejection to be decreased in a graft transplant recipient,
(b) the graft rejection of (a) wherein the graft transplant is selected from kidney, heart, liver, pancreas and lung,
(c) graft versus host disease in a bone marrow transplant recipient,
(d) preeclampsia or hemolysis-elevated liver enzymes-low platelet count (HELLP) syndrome,
(e) severe preeclampsia,
(f) rheumatoid arthritis,
(g) a malignant condition,
(h) the malignant condition of (g) which is selected from breast cancer, ovarian cancer, adenoma, colorectal carcinoma, gastric carcinoma, lung carcinoma, prostate carcinoma, hepatocellular carcinoma, melanoma, leukemia and lymphoma,
(i) an autoimmune disease,
(j) the autoimmune disease of (i) which is selected from rheumatoid arthritis, psoriatic arthritis, ulcerative colitis, Crohn's disease, seronegative spondyloarthopathies, systemic lupus erythematosus, Behcet's disease and vasculitis,
(k) atherosclerosis,
(l) uveitis.

9. A method of inducing a peripheral blood white cell response that includes cellular release of at least one of IL-6, IL-10 and TNFα, comprising contacting one or a plurality of peripheral blood white cells in vitro or in vivo with the immunomodulatory polypeptide of either claim 1 or claim 2, under conditions and for a time sufficient to induce detectable cellular release of at least one of IL-6, IL-10 and TNFα.

10. A method of treating an organ to be transplanted into an allogeneic recipient to reduce a likelihood or severity of allograft rejection by the recipient, comprising contacting the organ with the immunomodulatory polypeptide of either claim 1 or claim 2, under conditions and for a time sufficient to reduce the likelihood or severity of allograft rejection.

11. A method of selectively labeling a mammalian peripheral blood white cell neutrophil subpopulation, comprising contacting a population of mammalian peripheral blood white cells which comprises neutrophils with the immunomodulatory polypeptide of either claim 1 or claim 2, wherein either (i) the immunomodulatory polypeptide comprises a detectable label or (ii) the immunomodulatory polypeptide is indirectly detected.

12. The method of claim 11, wherein the detectable label is selected from the group consisting of a fluorescent dye, a radioactive substance and a metal particle.

13. A method for treating, reducing severity of, or reducing likelihood of occurrence of TLR2-mediated sepsis and/or TLR4-mediated sepsis in a subject, comprising administering to the subject a therapeutically effective amount of an immunomodulatory polypeptide that is
(a) no more than 31, 30, 29, 28, 27, 26, 25, 24, 23, 22, 21, 20, 19, 18, 17, 16, 15, 14, 13, 12, 11, 10, or 9 amino acids in length, comprising or consisting of the amino acid sequence X₁X₂X₃X₄YLQMNX₅X₆X₇X₈ as set forth in SEQ ID NO: 11, wherein X₁ and X₈ are each independently glycine (G) or proline (P), X₂ and X₇ are each independently lysine (K) or arginine (R), and X₃, X₄, X₅ and X₆ are each independently alanine (A) or nothing,
(b) YLQMN as set forth in SEQ ID NO:5,
(c) NMQLY as set forth in SEQ ID NO:16, or
(d) no more than 31, 30, 29, 28, 27, 26, 25, 24, 23, 22, 21, 20, 19, 18, 17, 16, 15, 14, or 13 amino acids in length, comprising or consisting of the amino acid sequence GKAANMQLYAAKG as set forth in SEQ ID NO:17.

14. A method of treating a patient, comprising administering to the patient a therapeutically effective amount of an immunomodulatory polypeptide that is
(a) no more than 31, 30, 29, 28, 27, 26, 25, 24, 23, 22, 21, 20, 19, 18, 17, 16, 15, 14, 13, 12, 11, 10, or 9 amino acids in length, comprising or consisting of the amino acid sequence X₁X₂X₃X₄YLQMNX₅X₆X₇X₈ as set forth in SEQ ID NO: 11, wherein X₁ and X₈ are each independently glycine (G) or proline (P), X₂ and X₇ are each independently lysine (K) or arginine (R), and X₃, X₄, X₅ and X₆ are each independently alanine (A) or nothing,
(b) YLQMN as set forth in SEQ ID NO:5,
(c) NMQLY as set forth in SEQ ID NO:16, or
(d) no more than 31, 30, 29, 28, 27, 26, 25, 24, 23, 22, 21, 20, 19, 18, 17, 16, 15, 14, or 13 amino acids in length, comprising or consisting of the amino acid sequence GKAANMQLYAAKG as set forth in SEQ ID NO:17,
wherein the method is selected from:
(a) a method for treating, reducing severity of, or reducing likelihood of occurrence of TLR2-mediated sepsis in the patient,
(b) a method of decreasing graft rejection wherein the patient is a transplant patient,
(c) the method of (b) wherein the transplant is selected from kidney, heart, liver, pancreas and lung,
(d) a method of treating or decreasing graft versus host disease wherein the patient is a bone marrow transplant patient,
(e) a method of treating preeclampsia or hemolysis-elevated liver enzymes-low platelet count (HELLP) syndrome in the patient,
(f) the method of (e) wherein the patient has been diagnosed with severe preeclampsia,
(g) a method of treating rheumatoid arthritis in the patient,
(h) a method of treating a malignant condition in the patient,
(i) the method of (h) wherein treating the malignant condition comprises at least one of killing a tumor cell and inhibiting metastasis,
(j) the method of (h) wherein the malignant condition is selected from breast cancer, ovarian cancer, adenoma, colorectal carcinoma, gastric carcinoma, lung carcinoma, prostate carcinoma, hepatocellular carcinoma, melanoma, leukemia and lymphoma,
(k) a method of treating an autoimmune disease in the patient,
(l) the method of (k) wherein the autoimmune disease is selected from rheumatoid arthritis, psoriatic arthritis, ulcerative colitis, Crohn's disease, seronegative spondyloarthopathies, systemic lupus erythematosus, Behcet's disease and vasculitis,
(m) a method of treating atherosclerosis, and
(n) uveitis.

15. The isolated immunomodulatory polypeptide of claim 1 or 2, the pharmaceutical composition of claim 3 or claim 5, the fusion protein of claim 4, the immunomodulatory polypeptide of any one of claims 6-8, or the method of any one of claims 9-14, where the immunomodulatory polypeptide comprises or consists of the amino acid sequence GKAAYLQMNAAKG as set forth in SEQ ID NO: 3, consists of YLQMN as set forth in SEQ ID NO: 5, or consists of NMQLY as set forth in SEQ ID NO:16.

16. An isolated polynucleotide comprising a nucleic acid sequence that encodes the immunomodulatory polypeptide of claim 1 or claim 2.

17. An expression vector comprising the polynucleotide of claim 16.

18. A host cell transformed or transfected with the expression vector of claim 17.

19. A method of producing an immunomodulatory polypeptide that is
(a) no more than 31, 30, 29, 28, 27, 26, 25, 24, 23, 22, 21, 20, 19, 18, 17, 16, 15, 14, 13, 12, 11, 10, or 9 amino acids in length, comprising or consisting of the amino acid sequence X₁X₂X₃X₄YLQMNX₅X₆X₇X₈ as set forth in SEQ ID NO: 11, wherein X₁ and X₆ are each independently glycine (G) or proline (P), X₂ and X₅ are each independently lysine (K) or arginine (R), and X₃, X₄, X₅ and X₆ are each independently alanine (A) or nothing,
(b) YLQMN as set forth in SEQ ID NO:5,
(c) NMQLY as set forth in SEQ ID NO:16, or
(d) no more than 31, 30, 29, 28, 27, 26, 25, 24, 23, 22, 21, 20, 19, 18, 17, 16, 15, 14, or 13 amino acids in length, comprising or consisting of the amino acid sequence GKAANMQLYAAKG as set forth in SEQ ID NO:17,
said method comprising the steps of:
a) culturing the host cell of claim 18 under conditions and for a time sufficient to permit expression of the immunomodulatory polypeptide; and
b) isolating the immunomodulatory polypeptide from the cultured host cell.

20. An isolated polynucleotide that is selected from:
(a) an isolated antisense polynucleotide comprising a nucleic acid sequence that is complementary to the polynucleotide of claim 16,
(b) an isolated small interfering RNA (siRNA) polynucleotide that is capable of substantially silencing, and is complementary to a region of at least 18 and no more than 69 contiguous nucleotides in, a nucleic acid which encodes the immunomodulatory polypeptide of claim 1 or claim 2, and
(c) an isolated ribozyme that specifically binds to the polynucleotide of claim 16.

21. An isolated antibody, or antigen-binding fragment thereof, that specifically binds to the immunomodulatory polypeptide of claim 1 or claim 2.

22. The antibody of claim 21 which is a polyclonal antibody.

23. A pharmaceutical composition comprising the antibody of claim 21 and a physiologically acceptable carrier.

24. A method for detecting, in a biological sample, an immunomodulatory polypeptide that comprises the immunomodulatory polypeptide of claim 1 or claim 2, said method comprising the steps of:
(a) contacting the biological sample with an antibody that specifically binds said immunomodulatory polypeptide, or an antigen-binding fragment of said antibody, under conditions and for a time sufficient for specific antibody binding to the immunomodulatory polypeptide to take place; and
(b) detecting specific binding of the antibody to the immunomodulatory peptide, and thereby detecting the immunomodulatory peptide in the sample.

25. The method of claim 24 wherein at least one of:
(i) the antibody is a polyclonal antibody,
(ii) the antibody is linked to a support material,
(iii) the antibody is linked to a detectable label, or
(iv) the biological sample is obtained from a subject that is selected from a human, a non-human primate, a non-primate mammal, a non-mammalian vertebrate, an invertebrate eukaryote and a prokaryote.

26. A method of promoting implantation of an embryo in a pregnant or pseudopregnant mammal, comprising contacting at least one of the embryo and the pregnant or pseudopregnant mammal with an immunomodulatory polypeptide that is
(a) no more than 31, 30, 29, 28, 27, 26, 25, 24, 23, 22, 21, 20, 19, 18, 17, 16, 15, 14, 13, 12, 11, 10, or 9 amino acids in length, comprising or consisting of the amino acid sequence X₁X₂X₃X₄YLQMNX₅X₆X₇X₈ as set forth in SEQ ID NO: 11, wherein X₁ and X₈ are each independently glycine (G) or proline (P), X₂ and X₇ are each independently lysine (K) or arginine (R), and X₃, X₄, X₅ and X₆ are each independently alanine (A) or nothing,
(b) YLQMN as set forth in SEQ ID NO:5,
(c) NMQLY as set forth in SEQ ID NO:16, or
(d) no more than 31, 30, 29, 28, 27, 26, 25, 24, 23, 22, 21, 20, 19, 18, 17, 16, 15, 14, or 13 amino acids in length, comprising or consisting of the amino acid sequence GKAANMQLYAAKG as set forth in SEQ ID NO:17,
under conditions and for a time sufficient to promote embryonic implantation.

27. The method of claim 26, wherein the pregnant or pseudopregnant mammal is a human.

28. The method of claim 27, wherein the embryo is produced by in vitro fertilization.

29. A method for detecting, in a biological sample that comprises one or a plurality of nucleic acid molecules, expression of a polynucleotide that encodes an immunomodulatory polypeptide that is
(a) no more than 31, 30, 29, 28, 27, 26, 25, 24, 23, 22, 21, 20, 19, 18, 17, 16, 15, 14, 13, 12, 11, 10, or 9 amino acids in length, comprising or consisting of the amino acid sequence X₁X₂X₃X₄YLQMNX₅X₆X₇X₈ as set forth in SEQ ID NO: 11, wherein X₁ and X₈ are each independently glycine (G) or proline (P), X₂ and X₇ are each independently lysine (K) or arginine (R), and X₃, X₄, X₅ and X₆ are each independently alanine (A) or nothing,
(b) YLQMN as set forth in SEQ ID NO:5,
(c) NMQLY as set forth in SEQ ID NO:16, or
(d) no more than 31, 30, 29, 28, 27, 26, 25, 24, 23, 22, 21, 20, 19, 18, 17, 16, 15, 14, or 13 amino acids in length, comprising or consisting of the amino acid sequence GKAANMQLYAAKG as set forth in SEQ ID NO:17, said method comprising the steps of:
(a) contacting the sample with at least one of (i) the antisense polynucleotide of claim 20, and (ii) the polynucleotide of claim 16, under conditions and for a time sufficient for specific nucleic acid hybridization to occur; and
(b) detecting specific hybridization of at least one nucleic acid of the sample to at least one of said antisense polynucleotide of claim 20 and said polynucleotide of claim 16, and thereby detecting, in the sample, expression of the polynucleotide that encodes the immunomodulatory peptide.

30. The method of claim 29 wherein the biological sample is obtained from a subject that is selected from the group consisting of a human, a non-human primate, a non-primate mammal, a non-mammalian vertebrate, an invertebrate eukaryote and a prokaryote.

31. An isolated immunomodulatory polypeptide that competes with PeptideX2-13 or a variant thereof for specific binding to a human neutrophil, wherein said PeptideX2-13 comprises the amino acid sequence GKAAYLQMNAAKG as set forth in SEQ ID NO: 3, and wherein said variant thereof comprises or consists of
(a) the amino acid sequence of general formula X₁X₂X₃X₄YLQMNX₅X₆X₇X₈ as set forth in SEQ ID NO: 11, wherein X₁ and X₈ are each independently glycine (G) or proline (P), X₂ and X₇ are each independently lysine (K) or arginine (R), and X₃, X₄, X₅ and X₆ are each independently alanine (A) or nothing,
(b) YLQMN as set forth in SEQ ID NO:5,
(c) NMQLY as set forth in SEQ ID NO:16, or
(d) GKAANMQLYAAKG as set forth in SEQ ID NO:17.

32. The immunomodulatory polypeptide of claim 31 which interferes with PeptideX2-13 binding to either or both of human TLR2 and human TLR4.
